(19) United States
(12) Patent Application Publication
(10) Pub. No.: US 2009/0148486 A1
(43) Pub. Date: Jun. 11, 2009

(54) COMPOSITIONS AND METHODS FOR TREATING PULP INFLAMMATIONS CAUSED BY INFECTION OR TRAUMA

(76) Inventors: Helen Lu, New York, NY (US); Gunnar Hasselgren, New York, NY (US); Mona McAlarney, New York, NY (US)

Correspondence Address: WilmerHale/Columbia University, 399 PARK AVENUE, NEW YORK, NY, 10022, US

(21) Appl. No.: 11/927,581

(22) Filed: Oct. 29, 2007

Related U.S. Application Data
(63) Continuation-in-part of application No. PCT/US2006/015860, filed on Apr. 28, 2006.
(60) Provisional application No. 60/950,499, filed on Jul. 18, 2007.
(60) Provisional application No. 60/675,767, filed on Apr. 28, 2005.

(51) Int. Cl. A61K 9/10; A61K 35/12; C12Q 1/02

(52) U.S. Cl. 424/422; 424/93.7; 424/486; 435/29

(57) ABSTRACT

The present disclosed subject matter relates to methods and compositions for restoring a diseased or damaged tooth such that infection is inhibited or eliminated and pulp regeneration is facilitated. The disclosed subject matter also includes a composition comprising a physiologically acceptable matrix seeded with pulp cells. The matrix can be capable of being injected into the pulp chamber of a tooth. In some embodiments, the matrix of a composition includes a hydrogel (e.g., collagen, chitosan, alginate, MATRIGEL™, gelatin, JELL-O®, fibrin), a mesh (e.g., polylactide-coglycolide (PLGA) mesh, polylactide (PLA) mesh, or polyglycolide (PGA) mesh, a cross-linked fiber mesh, a nanofiber mesh, a mesh fabric, biodegradable polymer mesh), a microsphere (biodegradable polymer microsphere, a hydrogel microsphere), or a combination of any of the foregoing. In yet other embodiments, the matrix includes a nanofiber, an artificial three-dimensional scaffold material, or a synthetic three-dimensional scaffold material.

This application claims priority to provisional U.S. Application Ser. No. 60/675,767, filed on Apr. 28, 2005, provisional U.S. Application Ser. No. 60/950,499, filed on Jul. 18, 2007 and International Patent Application No. PCT/US2006/015860, filed Apr. 28, 2006, which are each herein incorporated by reference in their entirety.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

### BACKGROUND OF THE INVENTION

Inflammations of tooth pulp are commonly treated by procedures that remove the tooth pulp (e.g. endodontic, or root canal, treatment) or by extraction of the entire tooth. The first process results in devitalization of the tooth. This means that further dental hard tissue formation will not take place in young individuals. It is therefore desirable to develop an approach that promotes preservation of the tooth and the vitality of its pulp. Clinical considerations for such an approach are 1) the inability of present dental materials to provide permanent microorganism-proof seals, 2) a paradigm shift from the current clinical treatment modality of removing the affected pulp in young teeth to pulp regeneration, which enables continued dentinal self repair, extended growth of immature teeth, continued ability to fight infection, and the reduction of complications due to prosthetic repair of endodontically treated teeth, and 3) the lack of viable pulp regeneration grafts. Despite the ability of dental pulp to self-regenerate and repair under specific conditions in vivo, the current endodontic treatment modality for affected young pulp is total or partial removal of pulp tissue. Presently, pulp regeneration is generally not an aim of treatment. Rather, the goal is to inhibit and/or prevent infection by sealing off the pulp space.

Root canal therapy (RCT) is a complex clinical procedure that results in permanent loss of tooth vitality. At present, treatment forms for inflamed dental pulps either remove the pulp or, in young teeth, are aimed at healing at the location of the pulp wound or closer to the root. Moreover, there is no treatment form that is aimed at regaining pulp space lost to inflammation. Approximately 24 million root canal procedures are performed annually in the United States, with pulpectomies representing a major portion of these procedures. With a very conservative average cost of $400 per treatment this represents $9.6 billion per year for endodontic treatment alone. The tooth structure then must be restored with available dental materials. The restorative costs are approximately 14.4 billion dollars for an average restoration cost of $600. Therefore the total cost is approximately 24 billion dollars per year. Moreover, this cost is greatly underestimated since it does not include retreatment costs for failed therapies. It is clear that pulp-related endodontic diseases pose a significant clinical and social challenge.

Functional tissue engineering-based solutions focusing on the regeneration of excised pulp tissue are useful for harnessing the natural reparative capacity of pulp tissue and for enabling viable endodontic therapy with the potential to restore native tooth function. There have been reported attempts to obtain a biological seal in vivo after partial pulpotomy (Cvek, 1978, Zilberman et al, 1989; Mejare and Cvek, 1993) and the concept of pulp regeneration is being considered (for review see Murray et al, 2007, J. Endod. 2007 April; 33(4):377-90. Epub 2007 Feb. 20).

Studies have addressed the possibility of pulp regeneration and the following are some examples from the literature. Baboon pulpal chambers filled with an enriched collagen gel showed no histological difference between the collagen treated teeth and the intact untreated control teeth after 30 days (Fuks et al.). Similarly, using an enriched collagen solution as a capping agent in a canine model resulted in complete pulp regeneration within one month, while the controls remained inflamed. Results of the majority of studies using collagen or collagen-calcium phosphate based scaffolds showed variable responses, ranging from pulpal necrosis to a combination of mineralized and loose connective tissue. Pulpal cells grown in 3-D collagen gels can proliferate over time (Magloire et al., Mooney et al., and Brock et al.). The most recent studies utilizing collagen in or near the pulpal space as a carrier for both cells and cellular mediators reported no necrosis or inflammation. Therefore collagen can be well suited as a matrix if the gel-specific properties and clinical procedures are well controlled. A pulp-dentin like complex has been developed in vivo by utilizing cell transplantation (Young et al, 2005 et al., Tissue Eng. 2005 September-October; 11 9-10: 1599-610). As such, a scaffold system would be ideal for pulp tissue engineering, in order to control the healing process and provide a structural support for pulp regeneration. While pulp cells have been cultured on fiber-based scaffolds (Kaigler and Mooney, 2001; Rutherford, 2001), a gel-like matrix can be more optimal to mimic the ground substance of the pulp tissue, as well as enabling a better custom fit for the pulpal space. Currently, there is no optimal hydrogel-based scaffold for pulp tissue engineering, and methods for maintaining tooth viability and promote pulp repair/regeneration using the tissue engineered scaffold have not been fully explored. The current disclosed subject matter addresses these needs.

### SUMMARY OF THE INVENTION

The present disclosed subject matter relates to methods and compositions for restoring a diseased or damaged tooth such that infection is inhibited or eliminated and pulp regeneration is facilitated. In general, the disclosed subject matter encompasses compositions and methods that include 1) a matrix with pulp cells or stem cells that support new tissue formation in the matrix and pulp cell infiltration, 2) the matrix further containing at least one antibiotic incorporated into the matrix, 3) the matrix containing antibiotic, the antibiotic being incorporated into a delivery vehicle such as a degradable polymer-based microsphere, the vehicle being embedded in the matrix, 4) the matrix further containing at least one angiogenic factor that is incorporated into the matrix, 5) the matrix containing at least one angiogenic factor, the angiogenic factor being incorporated into a delivery vehicle such as a degradable polymer-based microsphere, and the vehicle being embedded in the matrix, 6) the matrix containing at least one angiogenic factor, the angiogenic factor being incorporated into a hydrogel, or an aligned degradable polymer-based nanofiber mesh (e.g., PLGA), the mesh being embedded in the matrix.

Accordingly, the disclosed subject matter also includes a composition comprising a physiologically acceptable matrix seeded with pulp cells. The matrix can include at least one agent that is an antibiotic or combination of antibiotics (e.g., ciprofloxacin, Minicyclin, and metronidazole), antifungal agent(s), or growth factor(s); platelet rich plasma (PRP) or any combination thereof. In some aspects of the disclosed subject matter, the agent is time released (i.e., an extended release composition). The matrix can be capable of being injected into the pulp chamber of a tooth. In some embodiments, the matrix of a composition includes a hydrogel (e.g., collagen, chitosan, alginate, MATRIGEL™, gelatin, JELL-O®, fibrin), a mesh e.g., polylactide-coglycolide (PLGA) mesh, polylactide (PLA) mesh, or polyglycolide (PGA) mesh, a cross-linked fiber mesh, a nanofiber mesh, a mesh fabric, biodegradable polymer mesh, a microsphere (biodegradable polymer microsphere, a hydrogel microsphere), or a combination of any of the foregoing. In yet other embodiments, the matrix includes a nanofiber, an artificial three-dimensional scaffold material, or a synthetic three-dimensional scaffold material. The matrix can include a polycaprolactone polymer, a poly-α-hydroxyester polymer, a polyanhydride polymer, or a combination of any of the foregoing. Certain aspects of the disclosed subject matter include a matrix that includes type I collagen and type III collagen, e.g., in a ratio of type I collagen to type III collagen is 30%:70%, 55%:45%, 45%:55%, or 70%:30%. In other aspects of the disclosed subject matter, the matrix includes type I collagen (e.g., the collagen concentration is about 0.3% to 3.0%, about 0.3% to 0.5%, or about 0.5% to about 3.0%). The gelation pH of the collagen matrix can be about 6.0, 7.5, or 9.0.

In one aspect, the disclosed subject matter provides for a composition comprising, (a) a matrix comprising (i) type I collagen, (ii) type III collagen, (iii) alginate, (iv) chitosan, (v) PEG-fibrinogen, (vi) type I collagen and type III collagen, (vii) type I collagen and alginate, (viii) type I collagen and chitosan, (ix) type I collagen and PEG-fibrinogen, (x) type III collagen and alginate, (xi) type III collagen and chitosan, (xii) type III collagen and PEG-fibrinogen, (xiii) alginate and chitosan, (xiv) alginate and PEG-fibrinogen, (xv) chitosan and PEG-fibrinogen, (xvi) type I collagen and type III collagen and alginate, (xvii) type I collagen and type III collagen and chitosan, (xviii) type I collagen and type III collagen and PEG-fibrinogen, (xix) type I collagen and alginate and chitosan, (xx) type I collagen and alginate and PEG-fibrinogen, (xxi) type I collagen and chitosan and PEG-fibrinogen, (xxii) type III collagen and alginate and chitosan, (xxiii) type III collagen and alginate and PEG-fibrinogen, (xxiv) type III collagen and chitosan and PEG-fibrinogen, alginate and chitosan and PEG-fibrinogen, (xxv) type III collagen and alginate and chitosan and PEG-fibrinogen, (xxvi) type I collagen and alginate and chitosan and PEG-fibrinogen, (xxvii) type I collagen and type III collagen and chitosan and PEG-fibrinogen, (xxviii) type I collagen and type III collagen and alginate and PEG-fibrinogen, (xxix) type I collagen and type III collagen and alginate and chitosan, (xxx) type I collagen and type III collagen and alginate and chitosan and PEG-fibrinogen or any combination thereof, and (b) pulp cells and/or stem cells.

In one embodiment, the matrix comprises type I collagen at a concentration of about 0.3% to about 3.0%, about 0.3% to about 0.5%, or about 0.5% to about 3.0%.

In another embodiment, the matrix comprises type I collagen at a concentration of about 1 mg/ml to about 4 mg/ml, about 2 mg/ml to about 3 mg/ml, about 1 mg/ml to about 2 mg/ml or about 3 mg/ml to about 4 mg/ml.

In yet another embodiment, the matrix comprises type I collagen and type III collagen, wherein the ratio of type I collagen to type III collagen comprises about 30%:70%, about 55%:45%, about 45%:55%, or about 70%:30%.

In still a further embodiment, the matrix comprises alginate at a concentration of about 1.0% to about 5.0%, about 1.0% to about 3.0%, or about 3.0% to about 5.0%.

In another embodiment, the matrix comprises chitosan at a concentration of about 1% w/v to about 5% w/v, about 1% w/v to about 2.5% v/w or about 2.5% w/v to about 5% w/v.

In one embodiment, the matrix comprises PEG-fibrinogen at a concentration of about 10% w/v. In still a further embodiment, the matrix comprises additional PEG-diacrylate, wherein the concentration of the additional PEG-diacrylate concentration comprises about 1% v/w to about 4% w/v, about 1% w/v to about 2.5% w/v or about 2.5% w/v to about 4% w/v.

In one embodiment, the viscosity of the matrix is less that 100,000 centipoise at 37° C.

In another embodiment, the gelation pH of the matrix is about 6.0, 7.5, or 9.0.

In yet another embodiment, the matrix comprises CaCl₂, at a concentration of about 50 mM to about 100 mM, about 100 to about 200 mM or about 50 mM to about 200 mM.

In still a further embodiment, the composition further comprises at least two antibiotics and at least one antibiotic is selected from the group consisting of ciprofloxacin, minicyclin, and metronidazole.

In one embodiment, the matrix further comprises platelet-rich plasma.

In another embodiment, the pulp cells are seeded at a density of about 0.5×10⁶ cells/ml, 1×10⁶ cells/ml, about 2×10⁶ cells/ml, about 3×10⁶ cells/ml or about 0.5×10⁶ cells/ml to about 3×10⁶ cells/ml.

In yet another embodiment, the pulp cells are selected from the group comprising: pulp-derived stem cells, progenitor cells, mesenchymal stem cells, pulp cells, bone marrow cells, embryonic stem cells, umbilical cord-derived, cells obtained from a cell culture or a combination thereof.

In one embodiment, the composition comprises at least one agent that is an antibiotic, antifungal agent, or growth factor.

In another embodiment, the composition comprises at least one time released agent that is an antibiotic, antifungal agent, or growth factor.

In one aspect, the disclosed subject matter provides for a composition comprising: (a) a scaffold of (i) an electrospun collagen mesh, (ii) an electrospun chitosan mesh, (iii) an electrospun alginate mesh, (iv) a poly(lactide-co-glycolide) mesh, (v) MATRIGEL™, (vi) gelatin, (viii) JELL-O®, (ix) a nanofiber, (x) extracellular matrix, (xi) a degradable polymer, (xii) a mesh of crosslinked fibers, (xiii) an artificial or synthetic three-dimensional scaffold material, (xiv) a polycaprolactone polymer, (xv) a poly-α-hydroxyester polymer, (xvi) a polyanhydride polymer, (xvii) a mesh fabric, (xviii) a polygalactin, (xxix) a polycyanoacrylate, (xxx) a polyphosphazene, or (xxxi) any combination thereof, and (b) at least one antibiotic or growth factor.

In another aspect, the disclosed subject matter provides a method for treating a subject having a pulp disorder or pulp damage within the pulp chamber of a tooth, the method comprising administering the composition of claim 1 to the subject.

In still a further aspect, the disclosed subject matter provides a method for treating a subject in need of a pulpectomy, a pulpotomy, a partial pulpotomy, pulp capping or root canal therapy, the method comprising administering the composition of claim 1 to the subject.

In one embodiment, the composition is administered to the pulp chamber in a tooth.

In another embodiment, the pulp chamber is substantially free of native pulp cells.

In a further embodiment, the pulp chamber comprises native pulp cells.

In yet another embodiment, the composition is inserted apically or coronally to the native pulp cells.

In one embodiment, the composition comprises a hydrogel, a mesh, a microsphere, or any combination thereof.

In another embodiment, the composition comprises microsphere selected from the group consisting of a biodegradable polymer microsphere, a hydrogel microsphere, or any combination thereof.

In still a further embodiment, the composition comprises a nanofiber, an artificial three-dimensional scaffold material, or a synthetic three-dimensional scaffold material or any combination thereof.

In yet another embodiment, the pulp cells can grow on the scaffold.

In another aspect, the disclosed subject matter provides for a kit comprising a physiologically acceptable matrix for seeding with pulpal cells and instructions for use, wherein the kit comprises a medium suitable for maintenance of harvested pulp cells, the kit comprises sealant suitable for sealing a tooth, or the kit comprises a chamber for culturing pulp cells on a matrix.

In another embodiment of the disclosed subject matter, the composition includes alginate, and the alginate concentration is e.g., about 1.0% to 5.0%, 1.0% to 3.0%, or 3.0% to 5.0%. A composition can include CaCl₂ (e.g., at a concentration of about 50 mM, about 100 mM, or about 200 mM).

In some embodiments, the matrix includes chitosan.

In certain compositions, the viscosity of the gel is less that 100,000 centipoise at 37° C. The matrix of certain compositions can, in some cases, forms a scaffold upon which the pulp cells can grow.

A composition can include one or more cell growth factors. Compositions can include cells, e.g., at least one of pulp-derived stem cells, progenitor cells, embryonic stem cells, umbilical cord cells, or mesenchymal stem cells. Such cells can be obtained from a subject or from a cell culture (e.g., cells that have migrated from a pulp explant or other tissue explant). In some embodiments, the cells are pulp cells, bone marrow cells, or a combination thereof.

In some compositions of the disclosed subject matter, the matrix degrades over time, e.g., after placement in a pulp chamber.

A composition can include chitosan.

In certain embodiments, the composition includes platelet-rich plasma (PRP) or platelet-rich plasma-derived growth factors (e.g., one or more growth factors that are in PRP, or PRP that is prepared to enrich for such growth factors). In some embodiments, the composition includes at least one of one or more bone morphogenic proteins (BMPs) or dentin powder.

In yet another embodiment of the disclosed subject matter, the matrix is seeded with about 1×10⁶ cells/ml, about 2×10⁶ cells/ml, or about 3×10⁶ cells/ml.

An aspect of the disclosed subject matter relates to a method that includes administering to a subject a physiologically acceptable matrix into the pulp chamber of a tooth. In some embodiments of the method, the physiologically acceptable matrix is seeded with cells. The composition used in the method can include a physiologically acceptable matrix capable of being injected into the pulp chamber of a tooth. In some embodiments, the pulp chamber is substantially free of native pulp cells. In other embodiments, the pulp chamber comprises native pulp cells. The composition can be, in some cases, inserted apically to the native pulp cells, or the composition can be inserted coronally to the native pulp cells. In certain embodiments, at least two different compositions are inserted into the pulp chamber. The composition can be administered using a method that includes injection (e.g., of a composition into the pulp chamber of a tooth).

In another aspect, the disclosed subject matter relates to a method for treating an individual having a pulp disorder or pulp damage within the pulp space of a tooth. The method includes administering a composition that includes a physiologically acceptable matrix into the pulp chamber. In some embodiments, the matrix is seeded with, e.g., pulp cells, embryonic stem cells, umbilical cord-derived cells, or mesenchymal stem cells. In certain embodiments of the method, pulp tissue is removed from the pulp chamber. In other embodiments, pulp tissue is not removed from the pulp chamber. The cells of a composition used in the method can be derived from the individual (e.g., the individual that is being treated). In other embodiments, the cells of the composition are not derived from the individual being treated. In certain embodiment, following injection of the composition, the pulp chamber is sealed.

The disclosed subject matter also relates to a composition comprising a scaffold of electrospun collagen, electrospun PLGA, degradable polymer, or chitosan mesh, wherein the scaffold comprises at least one antibiotic or growth factor. In some cases, the disclosed subject matter also relates to a method for culturing mesenchymal stem cells or pulp fibroblasts by culturing the cells on a scaffolding composition.

In another aspect, the disclosed subject matter relates to a method for culturing primary pulp cells. The method includes seeding the pulp cells that have migrated from a pulp explant in a matrix comprising hydrogel or other matrix as described herein. In certain embodiments, the cells are cultured in a hydrogel and the hydrogel is alginate or chitosan.

Also encompassed by the disclosed subject matter is a kit that includes a physiologically acceptable matrix for seeding with pulp cells and instructions for use. The kit can include, e.g., a medium suitable for maintenance of harvested pulp cells. In certain embodiments, the kit includes sealant suitable for sealing a tooth. In yet other embodiments, the kit includes a chamber for culturing pulp cell cells on a matrix.

Compositions as described herein are also useful in the preparation of a medicament, e.g., for treating a damaged or diseased tooth.

### BRIEF DESCRIPTION OF THE FIGURES

FIG. 1. Schematic of the tooth.

FIG. 2. Vital pulp therapy through pulp tissue engineering. FIG. 2A Schematic of a healthy molar tooth. FIG. 2B. Root Canal Therapy (RCT) through pulpectomy and root filling treatment. Pulp tissue has been removed, the canals are filled with a synthetic material (in black) and the crown is also restored with a synthetic (in black). FIG. 2C. Pulpotomy. The coronal pulp has been removed, and a capping agent (white) is placed atop the root pulp. Restorative material for the crown is shown in black. FIG. 2D. Pulp Tissue Engineering. The pulp tissue engineering scaffold is injected after pulpotomy to fill the access cavity. FIG. 2E. Induction of vascularization and interaction with the remaining pulp will lead to host-mediated coronal pulp regeneration following pulpotomy.

FIG. 3. Monomer structure of Chitosan

FIG. 4. FIG. 4A. Schematic of the PEG-fibrinogen hydrogel assembly. FIG. 4A PEG-fibrinogen hydrogel assembly is accomplished by photoinitiation of unreacted PEG-DA, resulting in a network of PEGylated fibrinogen FIG. 4B.

FIG. 5. Human pulpal cells obtained from explants growing on a culture flask surface. FIG. 5A Cells beginning to form an oriented structure. FIG. 5B Cells eventually obtain an oriented structure.

FIG. 6. Pulp cell cultures. FIG. 6A. Pulp cell monolayer culture at day 21. FIG. 6B. Pulp cells embedded in collagen gel at day 28. 10×, bar=100 μm.

FIG. 7. FIG. 7A. Pulp cell proliferation in type I collagen gel as a function of culturing time. FIG. 7B ALP activity for pulp cells grown in a type I collagen gel increased over time (reaction time=24 hours).

FIG. 8. Contraction of the collagen gel during in vitro culture with pulp cells. FIG. 8A. Day 1. FIG. 8B Day 28

FIG. 9A. A reproduction of a photomicrograph of pulp cells embedded in alginate beads on culture day 3. Original magnification was 10×. FIG. 9B. A reproduction of a photomicrograph of pulp cells embedded in a collagen type I gel on culture day 0. Original magnification was 10×.

FIG. 10. FIG. 10A. Fluorescent live staining images of pulp cells in chitosan hydrogel film, Day 3, 5×; FIG. 10B. Fluorescent live staining images of pulp cells in chitosan hydrogel film, Day 5, 5×.

FIG. 11. FIG. 11A. Pulp cells in chitosan hydrogel film Day 0, 10×. FIG. 11B. Pulp cells in chitosan hydrogel film Day 6, 10×

FIG. 12. FIG. 12A. Pulp cell morphology in PEG-fibrinogen gel over a three-week period, bar=100 μm. FIG. 12B Cell outgrowth (arrow) from the pulp explant into the PEG-fibrinogen hydrogel, Day 9, bar=100 μm.

FIG. 13. A bar graph depicting results of experiments assaying the effect of PRP on cell proliferation of pulp cells or endothelial cells (EC).

FIG. 14. Effects of PRP on CD31 (PECAM-1) deposition. FIG. 14 A Pulp Cells (Pulp). FIG. 14B Endothelial cells (EC), Day 14, bar=100 μm.

FIG. 15. A bar graph depicting results of experiments assaying cell proliferation in which pulp cells grown alone in monolayer were compared to pulp cells grown under co-culture conditions.

FIG. 16. A bar graph depicting the results of experiments assaying cell proliferation in which pulp cells grown in chitosan were compared to pulp cells grown in chitosan under co-culture conditions.

FIG. 17. A bar graph depicting the results of experiments assayed alkaline phosphatase (ALP) in a pulp cell monolayer compared to pulp cells grown under co-culture conditions. ALP activity is normalized and expressed as activity per cell.

FIG. 18. A bar graph depicting the results of experiments assayed ALP in pulp cells grown in chitosan (beads). ALP activity is normalized and expressed as activity per cell.

FIG. 19. A bar graph depicting the results of cell proliferation assays of human pulp cells cultured on aligned or unaligned nanofiber mesh.

FIG. 20. A bar graph depicting ALP activity of human pulp cells cultured on aligned or unaligned nanofiber mesh.

FIG. 21. SEM micrographs of cells 1 to 14 days after seeding. FIG. 21A is a reproduction of an SEM photomicrograph of an aligned nanofiber mesh (scaffold) one day after seeding (image ×500). FIG. 21B is a reproduction of an SEM photomicrograph of an unaligned scaffold one day after seeding (image ×500). FIG. 21C is a reproduction of an SEM photomicrograph of an aligned scaffold seven days after seeding (image ×500). FIG. 21D is a reproduction of an SEM photomicrograph of an unaligned scaffold seven days after seeding (image ×500). FIG. 21E is a reproduction of an SEM photomicrograph of an aligned scaffold 14 days after seeding (image ×500). FIG. 21F is a reproduction of an SEM photomicrograph of an unaligned scaffold 14 days after seeding (image ×500).

FIG. 22. A. Schematic of co-culture model B. Light micrograph of Co-culture of pulp cell+chitosan hydrogel with monolayer of pulp cells, day 4, 10×

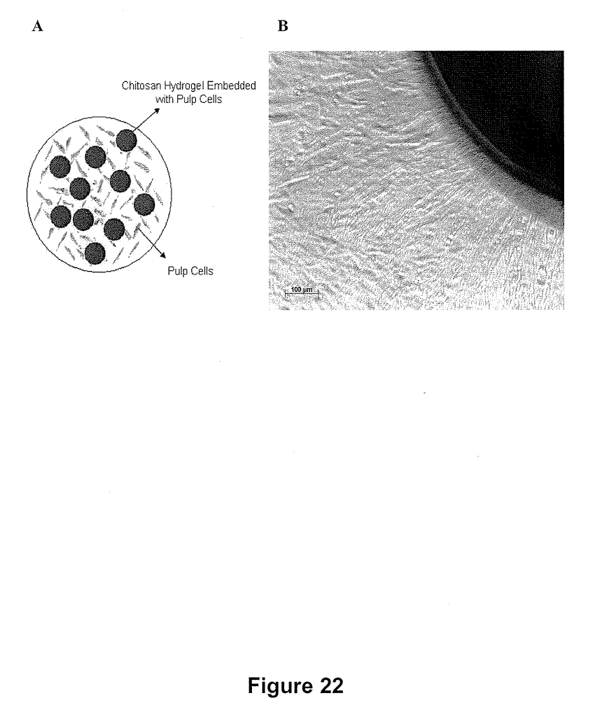

FIG. 23. Schematic of MSC cell culture.

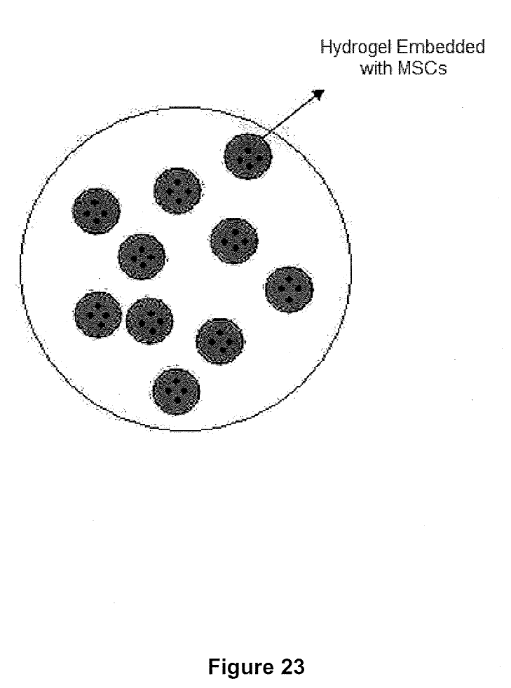

FIG. 24. Schematic of MSC+Pulp cell co-culture.

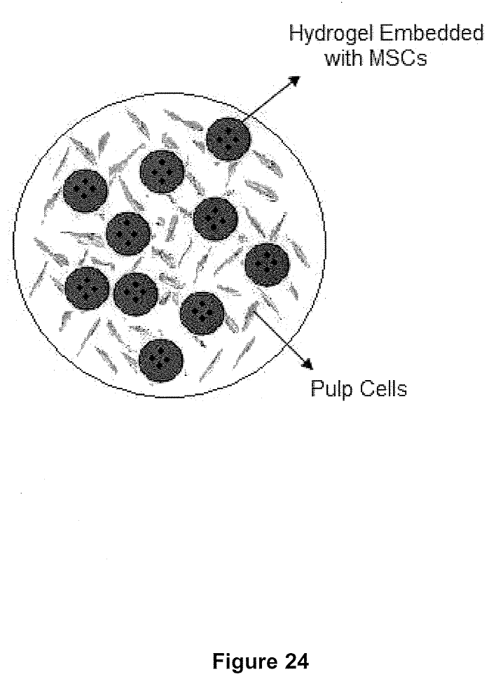

FIG. 25. A co-culture of endothelial cells with pulp cells in the absence and presence of VEGF showing the formation of blood vessels in the co-culture and induction of vascularization in tissue engineered pulp and promotion of healing and new pulp formation in vivo

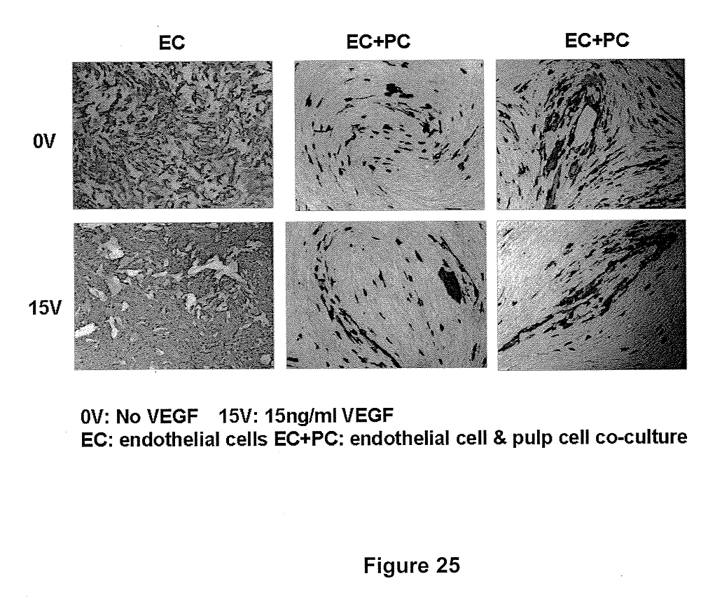

### DESCRIPTION OF THE INVENTION

The issued patents, applications, and other publications that are cited herein are hereby incorporated by reference to the same extent as if each was specifically and individually indicated to be incorporated by reference.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patent applications, published patent applications, issued and granted patents, texts, and literature references cited in this specification are hereby incorporated herein by reference in their entirety to more fully describe the state of the art to which the present disclosed subject matter pertains.

As various changes can be made in the methods and compositions described herein without departing from the scope and spirit of the disclosed subject matter as described, it is intended that all subject matter contained in this application and claims, shown in the accompanying drawings, or defined in the appended claims be interpreted as illustrative, and not in a limiting sense.

### DEFINITIONS

As used herein the language “physiologically acceptable” is intended to include any and all solvents, dispersion media, coatings, antibacterial and antifungal agents, isotonic and absorption delaying agents, and the like, compatible with physiological administration. The use of such media and agents for physiologically active substances is well known in the art. Except insofar as any conventional media or agent is incompatible with the active compound, use thereof in the compositions is contemplated. Supplementary active compounds can also be incorporated into the compositions.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the term “biomaterial” refers to a material that is designed and constructed to be placed in or onto the body or to contact fluid or tissue of the body. Ideally, a biomaterial will not induce undesirable reactions in the body such as blood clotting, tumor formation, allergic reaction, foreign body reaction (rejection) or inflammatory reaction; will have the physical properties such as strength, elasticity, permeability and flexibility required to function for the intended purpose; can be purified, fabricated and sterilized easily; and will substantially maintain its physical properties and function during the time that it remains implanted in or in contact with the body. Biomaterials can also include both degradable and nondegradable polymers.

As used herein, “scaffold” refers to an artificial, biocompatible malleable structure that can be used to deliver therapeutic compositions, e.g., proteins, peptides, nucleic acids, viruses, etc., into the body, to support and direct the growth of new cells of an organ or tissue. In addition, scaffold can be used to support cells that are implanted or “seeded,” and which can support three-dimensional cell growth, such as tissue or organ growth or regeneration. Scaffolds can be of natural or synthetic materials, and may be permanent, biodegradable, bioerodable or bioresorbable. Examples of natural scaffold materials include collagen, some linear aliphatic polyesters, chitosan, and glycosaminoglycans such as hyaluronic acid. Commonly used synthetic bioerodable scaffold materials include polylactic acid (PLA), polyglycolic acid (PGA); poly (lactide-co-glycolide) (PGLA) and polycaprolactone (PCL). In one embodiment, a scaffold can be a combination of mesh and hydrogel. Scaffolds generally have a high porosity to facilitate cell seeding and diffusion throughout the structure.

“Matrix” as used herein, refers to the surrounding substance within which something else originates, develops or is contained. The term matrix can also refer to a scaffold in which cells have been seeded. The matrix serving as a scaffold is typically a structure which can contain one or more structural components, for example collagen, and/or one or more other components such as cells, buffers, salts, extra cellular proteins, growth factors, etc. As used herein a matrix can be suitable for pulpal cell culture. The term matrix can also describe a biomaterial as well as the extracellular matrix produced by cells. In some embodiments of the invention, biomaterials or scaffolds are designed to imitate a naturally occurring extracellular matrix.

The term “mesh” means any material in any form including, for example, knotted, braided, extruded, stamped, knitted, woven, non-woven or otherwise, and may include a material with a substantially regular and/or irregular patterns. Examples of non-woven meshes include electrospun materials may be used (for a review on the preparation electrospun nanofiber material see Zheng-Ming Huang et al, Composites Science and Technology, 2003, 63:2223-2253). A mesh can be, without limitation, a cross-linked fiber mesh, a nanofiber mesh, a mesh fabric, biodegradable polymer mesh, or a combination of any of the foregoing. A mesh can be non-degradable, degradable or biodegradable. A degradable mesh can be a mesh that can be degraded via non-biological means (e.g. hydrolysis or photolysis). A biodegradable mesh can be a type of mesh that can be broken down by a biological system through the action of cells or digestive enzymes or via oxidation by biomolecules. Prior to implantation, the mesh may be trimmed or cut from a sheet of bulk material to match the configuration of the tooth space, or at a minimum, to overlay the exposed area. The mesh may be bent or shaped to match the particular configuration of the placement region.

As used herein, the term “hydrogel” refers to a polymeric material that exhibits the ability to swell in water and to retain a significant portion of water within its structure without dissolving. The hydrogel formed herein can chemically incorporate an active ingredient, such as a bioactive agent, that reacts with the components of the hydrogel-forming system; this can be accomplished by reacting the active ingredient with the components of the hydrogel-forming system herein. Active ingredients that are not reactive with components of the hydrogel-forming system herein can be physically entrapped within the hydrogel or physically encapsulated within the hydrogel by including them in the reaction mixture subjected thereby causing formation of hydrogel with the active ingredient entrapped therein or encapsulated thereby.

A used herein, the terms “pulpal cells” or “pulp cells” refer to cells that can be found in pulp tissue. Pulpal or pulp cells can be, without limitation, fibroblasts, odontoblasts, blood cells, perivascular cells, Schwann cells, endothelial cells, epithelial cells, pericytes, undifferentiated mesenchyma cells, polarized pulpal cells derived from the cranial neural crest, stem cells, and cells involved in the immune response, such as macrophages, mast cells, antigen processing cells (dendritic cells), B- and T-lymphocytes, neutrophils and plasma cells.

The disclosed subject matter provides a functional tissue engineering-based solution to regenerate dental pulp tissue in teeth that are damaged. The methods restore the tooth form of a damaged tooth and function to approximate those of the pre-affected tooth. The compositions comprise a matrix that has structural and functional properties that promote the growth and differentiation of pulp fibroblasts in vitro. Accordingly, the disclosed subject matter relates to methods of identifying compositions that are suitable for promoting the growth and differentiation of pulp fibroblasts in vitro.

The disclosed subject matter further provides a functional tissue engineering-based solution to regenerate dental pulp tissue in an affected tooth, e.g., in a tooth that is infected, was infected, or in which the tooth pulp was otherwise damaged, for example by physical trauma. The goal is to restore tooth form and function to approximate those of the pre-affected tooth, for example to preserve nerve innervation of the tooth.

Dental pulp tissue engineering for pulp repair and regeneration is attractive as it focuses on viable endodontic therapy with the potential to restore normal tooth function. In lieu of RCT, it is envisioned that the inflamed part of the pulp can first be removed via partial or full pulpotomy, or instrumentation into the root pulp, followed by stimulation of the remaining pulp tissue to regenerate and re-fill the original pulp cavity. Formation of this neo-pulp tissue will help to preserve tooth vitality and enable long term tooth function. In summary, we have proposed here an innovative approach to pulp tissue engineering which harnesses the natural repair potential of the dental pulp and offers significant promise for vital tooth therapy.

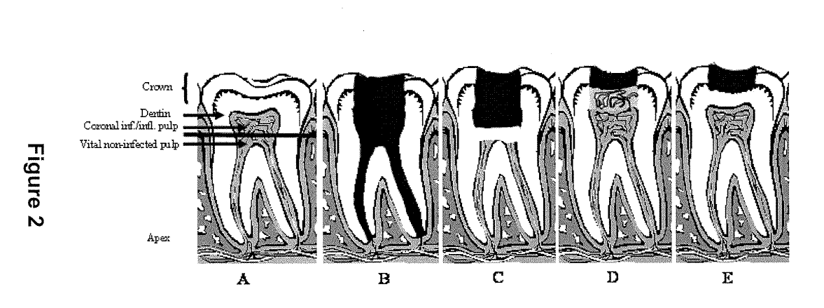

The disclosed subject matter provides compositions for use as a functional tissue engineering-based solution to regenerate dental pulp tissue. The disclosed subject matter provides methods to restore the tooth form and function to approximate those of the pre-affected tooth. The clinical significance of such an approach stems from 1) the need for pulp regeneration as reflected by the 24 million root canal therapies performed per year in the U.S., 2) a paradigm shift from the current clinical treatment modality of removing the affected pulp to pulp regeneration, which enables continued dentinal self repair, extended growth of immature teeth, continued ability to fight infection, and the reduction of complications due to prosthetic repair of endodontically treated teeth, 3) the inability of present dental materials to provide permanent microorganism-proof seals whereas pulp and dentin combined provides such a seal, and 4) the lack of viable pulp regeneration grafts. Despite the ability of dental pulp to self-regenerate/repair under specific conditions in vivo, the current major endodontic treatment modality for affected pulp is total or partial removal of affected pulp tissue. Depending on the clinical diagnosis, the treatment goal for teeth with affected pulp is to inhibit and/or prevent or treat infection, seal off the pulp space, enable continued root growth in pediatric patients and inhibit and/or prevent tooth discoloration. Pulp regeneration is not generally an aim of currently used treatments. The literature on pulp regeneration/repair is small compared to that of dentin regeneration/repair. The focus of treatment is generally on dentin since dentin provides the major form and function of the tooth. Yet, if affected pulp tissue can be regenerated clinically, the regenerated pulp alone can repair/regenerate dentin as it does naturally (FIG. 2).

The disclosed subject matter provides for a composition comprising a tissue engineered construct combining a biocompatible material either with or without a cellular component, which can be used as a solution for pulp regeneration/repair and hence provide for continued normal functioning of the tooth. A functional tissue engineering approach is utilized by the methods of this disclosed subject matter.

The disclosed subject matter encompasses a matrix capable of supporting pulp fibroblast growth and differentiation, while possessing structural and functional properties favorable for implantation.

The disclosed subject matter also provides a drug delivery system and a tooth explant organ culture. In some embodiments the disclosed subject matter also provides at least one of an antibiotic, antifungal, anti-inflammatory, or angiogenesis drug delivery systems that can be used in conjunction with the pulp regeneration construct.

A composition provided herein generally has specified structural and functional parameters such that the composition can fill the entire pulp space with a dimensionally stable material through which newly generated pulp can grow. This feature of a composition is desirable since voids in the pulp space can increase the chance of infection. The effects cells in the matrix of a composition can be significant since the construct/dentin interface integrity is important. A previous study showed that gel contraction for a collagen/GAG gel was greater in the presence of pulp-cells. ADA (American Dental Association) specification 57 is for endodontic sealants. In current endodontic practice the filled pulp space consists of points (typically thin cones of gutta percha) surrounded by sealant. The present disclosed subject matter relates to the use of a gel, which is not strictly analogous to endodontic sealants, the ADA 57 is used herein as a standard technique to measure gel properties. In another embodiment, the final tissue engineered construct can comprise the addition of point/cone-like material. This more rigid material can be used to deliver drugs or cells. In that case, the gel component of a composition is more analogous to endodontic sealants.

For pulp regeneration to persist, an infection-free and an angiogenic environment is important. An antibiotic delivery system, anti-fungal delivery system, and angiogenic compositions are encompassed by the disclosed subject matter.

The disclosed subject matter provides a functional tissue engineering-based solution to regenerate dental pulp tissue. Compositions and methods are provided to restore tooth form and function to approximate those of the pre-affected tooth. The compositions possess structural and functional properties favorable for implantation. In an embodiment, the disclosed subject matter provides a tissue engineered construct delivered to a damaged tooth combining a biocompatible material either with or without a cellular component that provides a solution for pulp regeneration/repair and consequently, restoring normal function of the tooth.

In general, the disclosed subject matter provides a composition comprising an injectable hydrogel matrix with appropriate viscosity, dimensional stability, microstructure, and mechanical properties for pulp-regeneration.

### The Pulpodentin Complex

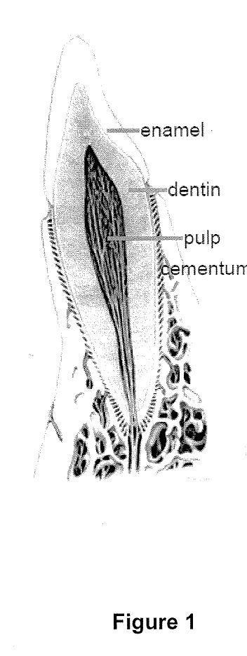

The human tooth is consisted of the pulp and three types of hard tissues (enamel, dentin, and cementum) (FIG. 1). The enamel is the outer hard layer in the coronal portion of the tooth that provides a cutting and grinding surface to prepare for the chemical digestion of food. Cementum covers the root surface and is the interface between the tooth and the fibers that connect the tooth to alveolar bone. The pulp is a loose connective tissue surrounded entirely by the dentin except at the tooth root apex, and it is the vital part of the tooth, containing cells, blood vessels, nerves, fibrous matrix, and ground substance. Both dentin and pulp are anatomically and functionally integrated in the pulpodentin complex. The pulp-dentin border is lined by odontoblasts, which are derived from pulp and are responsible for the production of dentin and mineralized tissue. The primary role of the pulp is to support the odontoblast layer. The nature of this support can be subdivided into four categories: formative, defensive, nutritive, and sensory. The major role of the odontoblast is dentin formation and repair/regeneration. When a tooth is injured, odontoblasts form more dentin and thereby maintain the vitality of the tooth. Although the pulp space decreases in volume after dentin regeneration/repair, this vital tooth remains capable of self-repair through dentin mineralization, growth (in immature teeth), fighting infection through host immune response, and sensation. If dentin repair/regeneration is not successful the traumatized pulp becomes necrotic due to invading bacteria. The greatest clinical implication of non-vital pulp is a high infection rate resulting in periradicular disease, bone loss and the cessation of growth for immature teeth.

The dental pulp is the vital component of the tooth; its inherent vascularity and resident stem cell population enables the pulp to fight infection, support odontoblast-mediated dentin formation and repair, as well as provide nutrition and innervation to the tooth. In addition to supplying nutrition, the pulp provides new odontoblasts for dentin repair in the case of infection or traumatic injury. The pulp matrix is a non-mineralized connective tissue rich in types I and III collagen, contains non-collagenous proteins such as fibronectin, osteonectin, osteopontin and dental sialoprotein. The proteoglycan components of the pulp extracellular matrix include decorin, biglycan, and versican (Goldberg and Smith, 2004). The dental pulp also has an extensive vascular supply and the circulation is dynamic within the pulp space (Kishi and Takahashi, 1995). Anatomically, blood vessels and nerves enter the pulp through the apical foramen (Mjör and Heyeraas, 1998) and penetrate through the main part of the pulp to the coronal portion of the pulp space (Suda and Ikeda, 2002). These main vessels branch out into terminal arterioles, pre-capillaries and capillaries to supply the pulp space with nutrients and facilitate waste removal. It has been reported that over 15% of the pulp is occupied by blood vessels, with a higher density in the central portion (42.9%) (Vongsavan and Matthews, 1992). The pulp vasculature is considered a microcirculatory system, with the largest vessels being arterioles and veins (Zhang et al, 1998). Following injury, angiogenic factors such as Transforming Growth Factor-beta (TGF-β), Platelet Derived Growth Factor (PDGF), Fibroblast Growth Factor-2 (FGF2), and Vascular Endothelial Growth Factor (VEGF) are released from the pulp (Derringer and Linden, 1998). Expression of VEGF has been detected only in the inflamed pulp (Guven et al, 2007). The vascular supply in dental pulp is therefore essential for pulp function, defense against infection and respond to injury. The cells derived from pulp represent a heterogeneous population of cells, with varying metabolic and proliferative activities. While the fibroblast is the dominant cell type, odontoblasts, endothelial cells, and mesenchymal stem cells are also found within the pulp tissue (d'Aquino et al, 2007). Pulp cells can be stimulated to adopt an odontoblast-like morphology and form mineralized nodules in vitro (Couble et al, 2000). Gronthos et al. isolated a clonogenic and mitogenic population of cells from adult pulp, and demonstrated the ability of these cells to form dentin-like tissue after transplantation into immuno-deficient mice (Gronthos et al, 2002). The dentin-like structure was lined with odontoblast-like cells and a pulp-like structure was also formed. It has been postulated that pulp cells play a significant role in angiogenesis. Pulp cells are known to secret angiogenic factors such as FGF2 and VEGF which can promote vascularization (Tran-Hung et al, 2006). Moreover, injured endothelial cells recruit pulp cells that organize and align along the injury site (Mathieu et al, 2005). Stems cells residing in dental pulp co-differentiate into osteoblasts and endothelial cells, with 30% of the cells expressing angiogenic markers such as CD45, CD31 or platelet-endothelium cell adhesion molecule (PECAM-1), and von-Willebrand factor (d'Aquino et al, 2007). It is likely that these pluripotent stem cells in the pulp tissue can be exploited for angiogenesis, pulp repair and regeneration.

### Current Treatment Options—Root Canal Therapy

While the dental pulp is encased by dentin and enamel, it can be exposed to the oral cavity through caries or trauma and rendered susceptible to infection. The clinical standard for the treatment of pulp inflammation is root canal therapy (RCT) (Hargreaves and Goodis 2002), during which pulpectomy or the complete removal of the pulp tissue (extirpation), is performed (FIG. 2). The pulp space is subsequently cleaned, shaped, disinfected, and then filled with an inert synthetic material. The tooth is often restored with a core build-up and a crown to replace the tooth structure.

### Vital Pulp Therapies

Although widely practiced, pulpectomy (removal of pulp tissue) is not the only treatment option. If pulp damage is considered reversible, a form of vital pulp therapy can be utilized, where only minimal pulp tissue is removed (pulpotomy or partial pulpotomy) or a wound dressing is placed directly on the exposed pulp (pulp capping). The coronal portion of the pulp space is then sealed and mineralization stimulated. The choice of clinical treatment depends on the nature of the pulp injury, age of the patient, and the presence or possibility of infection. The use of vital pulp therapies is particularly attractive for pediatric patients where the lack of continued tooth growth in pulpectomized teeth is problematic. Vital pulp therapies (pulp capping, partial pulpotomy, and pulpotomy) leave live pulp tissue in the tooth (FIG. 2). Still, the pulp capping procedure has an increased failure rate with increasing time and dentin barriers formed after pulp capping are often incomplete and leakage under a filling will bring bacteria in direct contact with the pulp tissue (Nyborg, 1955; Cox et al, 1996, Hörsted et al 1985, Barthel et al 2000). The other two methods have a better outcome, but the pulp space lost to infection/inflammation is not regained with these procedures. A further limitation of vital pulp therapies is that mineralization occurs only apically to the level of the vital pulp. Regeneration of dentin or dentin like tissue coronal to the pulp wound does not occur and the dental anatomy must be restored with dental materials. No current dental material can adequately duplicate the natural tooth with respect to mechanical properties and microleakage seal.

Tooth fractures in immature teeth treated with pulpectomies can occur at the bone level, thereby complicating or inhibiting and/or preventing restoration. Partial pulpotomy treatment, which is carried out at a level 1-2 mm under a pulp exposure, preserves the cervical area of the pulp thus making continued dentin formation possible. This increases the hard tissue bulk also in the cervical area inhibiting and/or preventing fractures. In carefully controlled studies, the success rates are high (91-93%) for partial pulpotomy, however its application is also limited to young, posterior, and otherwise symptom-free teeth with carious exposures. Emphasis is on physically sealing the pulp space through a mineralized barrier formed by the pulp and the use of dental materials to inhibit and/or prevent further damage. Although the pulpal space decreases in volume after dentin repair following pulp capping, partial pulpotomy or pulpotomy, the vital tooth remains capable of self repair through dentin formation, root growth (in immature teeth), and defense against infection through host immune response. These procedures while promising, are however limited to younger patients with pulps that are exposed, but not severely damaged or infected. A limitation of present vital pulp therapies is that mineralization occurs only apically (towards the tip of the root) to the level of the vital pulp exposure. Regeneration of dentin or dentin like tissue coronal to the pulp wound does not occur and the dental anatomy must be restored with dental materials. No current dental material can adequately duplicate the natural tooth with respect to mechanical properties and micro-leakage seal. The restorations also tend to be large which accentuates the limitations of dental materials.

### Clinical Significance of Pulp Injuries

Depending on several factors, failure rates of 3-60% are reported for endodontic procedures. Most endodontic procedures are initiated because of pulp inflammation caused by infection stemming from caries. The treatment in the majority of cases involves pulpectomy. Pulpectomy is the removal of all pulp tissue, where the pulp space is cleaned, shaped, disinfected, and filled with a synthetic material. The tooth is then restored, often with a core build-up and a crown to replace tooth structures and function. The tooth is non-vital yet the structure of the tooth remains and the negative effects of tooth extraction are avoided. Nevertheless the treatment is both complex and time consuming. The technical complexity is reflected in the difference in failure rates between general dentists (30-35%) and endodontic specialists (5-15%) (Friedman, In: Orstavik and Pitt Ford, (eds.): Essential Endodontology Prevention and Treatment of Apical Periodontitis, Oxford, Blackwell Science, 1998). If a low 20% failure rate is assumed, the re-treatment cost is at least 4.8 billion dollars. The total re-treatment cost is higher since the failure rate of retreated teeth is significantly higher than the first treatment. Also increased damage to the tooth and supporting structures often occurs, thereby complicating treatment and increasing costs. Non-quantifiable costs such as time spent and pain are not included.

Clearly an important limitation of current treatment is the high failure rate. One of the causes of failure is the complexity and sensitivity of the treatment technique. Another cause is the nature of the treatment. When all pulp tissue is removed the tooth is susceptible to infection for several reasons. One reason is that current dental materials cannot adequately seal the apical and/or coronal ends of the pulp space. Dentin cannot regenerate/repair without pulp support. Bacteria entering the pulp space have a rich nutritive source as they demineralize dentin. And these bacteria are almost impossible to treat since the pulp immune response no longer exists and systemic antibiotics cannot reach the pulp space.

### Pulp Tissue Engineering

In one aspect of the disclosed subject matter, tissue engineering can be used as a methodology for ex vivo tissue regeneration for vital pulp therapy. Dental pulp tissue engineering for pulp repair and regeneration focuses on viable endodontic therapy with the potential to restore normal tooth function. In lieu of RCT, the inflamed part of the pulp can first be removed via pulpotomy, and the remaining pulp tissue can be stimulated to regenerate and re-fill the original pulp cavity (FIGS. 2D-2E). Formation of this neo-pulp tissue can help to preserve tooth vitality and enable long term tooth function. This proactive approach to dental pulp repair is innovative as it harnesses the natural reparative potential of the pulp tissue. For pulp repair, the matrix can be applied after removal of the infected part of the pulp following partial pulpotomy. Cell infiltration from the remaining pulp into the matrix can facilitate new pulp formation and host-mediated repair. For pulp regeneration, the entire pulp space can be filled with the pulp tissue engineering matrix, and matrix-mediated infiltration of host stem cells and blood vessels from the apex of the tooth into can promote pulp formation and eventual integration of the graft with host tissues. The matrix system can be acellular if it is laden with bioactive molecules that can promote pulp repair and regeneration, or it can be seeded with stem cells or progenitors which can expedite the repair or regeneration process. As shown in FIG. 2D, the tissue engineered scaffold can be placed occlusal to the pre-trauma pulpo-dentin interface, it is thus possible to restore both the dentin and the pulp to an anatomically more correct level than with current techniques. This approach can be suitable for patients who are candidates for vital pulp therapy as well as patients with vital teeth who now must be treated with pulpectomy due to advanced stages of pulp inflammation. With this method, not only can maximal tooth structure be regenerated, but the required traditional restoration would be minimal. This approach represents a paradigm shift in current treatment of the exposed and inflamed pulp, and through pulp tissue engineering, the ability for pulpal self-repair and sensation can be re-established.

### Matrices and Matrix Compositions

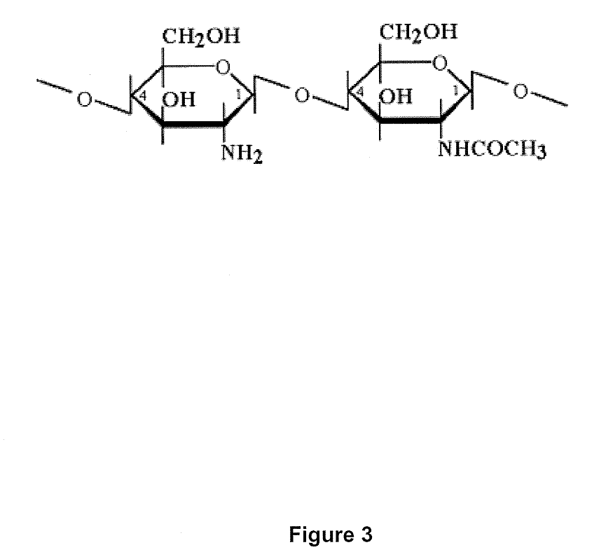

Several types of scaffolding matrices can be adopted for use with the compositions and methods of the disclosed subject matter. Chitosan is a polysaccharide biopolymer derived from chitin, found primarily in the exoskeleton of crustaceans. Next to cellulose, chitin is the second most abundant polymer found in nature (Chio et al, 2002). Chitosan is formed by deacetylating chitin, and its structure consists of a co-polymer of N-acetyl-glucosamine and N-glucosamine (FIG. 3). Chitosan has been used experimentally for wound healing (Ma et al, 2003; Sarasam et al, 2005; Mori et al, 1997), as well as bone (Park et al, 2003; Muzzarelli et al, 1993; Cho et al, 2004) and cartilage tissue engineering (Zielinski and Aebischer 1994; Park et al, 2005; Suh et al, 2000). It is an attractive substrate for pulp tissue engineering as chitosan has been shown to augment the immune response against bacteria, viruses and cancerous cells (Nishimura et al, 1987; Klokkevold et al, 1999). It has also been applied commercially as fruit coating to prevent bacterial growth (Rabea et al, 2003). Although the precise mechanism behind the antibacterial potential of chitosan is not fully understood, it is proposed that an inhibition of bacterial mRNA synthesis is achieved via the interaction of chitosan with DNA. Chitosan is degraded by enzymatic hydrolysis through the actions of lysozomes (Etienne et al, 2005). The degradation rate is inversely related to the degree of deacetylation. Chitosan has also been used in dental applications. Of 63 cases of periodontitis treated with chitosan gels, 52 cases found a significant reduction in tooth mobility and pocket depth as well as an enhancement in the regeneration of architectural organization (Muzzarelli et al, 1989). When chitosan powder was applied in periodontal pockets, palatal wounds and extraction sockets, significant wound healing was observed with the matrix group (Sapelli et al, 1986). These reports suggest that chitosan is a promising material for dental tissue engineering.

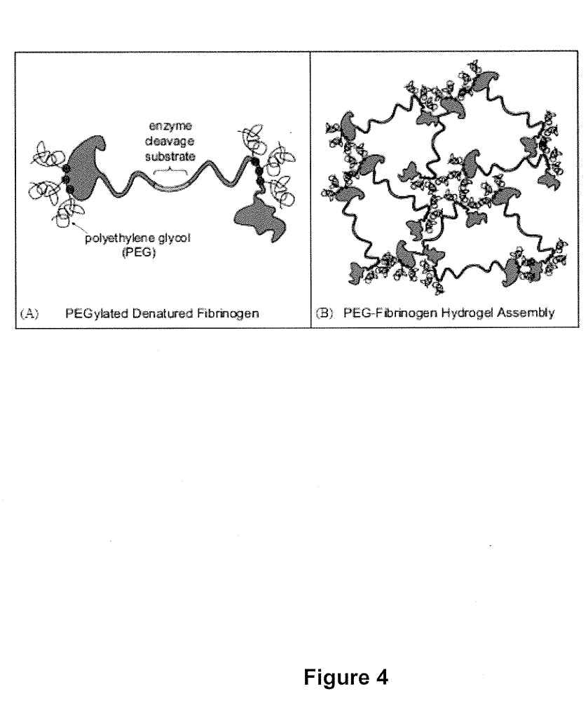

Another type of hydrogel is formed by combining polyethylene glycol (PEG) and fibrinogen fragments into a composite polymeric hydrogel (Almany and Seliktar, 2005) (FIG. 4). The PEG hydrogel has documented biocompatibility and its physical characteristics can be controlled by varying polymer weight percent, molecular chain length, and crosslinking density (Temenoff et al, 2002; Dikovsky et al, 2006). An additional advantage of PEG hydrogels is their ability to undergo a controlled liquid-to-solid transition (gelation) in the presence of a cell suspension (Elbert and Hubbell, 2001). The PEG gelation reaction can be carried out under nontoxic conditions in the presence of a photoinitiator (Elisseeff et al, 2000; Nguyen and West, 2002) or by mixing a two-part reactive solution of functionalized PEG and crosslinking the constituents (Lutolf and Hubbell, 2003). The Fibrinogen backbone of the PEG-fibrinogen gel serves as a natural substrate for tissue remodeling (Herrick et al, 1999), and provides the PEG-fibrinogen hydrogels an inherent degradability by way of cell-activated protease activity and cell specific adhesivity that are not available with PEG alone (Elbert and Hubbell, 2001). This promising hydrogel has been tested for both cartilage and cardiac muscle tissue engineering (Almany and Seliktar, 2005; Dikovsky et al, 2006; Seliktar, 2005) but its potential for pulp tissue engineering has not yet been assessed.

In one aspect of the disclosed subject matter, collagen mixtures are used in a composition that can be used for pulp regeneration and repair. Native pulp tissue is comprised of approximately 55% type I and 45% type III collagen. One biomimetic composition includes about 55% type I collagen and about 45% type III collagen. Compositions can be optimized based on cellular response and the need to match mechanical properties of the native tissue.

Structural properties that can be evaluated as a function of matrix properties include overall surface and matrix morphology, porosity, and fiber distribution.

Examples of collagen matrices include a type I collagen matrix having a type I collagen concentration of, e.g., about 0.2%-5%, about 0.3%-3.0%, 0.3%, 1.5%, or 3.0% (w/v). Gelation is carried out at a selected pH, e.g., pH 5.0-9.5, 6.0-9.0, 6.0, 7.5, or 9.0.

In some aspects, the matrix is a composite of type I collagen and type III collagen. Non-limiting examples of compositional ratios of type I to type III collagen include 30%:70%, 55%:45%, and 70%:30% (w/v) and gelation at a selected pH, e.g., pH 5.0-9.5, 6.0-9.0, 6.0, 7.5, or 9.0.

In other aspect, the matrix includes chitosan. Chitosan forms a gel in solutions with a pH above 12, and the gelation occurs at pH of about 9 in 10% amino acid solutions.

The matrix can also be an alginate matrix having an alginate concentration of, e.g., 0.5%-6.0%, 1.0%-5.0%, 1.0%, 3.0%, or 5.0 (w/v). The degree of gelation of an alginate matrix is generally regulated by selecting the CaCl₂ concentration. Non-limiting examples of CaCl₂ concentrations include concentration from about 25 mM-300 mM, 50 mM-200 mM, 50 mM, 100 mM, and 200 mM. In general, the variations disclosed herein in gel matrix material and gelation conditions are tested in methods for identifying compositions and constructs suitable for use in vital tooth repair, e.g., for administration to the pulp space of a damaged tooth.

The matrix can be a chitosan matrix. A chitosan matrix can have a concentration of, e.g., about 1% to about 5%, about 1.5% to about 3%, about 2% to about 4%, about 1% to about 3%, or about 2% to about 3%, e.g., about 2.5%. Methods of preparing a chitosan gel are known in the art, e.g., using glutaraldehyde. Selection of an optimal chitosan matrix can include selecting the degree of deacetylation of the chitosan (e.g., at least about 70%, at least about 80%, at least about 90%, or at least about 95%).

The matrix can also be a PEG-fibrinogen. A PEG-fibrinogen matrix can have an initial concentration of about 10% w/v. Additional PEG-diacrylate can be added to the precursor solution to improve the integrity of the construct. Additional amounts of PEG-diacryate can be, e.g., about 1% to about 4%, about 1.5% to about 3%, about 2% to about 4% or about 1% to about 3% additional PEG content. Methods of preparing PEG-fibrinogen gel are known in the art.

Useful matrices for culturing cells as described herein can also include an electrospun mesh, e.g., made using collagen, chitosan, or alginate. Alternatively, the mesh can be composed of other polymers. Polymer meshes are generally composed of a biodegradable material such as poly(lactide-co-glycolide) (PLGA). Methods of making electrospun mesh are known in the art. In general, the mesh is an aligned mesh, however, the mesh can be only partially aligned or can be unaligned. The mesh can include additional components such as antibiotics and growth factors (Katti et al, 2004)

Other matrix materials that can be used include hydrogels, MATRIGEL™, gelatin, JELL-O®, a nanofiber, extracellular matrix, a degradable polymer, a mesh of crosslinked fibers, an artificial or synthetic three-dimensional scaffold material, a polycaprolactone polymer, a poly-α-hydroxyester polymer, a polyanhydride polymer, a polygalactin, a polycyanoacrylate, a polyphosphazene, a mesh fabric, or a combination of any of the foregoing. Methods of preparing such materials are known in the art.

### Methods of Increasing Vascularization

Vascularization can be induced by a pulp construct containing a composition disclosed herein by, for example, the incorporation of growth factors such as platelet-derived growth factor (PDGF) and vascular endothelial growth factor (VEGF) into polymer microspheres that are included in a composition with a matrix such as a collagen gel, chitosan, PEG-fibrinogen or alginate. Microspheres containing factors are generally embedded in the gel of the matrix. Other growth factors known in the art such as EGF (epidermal growth factor) and derivatives thereof or a bone morphogenetic factor (BMP) can be used. Platelet-rich plasma (PRP) and other biological preparations having properties such as promotion of cell proliferation or cell differentiation (e.g., of pulp cells) can be used in a composition. PRP is derived from an autologous concentration of platelets.

Platelets are known to contain several growth factors including Transforming Growth Factor-beta (TGF-β), Platelet Derived Growth Factor (PDGF), Insulin-like Growth Factor (IGF), and Vascular Endothelial Growth Factor (VEGF) which can be released upon activation of the gel.

Platelet-rich plasma (PRP) is derived from blood plasma and serves as an autologous source of growth factors important in vascularization and bone regeneration. In the early stages of wound healing, platelets are activated by thrombin and collagen, and growth factors are released by these activated platelets to facilitate repair and healing. The sequence of events leading to bone formation (chemotaxis, cell migration, proliferation, and differentiation) are regulated by growth factors, many of which are present in PRP. For example, the recruitment of mesenchymal stem cells and progenitor cells to the site of bone regeneration is mediated by collagen as well as chemotactic factors such as PDGF and TGF-beta1. Moreover, PDGF and TGF-beta1 stimulate cell proliferation, and TGF-beta1 also induces the osteogenic differentiation of mesenchymal stem cells. Osteoblasts differentiation is controlled by IGF-I, IGF-II and bone morphogenetic proteins, and VEGF is critical in stimulating the angiogenesis necessary for bone formation and remodeling.

Various preparation methods have disclosed concentrations 7-120 ng/ml for PDGF-AB and 200 ng/ml was reported for VEGF in PRP. Angiogenic growth factors in PRP, such VEGF and PDGF, have been reported to be upregulated as part of the pulp repair response to trauma (Derringer and Linden, 2004; Derringer and Linden, 2003; Derringer and Linden, 1998). In addition, PRP induces healing in other soft tissues (Tran-Hung et al, 2006; Anitua et al, 2005; Murray et al, 2006; Murray et al, 2007).

A second means of encouraging vascularization in a matrix used in a composition is to deposit the above relevant growth factors directly onto a nanofiber mesh using an electrospinning process. This growth factor-containing mesh is embedded inside a hydrogel. This nanofiber mesh serves at least three purposes 1) as the reservoir for angiogenic growth factors, 2) the alignment of the mesh (in the case of an aligned mesh) can guide the formation of blood vessels, and 3) the nanofiber mesh can degrade and make room for vascular ingrowth, cell proliferation, and deposition of extracellular matrix by cells.

Similar methods are used for induction of vascularization in a tooth implanted with a construct described herein. In this case, agents that induce vascularization are included in the composition, e.g., in microspheres or using some other delivery method known in the art. Non-limiting examples of vascularizing agents include VEGF or basic fibroblast growth factor (bFGF).

### Identification of Suitable Matrix Compositions

Functional properties of a composition that are assayed can include dimensional stability in wet and dry conditions, gel viscosity, as well as elastic modulus. In general, a matrix includes a gel (e.g., a hydrogel such as collagen, alginate, chitosan, MATRIGEL™, gelatin, JELL-O®, polyethylene glycol (PEG), modified PEG, or fibrin), a mesh, a microsphere, and a combination of any of the foregoing. Additional compounds that can be used in a matrix include, without limitation, polylactide-coglycolide (PLGA) mesh, polylactide (PLA) mesh, or polyglycolide (PGA). A mesh can be, without limitation, a cross-linked fiber mesh, a nanofiber mesh, a mesh fabric, biodegradable polymer mesh, or a combination of any of the foregoing. Microspheres can be made of any suitable substance, e.g., a biodegradable polymer, a hydrogel, or a combination of any of the foregoing. Also useful for a composition that is a matrix that is a nanofiber, an artificial three-dimensional scaffold material, or a synthetic three-dimensional scaffold material. Additional compounds that can be used as a matrix in the disclosed subject matter include, without limitation, a polycaprolactone polymer, a poly-α-hydroxyester polymer, a polygalactin, a polycyanoacrylate, a polyphosphazene, or a polyanhydride polymer.

A matrix useful in the disclosed subject matter conforms to the following matrix selection criteria (termed “optimum criteria” herein). Optimum criteria for a matrix useful for tooth pulp repair can include one or more of the following; 1) low viscosity (<100,000 centipoise), 2) a setting contraction of less than 10%, 3) a post-setting contraction of less than 1% or less than 0.1% expansion of the matrix after 30 days, and 4) maintenance of structural integrity for up to 30 days, 5) a porous structure that will support cell growth and infiltration as well as nutrient transport, and 6) limited mineralization.

The disclosed subject matter also provides methods for examining the in vitro response (proliferation, differentiation, 3-dimensional cell structure formation, vascularization and angiogenesis) of pulp fibroblasts to the biodegradable gel matrix (e.g. a matrix containing collagen, chitosan, PEG-fibrinogen or alginate), as well as the effect of cell culturing on gel matrix properties (gel contraction and matrix organization). The ability of a matrix to support the growth and differentiation of pulp fibroblasts can be tested as described herein to determine suitability of a matrix for use in tooth repair. There can be a balance between mechanical properties and optimal cellular response (e.g., a highly porous surface can be favorable for cell attachment, but may not have the dimensional stability desired for long term functionality). Thus, certain criteria used for evaluating a matrix are cellular response to an optimized matrix, e.g., cell proliferation and cell differentiation.

A composite matrix of type I and III collagens is one useful matrix as it mimics the native composition of the human pulp, and it can support cell proliferation and differentiation. The presence of type III collagen in a matrix can reduce undesirable mineralization compared to a matrix composed of type I collagen alone.

In some applications, hydrogels such as alginate, PEG-fibrinogen and chitosan have advantages compared to collagen. For example, hydrogels can be more economical, they can be crosslinked using agents that are not cytotoxic, and, in the case of chitosan, the hydrogel has antibacterial properties that are useful for inhibiting and/or preventing or ameliorating infection in a tooth when a construct is used for treatment of a damaged tooth.

Chitosan is useful for compositions as described herein. Chitosan is a degradable biopolymer derived from the exoskeleton of crustaceans. The biocompatibility of chitosan is well documented as its anti-bacterial potential. Bacterial infection compromises the pulp vitality and is the primary clinical reason for performing RCT. Accordingly, a use of a matrix such as chitosan that has anti-bacterial properties is useful as a matrix for culturing pulp cells and for compositions for tooth repair.

Pulp fibroblast growth (DNA content) and differentiation (alkaline phosphatase, types I and III collagen production, mineralization, and the expression of osteocalcin and dental sialophosphoprotein) can be examined as a function of matrix type (type I collagen, type I and III collagens, chitosan, or alginate) as well as culture duration (e.g., 1 day, 3 days, 7 days, 14 days, 21 days, or 28 days).

The effect of cell seeding density (e.g., about 1×10⁶, about 2×10⁶, or about 3×10⁶ cells/ml) on gel matrix properties (gel contraction and matrix organization) are determined over time (e.g., 1 day, 3 days, 7 days, 14 days, 21 days, or 28 days) to identify conditions that are optimum for compositions containing cells.

Proliferation and differentiation are parameters that can be assessed in both restrained and non-restrained gels to identify an optimum matrix. The optimal gel matrix for pulp tissue engineering is defined as the system that can support pulp fibroblast proliferation and differentiation without causing excessive gel contraction or ectopic mineralization.

The rationale behind the design criteria is several fold. First, the entire empty part of the pulp space can be filled with the tissue engineered construct (matrix plus other components) to inhibit and/or prevent infection and permit optimal control of regeneration. To this end, the gel can have a sufficiently low viscosity, so it is injectable and can be used to fill the pulp space. If a gel (e.g., hydrogel) is to be placed in a root canal, a low gel viscosity is beneficial. For example, appropriate viscosities are the same or similar to the viscosities of dental materials such as zinc phosphate and zinc polycarboxylate cements, as well as light consistency silicone and polysulfide impression materials (<100,000 centipoise 2 minutes after mixing) (Vermilyea et al, 1977; Reisbick, 1973; Koran et al, 1977). For coronal placement, this requirement is less stringent. Studies injecting light bodied polyvinyl siloxane material into prepared canals of extracted teeth indicate 100,000 centipoise is a maximum viscosity that can be reasonably inserted into the pulp space, e.g., by injection, without excessive force. An intact dentin-gel interface can be used in order to avoid microleakage, thus post-setting dimensional stability is significant. According to ADA Spec. No. 57 (American National Standard/American Dental Association Specification No. 57, 2000), the post-setting dimensional change can be less than 1% in contraction and 0.1% in expansion after 30 days. To test the suitability of a composition, both wet and dry conditions can be investigated.

The integrity of the gel over time is also important. Collagen, alginate, PEG-fibrinogen and chitosan can degrade in vivo. This can be a desirable feature of a construct. To test a matrix, 30 days can be used as an initial guide for gel integrity since pulp revascularization in avulsed teeth is reported to be complete within a month.

Compared to many other loose connective tissues, pulp structure has a more open and less fibrous architecture. There is evidence that the pulp itself can regenerate in the presence of non-viable pulp tissue in avulsed teeth. Therefore coronal pulp microstructure is a design mimicked by the compositions of the disclosed subject matter. The coronal pulp structure is more similar to developmental tissue than radicular pulp. Gel structure similar to that of coronal pulp can be more conducive to pulp regeneration. The less fibrous structure of pulp is modeled by varying the percentages of types I and III collagen. The gel mixture with the percentage closest to that found in pulp tissue can be used, such as about 45% type III and about 65% type I collagen. In addition, for pulp regeneration to occur, it is desirable that mineralization be confined mainly to the dentin surface. Some mineralization within the pulp is allowable since many teeth contain mineralized tissue within the pulp but the degree of mineralization can be controlled. The inclusion of type III collagen can aid in the control of non-specific mineralization. In addition, angiogenesis is enhanced in type III collagen versus type I collagen gels.

Unlike some other tissue-engineered constructs, a pulp construct is not required to provide a major role in the overall mechanical function of the tooth. However, during cell culture and in vivo placement, cellular attachment onto the matrix molecules exerts mechanical forces on the gel matrix. These mechanical forces cause significant contraction of the gel. (Brock et al, 2002) reported that cellular induced contraction also occurs with pulp cells. If the gel is constrained contraction does not occur and mechanical force is exerted to the cells. Since cells are affected by their mechanical environment, growth and differentiation differs on free and restrained gels. Therefore both free and restrained gels are encompassed by the disclosed subject matter. Also, since dimensional stability is desirable within 30 days of implantation, a matrix with higher stiffness can be more desirable. The mechanical properties of the pulp tissue engineered construct can play a role in regeneration.

Estimates of pulp properties for finite element analysis include 0.0 Mpa (Thresher, 1973), 0.003 Mpa (Toparli et al, 1999), and 2.0 Mpa (Williams et al, 1984). Two other desirable characteristics for pulp regeneration are angiogenesis and an infection free environment. The disclosed subject matter encompasses an optimal matrix capable of supporting pulp fibroblast growth and differentiation, while possessing structural and functional properties favorable for implantation.

### Pulp Regeneration

If a pulp regeneration tissue engineered construct is placed coronal to the viable pulp, it is possible to restore both the pulp and dentin closer to the pre-trauma form than with current techniques. With this method, not only would maximal tooth structure be regenerated, but the required traditional restoration would be minimal.

The composition and methods of the disclosed subject matter represent a paradigm shift in treating a traumatized pulp in a tooth. Instead of being removed, pulp tissue is encouraged to form a hard tissue barrier to seal the pulp space for regeneration of pulp to restore and/or replace injured pulp. In some aspects of the disclosed subject matter, stem cells, e.g., stem cells derived from pulp or cranio-facial sources, are used in a construct to promote regeneration of pulp. Regeneration of pulp has advantages over current techniques that are in general use. Regeneration can enable the continued full functioning of the tooth. The ability of the damaged tooth to self-repair, fight infection, and sense stimuli would be re-established. In short, in this proactive approach a tissue engineered construct is placed such that pulp can regenerate through the construct.

Two types of pulp constructs can be used. In the first type (termed herein, a “barrier construct”), the construct stimulates only pulp regeneration, and a barrier is placed where initiation of dentin formation is required. In the second type, an interfacial construct is fabricated with the apical portion designed for pulp regeneration and the coronal portion designed for dentin regeneration. This type of construct is termed herein an “interfacial construct.”

The potential health impacts of a tissue engineered pulp construct over current clinical treatments can include 1) reduce the present treatment-induced removal of sound hard tissue, thereby reducing the need for extensive restorative work, 2) provide a biological seal, thereby decreasing leakage and future ingress of bacteria and other noxious agents, 3) continue and/or improve dentin repair/regeneration, thereby reducing the need for re-treatment due to future pulp assaults, 4) continue and/or improve the pulp host immune system, thereby reducing post-treatment infections and the high costs of re-treatment, and 5) support the growth of immature teeth, thereby reducing the number of tooth fractures. In summary, a pulp construct can reduce the need for re-treatment procedures and increase the life span of the restored teeth.

### Pulp Tissue Engineering

A rigid resorbable construct such as polygylcolic acid polymer (Bohl et al, 1998; Bouvier et al, 1990; Mooney et al, 1996) can be utilized in pulp regeneration (similar to the use of gutta-percha points during root filling) for delivery of cells or cellular mediators. In such constructs a gel-like material is also required to fill the pulp space and thereby inhibit and/or prevent infection. This structure that includes the gel-like material with other components such as a rigid resorbable construct, cells, drugs, and growth factors is termed herein a “pulp construct.”

One aspect of the disclosed subject matter uses alginate, a hydrogel used in tissue engineering. In another aspect, the gel is a collagen or combination of collagens (e.g., type I and type III collagen). The gel can, in some cases, be chitosan or PEG-fibrinogen. Optionally, cells can be embedded in the gel during gelation, e.g., to produce a construct that can seed and populate the pulp space and promote pulp regeneration. The cells can be pulp cells, e.g., derived from cultures of pulp cells, or stem cells that can be derived, e.g., from a non-pulp source. Constructs that specifically include cells are referred to herein as “tissue engineered constructs.” Phenotypic expression of pulp cells can be manipulated in cell culture (Hao et al, 1997; Couble et al, 2000; About et al, 2000; About et al, 2002), therefore such manipulation can be utilized in the tissue engineered construct.

In some embodiments, a pulp construct includes a drug delivery system, e.g., to deliver at least one of an antibiotic, anti-fungal agent, angiogenic factor, cell growth factor, nerve growth factor, cell differentiation factor to the pulp space. An anti-inflammatory agent can be included in a construct. Another component of such constructs can be dentin powder. Such agents are known in the art. In some cases, the pulp construct includes chitosan (e.g., as the sole gel component of a pulp construct or as a portion of a pulp construct.

In constructs that include dentin powder, such powder can be obtained from the tooth being treated, e.g., by means of drilling, obtained from healthy teeth such as wisdom teeth, or from other preparations known in the art.

Delivery systems that can be included in a construct include polymer beads such as those known in the art and used for drug delivery. Non-limiting examples such polymers include poly-alpha-hydroxyester, poly-capralactone, polygalactins, polycyanoacrylates, polyphosphazenes, and polyanhydrides. Other drug delivery systems are known in the art and can be adapted for use in the present disclosed subject matter (e.g., U.S. Pat. No. 5,308,701, Richardson et al, 2001). Other drug delivery compositions known in the art can be used in certain constructs.

### Clinical Applications

In the dental tissue engineering methods described herein, several levels of regeneration are possible, any one of which can improve clinical outcomes. The optimal outcome level is the complete regeneration of pulp structure and function, including odontoblasts and complete innervation and revascularization of a damaged tooth. The next level is the restoration of dentin repair and immune response with incomplete regeneration of structure and function, e.g., incomplete reinnervation. Another outcome is the regeneration of pulp without the dentin repair. Finally, a vascularized non-pulp tissue can retain a host immune response. Each of the outcome levels represents improved clinical success since they can provide a biological seal, and thereby reduce the post-treatment infection rate. Accordingly, the pulp constructs provided herein can result in an improved or complete biological seal.

Non-limiting considerations for clinical application of a pulp construct include: 1) apical foramen, 2) effects on the dentin walls during anti-bacterial irrigation and cleaning of coronal pulp space, 3) attachment of the construct to dentin, 4) cell source, and 5) possible excessive mineralization in the pulp space. These are discussed in additional detail infra.

### Dental Apical Foramen

The apical foramen is a hole or holes at the apex of the root of a tooth through which nerves and blood vessels pass. The apical foramina are large at a young age, especially in the developing tooth, and decrease in size as the tooth grows older. Preservation of the apical foramen is therefore an important consideration in delivering a construct to the pulp space.

### Effect of Antimicrobial Agents

Antimicrobial agents are used to prepare a tooth for receiving a pulp construct. However, some antimicrobial agents are cytotoxic at concentrations in general use and can also adsorb to the dentin. Thus, when such an agent is used to prepare a tooth, the agent can be retained at concentrations that can be toxic to cells within the treated tooth. Examples of such agents include, without limitation, tetracycline, Metronidazole, Ciprofloxacin, Minicyclin and other agents known in the art. Thus, when antimicrobial agents are used as part of a treatment protocol for treatment using a pulp construct, such cytotoxic effects must be reduced. Methods of assaying cytotoxic effects are known in the art. Accordingly, in some cases, an antimicrobial agent or combination of agents that have relatively low cytotoxicity are selected for use with a pulp construct. Alternatively, an antimicrobial agent is selected for its low dentin adsorption properties. Examples of such agents include, without limitation, Ciprofloxacin and Metronidazole. In some methods, an antibiotic local drug delivery system is located within the pulp construct. Examples of such local delivery systems include incorporation of one or more antibiotics into a matrix for extended release, e.g., using polymer microspheres as described herein and as are known in the art.

The use of a pulp construct can include treatment and shaping of dentin walls, which can be beneficial to the success of the tissue engineered construct, for example, because the treatment of dentin walls can cause the release of growth factors that can aid in regeneration.

### Attachment of a Pulp Construct to Dentin

An attachment aids in interfacial integrity during possible setting shrinkage, construct contraction by cells, and scaffold degradation. Cells growing on extracellular matrices exert a force on the extracellular matrix molecules. These cellular forces cause the matrix to contract. The contraction is significantly larger than the contraction during polymerization. Agents attaching/securing the gel to dentin can be used to reduce the gel contraction in the presence of cells. Total contraction can also be reduced by the addition of points constructed with polyglycolic acid or its composites (PGA). PGAs are appropriate since pulp cells grow well on PGA (Bohl et al, 1998; Bouvier et al, 1990; Mooney et al, 1996). Also, PGAs can be used as a drug delivery system for angiogenesis and antibiotics (Bouhadir et al, 2001; Peters et al, 2002). The points can reduce the volume of gel required and therefore the total overall contraction. Gel contraction can also be controlled by varying gel microstructure, as well as cell culture duration time within the gel before injection.

### Cell Source

Pulp cells for compositions and methods described herein can be from autogenous or allogenic sources. Alternate sources include expanded cells from the injured tooth, donor pulp cells, stem cells, or host transduced cells. In addition, a construct without cells can be sufficient for pulp regeneration. Other cell types that can be used include embryonic stem cells, mesenchymal cells, umbilical cord-derived cells, stem cells of bone origin, and stem cells of cranio-facial origin. Methods for obtaining stem cells including pulp-derived stem cells, are known in the art (Gronthos et al, 2002; Gronthos et al, 2000).

### Mineralization

Ectopic mineralization in the tissue-engineered construct can occur and hamper pulp regeneration. Calcified structures occur in teeth—as high as 50% of newly erupted teeth and 90% of older teeth can contain calcified nodules. Although these calcifications are generally non-problematic, their presence indicates that uncontrolled calcification can occur during pulp regeneration. Nonfunctional calcification can be controlled by adjusting the construct material to one that does not favor calcification or by spatially varying the construct materials as well as cellular mediators.

In young teeth damaged by trauma or caries, the coronal and cervical portions of the pulp can be necrotic or severely damaged. Even in these cases, there can be vital pulp tissue remaining in the damaged tooth that is capable of healing in the root canal. The disclosed subject matter thus provides a novel approach to obtain pulp and dentin regeneration in young teeth in which viable pulp is situated apically to the levels where pulp capping and partial pulpotomy would be performed. A restoration of pulp function in the cervical and coronal areas restores dentin formation thereby increasing the hard tissue thickness. This inhibit and/or prevents cervical fractures, which are common in young teeth that were damaged before cervical hard tissue formation has reached sufficient thickness to reduce the chance of such fractures. A decrease in the restorative needs in such pulp-dentin restored teeth is achieved by the methods and compositions of the disclosed subject matter.

In some embodiments of the disclosed subject matter, a pulp construct is combined with other more generally used methods of tooth repair. For example, the area incisally/occlusally located with respect to a placed pulp construct is closed to the oral cavity with composite resin bonded to surrounding enamel. This provides a temporary seal, until a dentin bridge is formed, and fulfills esthetic needs of patient.

### Features Tested to Identify Compositions

The disclosed subject matter includes an injectable hydrogel matrix with appropriate viscosity, dimensional stability, microstructure, and mechanical properties for pulp regeneration. In general, the hydrogel matrix is based on one of three types of hydrogel-based materials, collagen type I, combined matrices of collagen types I and III, chitosan, and alginate. The disclosed subject matter encompasses methods to evaluate the suitability of these three types of hydrogel-based materials from structural and functional perspectives.

TABLE 1

| Experimental | | |
|---|---|---|
| Variables | Composition | Fabrication |
| Type I Collagen | 0.3 mg/ml, 1.5 mg/ml, | Gelation pH (6, 7.5, 9) |
| | 3.0 mg/ml | |
| Type I and III | I:III Ratios - 30:70, | Gelation pH (6, 7.5, 9) |
| Collagen | 55:45, 70:30 | |
| Alginate | 1%, 3%, 5% (wt %) | CaCl₂ concentration (e.g., |
| | | 5%, 10%, 20% (w/v %)) |
| Chitosan | 1%, 3%, 5% (wt %) | Degree of acetylation (e.g., |
| | | 70%, 80%, 90%, 95%) |
| PEG-fibrinogen | About 10% w/v | Additional PEG-diacrylate |
| | | 1%, 2%, 3%, |
| | | 4% (wt %) |

The methods encompass measuring the structural and functional properties of the resultant matrices generated by controlling the parameters listed in Table 1. Specific examples of such structural and functional properties include morphology, porosity, fiber organization, dimensional stability, viscosity, and mechanical properties under confined compression. Table 2 provides additional detail as to the features of these structural and functional properties that can be used to indicate the suitability of a cell or cells cultured by a selected culture method.

TABLE 2

| | Type of Pulp Matrix | | | | |
|---|---|---|---|---|---|
| Matrix | Types I | Types I and | | | PEG- |
| Properties | Collagen | III Collagen | Alginate | Chitosan | fibrinogen |
| Structural | 1. | 1. Morphology | 1. Surface | 1. Morphology | 1. Morphology |
| | Morphology | (by SEM) | morphology | (by SEM) | (by SEM) |
| | (by SEM) | | and overall | | |
| | | | matrix | | |
| | | | organization | | |
| | | | (SEM) | | |
| | 2. Porosity | 2. Porosity (by | 2. Porosity | 2. Porosity (by | 2. Porosity (by |
| | (by | quantitative | (by | quantitative | quantitative |
| | quantitative | image | quantitative | image | image |
| | image | analysis) | image | analysis) | analysis) |
| | analysis) | | analysis) | | |
| | 3. Fiber | 3. Fiber | | | |
| | organization, | organization, | | | |
| | fiber | fiber diameter, | | | |
| | diameter and | and length (by | | | |
| | length (by | SEM) | | | |
| | SEM) | | | | |
| | | 4. Integration | | | |
| | | of I&III | | | |
| | | collagen (by | | | |
| | | light and | | | |
| | | SEM) | | | |

| | |
|---|---|
| Functional | 1. Dimensional stability (% contraction) in wet & dry conditions. 2. Viscosity |
| | 3. Mechanical properties under confined compression |

### Construct and Matrix Fabrication

Matrices are compared for their suitability as matrices for growth of pulp fibroblasts, e.g., for promoting cell proliferation and/or differentiation and for implantation purposes. In some cases, a construct includes a gel but does not include a rigid component. One such construct includes alginate, a biocompatible, degradable biomaterial widely used in soft tissue engineering. The matrix can be is compared in surface, structural, and mechanical properties with other matrices containing, e.g., type I collagen, as well as composites of types I & III collagen matrices. These collagen types are selected based on features of native pulp tissue, which is comprised of approximately 55% type I collagen and 45% type III collagen (van Ameronsen et al, 1983). For example, different alginate gels can be fabricated by varying the alginate concentration (e.g., 1%, 3%, or 5% (w/v)) and CaCl₂ concentration (for example, 50 mM, 100 mM, or 200 mM CaCl₂). Alginate gels are formed by mixing alginate solution (Sigma, St. Louis, Mo.) with a known concentration of CaCl₂ solution in a rectangular mold. After the gel has set (about 15 minutes), discs of pre-determined size are corked from the rectangular gel and used for culturing cells. Both type I (0.3 mg/ml, 1.5 mg/ml, and 3 mg/ml final concentration in the gel) and type I and type III composite collagen gels are also useful. The polymerization variables for assays used to select suitable constructs are the collagen solution concentrations and gelation pH (pH 6, pH 7.5, pH 9). Briefly, in these experiments, collagen (Invitrogen) is first dissolved in 0.01 M HCl, and gelation occurs after neutralizing the collagen solutions in 10×PBS and incubation at 37° C. The type I and type III composite collagen gels are manufactured by combining type I and type III collagen in various ratios (for example, I:III wt % ratios of 30:70, 55:45, and 70:30), and gelation following the method described supra.

Chitosan and PEG-fibrinogen gels are fabricated by cross-linking using methods known in the art. In general, gels for a pulp construct can be made in the presence of other components of the construct such as beads containing one or more drugs, growth factors, or differentiation factors.

### Construct and Matrix Characterization—Structural and Functional Properties

For a gel matrix, surface morphology and overall matrix organization is determined as a function of the variables listed in Table 1. The surface morphology, matrix organization, fibril number and diameters are examined via scanning electron microscopy (SEM, JEOL 5600LD, 5 keV), confocal microscopy, and phase contrast microscopy. Organization in the gel interiors or cross sections can be also be examined by SEM. Matrix porosity is determined via quantitative image analysis of the SEM and light microscopy images using the Zeiss Axiovision modular image analysis package (Axiovision 3.1, Zeiss). The fibril density, diameter, and length of the type I and type I and III collagen gels is examined via confocal microscopy (Roeder et al, 2002) and quantitative image analysis. Other methods known in the art for visualizing a matrix and assessing structural and functional properties can be used.

### Dimensional Stability

Dimensional stability is the ability of a substance (e.g., matrix) to resist changes caused by environmental factors. In the case of a matrix as used herein, dimensional stability relates to dimensional changes following gelation. Dimensional stability is measured following the methods described in the American Dental Association (ADA) Specifications No. 57; the methods described in the specifications can be applied to the matrices described herein as standardized techniques to measure gel properties.

Gel viscosity can also be measured, e.g., using an Ubbelohde viscometer (Cannon Instrument Co., State College, Pa.). Kinematic viscosity is calculated by multiplying the efflux time by the viscometer constant. Viscosities are measured for, e.g., 0 minutes, 5 minutes, and 15 minutes.

### Functional Properties

Functional properties of the gels are also examined in relation to matrix compositional and fabrication parameters. Although the mechanical properties of pulp tissue have not been well defined, and there are limited publications on the effects of mechanical stress on pulp and dentin regeneration, the mechanical properties of the hydrogel are directly related to its dimensional stability. Therefore, gels are tested under conditions of confined compression to determine their mechanical properties. Specifically, the mechanical properties of the gels are determined in confined compression using the method of Mauck et al, 2000.

In addition, the modulus and compressive strength of the hydrogel matrices can be compared to human pulp tissue tested under identical conditions to produce a matrix that mimics the properties of human pulp. For example, coronal pulp tissue is frozen and later shaped into approximately 3 mm wide cubes, and the mechanical properties of the coronal pulp tissues are measured along the long axis of the tooth as well as the two perpendicular axes. A confined compression test can utilize a rigid-porous permeable sintered steel indenter. The testing system consists of a computer controlled stepper micrometer displacement actuator, a linear variable displacement transducer (LVDT) to measure strain, and a load cell to measure stress. Before testing, free swelling disk thickness is measured with a current sensing micrometer. With a ramp speed of 1 μm/s, deformations on the order of 10%, 20%, and 30% are applied. For each deformation, stress-relaxation curves (force vs. time) are recorded utilizing a 10 g load cell. The equilibrium modulus is determined for each applied deformation, based on the equilibrium force determined from stress-relaxation curves.

### In Vitro Testing of Gel Matrices

To further test the properties of a gel and to identify a gel that is useful for culturing pulp fibroblasts, in vitro responses of pulp fibroblasts are assayed in a test gel, e.g., cell proliferation and differentiation of cells grown in contact with the matrix are tested. In addition, the effect of cell culture on the properties of the gel matrix are assayed (e.g., gel contraction).

The biocompatibility, and the potential of the a matrix to support the growth and differentiation of pulp fibroblasts can be examined, e.g., to identify a matrix suitable for culturing pulp fibroblasts and to serve as a matrix for transplanted pulp fibroblasts. In some embodiments, a composite matrix of type I and type III collagen is used as it closely mimics the native composition of the human pulp and can support cell proliferation and differentiation. Table 3 provides non-limiting examples of types of matrices, and the seeding density and culture duration that can be used to assess the ability of a matrix to promote cell proliferation and differentiation.

TABLE 3

| | Type of Optimized Pulp Matrix | | | | |
|---|---|---|---|---|---|
| Experimental | Types I | Types I and | | | PEG- |
| Variables | Collagen | III Collagen | Alginate | Chitosan | fibrinogen |
| Seeding | 1 × 10⁶, | 1 × 10⁶, | 1 × 10⁶, | 1 × 10⁶, | 1 × 10⁶, |
| Density | 2 × 10⁶, | 2 × 10⁶, 3 × 10⁶ | 2 × 10⁶, 3 × 10⁶ | 2 × 10⁶, 3 × 10⁶ | 2 × 10⁶, 3 × 10⁶ |
| (cells/ml) | 3 × 10⁶ | | | | |
| Culture | 1, 3, 7, 14, | 1, 3, 7, 14, | 1, 3, 7, 14, | 1, 3, 7, 14, | 1, 3, 7, 14, |
| Duration | 21, 28 days | 21, 28 days | 21, 28 days | 21, 28 days | 21, 28 days |

The effect of the above two parameters on the proliferation and differentiation of pulp-fibroblasts can measured as detailed in Table 4.

TABLE 4

| | | | |
|---|---|---|---|
| Culturing | | Type I & | |
| Parameters | Type I Collagen | Type III Collagen | Alginate |

| | |
|---|---|
| Proliferation | DNA Content (quantitative fluorimetric assay) |
| Differentiation | Synthesis-osteocalcin, alkaline phosphatase, types I |
| | and III collagen, dentin sialoprotein, osteopontin. |
| | (measured by quantitative and qualitative assays) |
| | Expression of dentin sialophosphoprotein & osteocalcin |
| | (measured by RT-PCR) |

Other methods known in the art can be used to assay markers of cell proliferation and pulp fibroblast differentiation. Cell responses and hydrogel matrix properties can also be evaluated in vitro. Such cell-related outcome measures can include cell morphology and 3-dimensional structure formation (measured by electron microscopy, Immunohistochemistry, immunofluorescence and histology), matrix synthesis exemplified in total collagen production measured by hydroxyproline assay (Lu et al, 2005), total glycosaminoglycan (measured by Blyscan GAG assay (Lu et al, 2005), alkaline phosphatase (ALP) activity (measured by colorimetric assay (Lu et al, 2003), and mineralization (measured by quantitative Alizarin Red assay (Lu et al, 2003b). In addition, matrix distribution within the hydrogel can be assessed histologically via Immunohistochemistry (Lu et al, 2005, Lu et al, 2003b). (collagen types I and III, dentin sialoprotein [DSP] and osteopontin). The scaffold-related outcome measures can include gel geometry (Tran-Hung, 2003) (diameter/height), wet and dry weight, as well as mechanical properties (Teng et al, 2005; Salani et al, 2000) as a function of gel concentration, cell seeding density and in vitro culture. Changes in gel geometry or gel contraction can be determined via quantitative image analysis methods (Teng et al, 2005). Gel mechanical properties (compressive modulus and yield strength) under applied compression can be determined. These values can be compared to that of the as-isolated native pulp tissue tested under similar conditions.

In addition to the cell-related outcome measures described above, angiogenesis with the hydrogel can be determined in terms of the formation of tubular structures as well as positive staining for endothelial membrane protein CD-31 and von Willebrand's factor (vWF) in order to identify vessels lined with endothelial cells. The formation of tubular structures due to PRP stimulation can be quantified using digital image analysis, by measuring the perimeter, area, or number of branches observed in the network following published methods (Tran-Hung, 2006; Salani et al, 2000). In addition, the production of VEGF, PDGF and bFGF by pulp cells can be determined via immunohistochemistry, and the concentration of these angiogenic factors in the cell culturing media can also be measured by ELISA (Tsay et al, 2005).

### Establishment of Human Pulp Fibroblast Primary Cultures

In some cases, the pulp constructs and methods for use of such constructs include the use of pulp fibroblasts. Accordingly, methods of culturing such fibroblasts for use for pulp constructs and for testing certain features of a pulp construct are encompassed by the present disclosed subject matter.

To culture human pulp fibroblasts, non-carious premolars and third molars from healthy individuals are collected, e.g., from surgical waste. Tooth surfaces are washed with 70% ethanol and the pulp is extracted after cracking the teeth. The pulp is washed five times with wash solution (Dulbecco's Modified Eagles Medium (DMEM), supplemented with 2% penicillin (10,000 IU)-streptomycin (10 mg/ml) solution, and 5.0 μg/ml of amphotericin B (Sigma, St. Louis, Mo.)). The pulp is cut into cubes with approximately 2 mm edges with a sterile surgical blade and placed inside a T-75 flask with a few drops (about 3-5 ml) of the above wash solution. To insure that the pulp pieces attach to the bottom of the flask, the flask is then placed in an incubator for 45 minutes. After attachment, a minimal amount of explant media (wash solution+10% FBS) is added to the flasks. Cellular outgrowth is monitored after 10 days of incubation. At least about 50 cells is considered sufficient outgrowth. If there is sufficient outgrowth, explant medium is added to bring the medium volume to approximately 13 ml. Once colonies form around the explants (a few hundred cells; about day 14) the medium composition is changed from the above explant medium to an explant medium containing half the concentration of pen/strep and antifungal agent. The medium is then changed every other day with 13 ml of medium. The time required to reach confluency depends on the number of attached pulp explants in the T-75 flask, but generally occurs about six weeks after tooth extraction.

### Cell Culture and Seeding on Optimized Hydrogel Constructs

Primary human pulp fibroblasts are cultured in DMEM+10% FBS and 1% penicillin (10,000 IU)-streptomycin (10 mg/ml) solution, and 2.5 μg/ml of Amphotericin B, at 37° C. and 5% CO₂ until confluence. First passage cells are embedded in the hydrogels and cell growth and differentiation is examined as a function of gel type, seeding density, and culturing duration.

To embed the cells in collagen, cell suspensions are added to collagen solutions after neutralization, but before polymerization. Briefly, 1.5 ml of pulp cell suspension in DMEM is mixed on ice with 5.0 ml of 3.1 mg/ml collagen solution (Vitrogen), 0.5 ml HEPES (25 mM), and 0.5 ml DMEM. Examples of final cell seeding densities and collagen concentrations are listed in Tables 2 and 1 respectively. The collagen/cell solution is then poured into a square mold inside a petri dish. The dish is incubated for 2 hours at 37° C. to allow polymerization to occur. The collagen mold system utilized was developed by Holmes et al. (Holmes et al, 2002). Briefly, a 4 cm×4 cm square mold (inner dimensions) is housed in a 100 mm×15 mm petri dish. Porous polyethylene bars (2 cm×5 mm×3 mm) are placed on the edges of the inner mold. Sutures are placed around the bars. The collagen mixture is poured into mold. After polymerization the mold is removed leaving a square collagen gel. For restrained gels, the sutures are taped to the petri dish thereby preventing contraction of the gel. For unrestrained gels the sutures remain unattached.

Cells are embedded in alginate by first combining fibroblasts in alginate solution. The gelation procedure is described herein.

### Characterization of Human Pulp Fibroblast Response to Optimized Hydrogel Constructs—Cellular Attachment and Growth Morphology

To assay the effect of a hydrogel composition on human pulp fibroblasts, cellular attachment and growth morphology are be examined. For example, the characteristics of attachment and growth morphology can be assayed using histological staining and scanning electron microscopy (SEM, JEOL 5600LD, 5 keV). Non-adherent cells are removed by washing cultured pulp fibroblasts at selected time points, e.g., the samples are washed three times with PBS to remove non-adherent cells.

For cytochemical staining, samples are fixed in 4% paraformaldehyde, dehydrated in ethanol and embedded in paraffin. The samples are then sectioned and stained with hematoxyline and eosin using methods known in the art. For SEM analysis, the samples are first dehydrated using an ethanol drying series, and then left to dry in Freon overnight in a chemical hood. Prior to imaging, the samples are coated with carbon to eliminate charging effects. Cell proliferation is determined using the PicoGreen® dsDNA quantitation assay (Molecular Probes, Carlsbad, Calif.) where fluorescence intensity is correlated with DNA concentration. In general, it is desirable that cultured pulp cells have features of proliferating cells and the capacity to express proteins indicative of further development to become functional pulp cells.

### Phenotype Assays

Assays can be performed to ascertain the phenotype of the cultured pulp fibroblasts. In general, a cultured pulp fibroblast that is suitable for use in the disclosed subject matter (i.e., for implantation) exhibits at least one of the following, alkaline phosphatase synthesis, osteocalcin production, dentin sialophosphoprotein, or both types I and III collagen synthesis. Immunofluorescent staining can be used to qualitatively examine the expression of these proteins by these cells following the methods of Gronthos et al. (Gronthas et al, 2000; Gronthas et al, 2002). Alkaline phosphatase expression is quantified using a colorimetric assay. In such assays, a sample is incubated at 37° C. for 30 minutes in 0.1 M Na₂CO₃ buffer containing 2 mM MgCl₂ with disodium p-nitrophenyl phosphate (pNP—PO₄) as the substrate. Standard solutions are prepared by serial dilutions of 0.5 mM p-nitrophenol (pNP) in Na₂CO₃ buffer. Enzymatic activity is expressed as the total nmoles of pNP produced per minute per total cell number. Absorbance is measured at 415 nm using a Spectrofluor reader (Tecan). The synthesis of type I and III collagen by the human osteoblast-like cells is quantified using a modified ELISA assay used by Lu et al (Barthel et al, 2000). The expression of osteocalcin and dentin sialophosphoprotein (DSPP) are detected by reverse transcription followed by polymerase chain reaction (RT-PCR). For this purpose, cells are released from alginate by standard procedures using sodium citrate, and from collagen gels using collagenase digestion. Cells can be released from chitosan using methods known in the art, e.g., chitinase digestion. Total RNA is isolated using Rneasy® kit (Qiagen, Valencia, Calif.). First strand cDNA is synthesized using Superscript (Invitrogen). PCR is performed using the following primer sets: osteocalcin, sense 5′-CATGAGAGCCCTCACA-3′ (SEQ ID NO: 1) and antisense 5′-AGAGCGACACCCTAGAC-3′ (SEQ ID NO:2); DSPP, sense 5′-GGCAGTGACTCAAAAGGAGC-3′ (SEQ ID NO:3) and antisense 5′-TCATATTTGGCAGGTTTTTCT-3′ (SEQ ID NO:4) (Gronthos et al, 2002; Gronthos et al, 2000). PCR is performed for 35 cycles at annealing temperature of 56° C. PCR products are analyzed using 1.5% agarose gel electrophoresis and visualized by staining with ethidium bromide.

In some cases, the formation of a mineralized matrix by the cultured pulp fibroblasts is determined. Although pulp fibroblasts do not generally exhibit mineralization under physiological conditions, the ectopic formation of mineralized nodules by these cultures in the optimized matrices is examined by SEM/EDXA, and, if necessary, the specific Ca/P ratio IS calculated based on a hydroxyapatite standard. Mineralization can be further confirmed using Alizarin Red S (ALZ) staining specific for calcium. The samples are washed in double distilled H₂O, and incubated in 40 mM Alizarin red solution for 10 minutes. After additional washes, the scaffolds are incubated in 10% cetyl pyridinium chloride for 15 minutes to solubilize reacted ALZ. In this assay, serial dilutions of 1 M CaCl₂ are used as standards. ALZ concentration per cell is calculated as molar equivalent CaCl₂ divided by the average cell number. Absorbance is measured at 570 nm using a Tecan Spectrofluor system (Tecan, Durham, N.C.).

Other methods known in the art can be used to assay the marker proteins or RNAs. The expression of these proteins in cultured pulp fibroblasts indicates that such cells are suitable for use in implantation, e.g., to repair a tooth. In general, suitable conditions for culturing a pulp cell are those in which at least 80%, e.g., at least 90% of cells express one or more pulp cell markers.

### Transplantation and Treatment of a Damaged Tooth

To administer a construct to the pulp space, the entire treatment procedure is performed under aseptic conditions with the use of a rubber dam (to keep saliva, etc. away from the treatment site), sterilization of the field of work, and the use of sterile instruments. Treatment includes at least the following; 1. Mechanical removal of caries that can be present; 2. mechanical removal of infected and damaged pulp tissue that can be present; 3. irrigation of the treatment site with an antibacterial solution; 4. treatment of dentin surfaces with e.g., EDTA; 5. placement of a construct; and 6. sealing of the treatment area.

### Animal Models

Constructs can be tested using animal models for tooth damage, e.g., canine models (e.g., Skoglund and Hasselgren, 1992; Skoglund, 1983; Hasselgren et al, 1977). Methods used for implantation of a construct are described herein and methods are known in the art.

### Data Analysis

For data analysis, primarily two-way analysis of variance (ANOVA) is performed to determine any statistically significant relationship between factors examined in the proposed experiments. Once a significance difference is predicted by ANOVA, the Tukey-Kramer significance test is used to compare between the group means. Statistical significance is tested at p<0.05. Statistical analysis can be performed using the Sigma Stat statistic software (SPSS) or other suitable software.

The specific variables that are measured include porosity, dimensional stability, viscosity, mechanical properties, as well as fiber diameter and length. The factors are percent gel material in the composition, e.g., the percent of collagen, alginate, or chitosan in solution and either CaCl₂ concentration for alginate or pH for collagens. The effect of gel composition can also be analyzed.

The specific variables to be measured generally include light and SEM image analysis of cells, alkaline phosphatase expression, DNA synthesis, osteocalcin expression, and dentin sialophospho-protein expression. The factors are culture duration and seeding density.

### Pulp Regeneration

If a pulp regeneration tissue engineered construct is placed coronal to the pre-trauma pulpodentin interface, it is possible to restore both the dentin and the pulp closer to the pre-trauma form than with current techniques. With this method, not only is maximal tooth structure be regenerated but the required traditional restoration is minimal (see FIGS. 1A-1E). Although some dental material may be required, regeneration is enhanced and tooth life expectancy increased. The restored tooth is closer to its original form. In some cases, such methods can decrease the clinical technical complexity.

A pulp regeneration tissue engineered construct includes at least a gel matrix (e.g., a collagen matrix, or a hydrogel such as an algenate matrix or a chitosan matrix), and optionally, pulp fibroblasts. In addition, the construct can include one or more of an antibiotic, an antifungal agent, or a growth factor (including one or more factors that promote cell proliferation and/or differentiation). The disclosed subject matter represents a paradigm shift in treating traumatized pulp. Instead of being removed, pulp tissue is induced to regenerate. In some cases, the regenerating pulp can form a hard tissue barrier in order to seal the pulp space. Regeneration of pulp has several advantages over current techniques for treating a damaged tooth. Regeneration enables the continued full functioning of the tooth. The ability of a damaged tooth to self-repair, fight infection, and sense stimuli can be re-established using pulp regeneration methods as described herein. In short, in this proactive approach, a tissue engineered construct through which pulp can regenerate is placed within a tooth.

Two types of constructs are provided herein. In the first, the construct stimulates only pulp regeneration and a barrier is placed where initiation of dentin formation is required. This type of construct is referred to herein as a “simple pulp regeneration construct.” In the second type, an interfacial construct is fabricated with the apical portion designed for pulp regeneration and the coronal portion designed for dentin regeneration. This type of construct is referred to herein as an “interfacial construct.”

### Possible Health Impact of Tissue Engineered Aided Pulp Regeneration

The potential positive health impacts of a tissue engineered pulp construct over current clinical treatments include 1) reduction in the present treatment-induced removal of sound hard tissue, thereby reducing the need for extensive restorative dental work, 2) provides a biological seal decreasing leakage and future ingress of bacteria and other noxious agents, 3) continue and/or improve dentin repair/regeneration, thereby reducing the need for re-treatment due to future pulp assaults, 4) continue and/or improve pulp host immune system reducing post-treatment infections and the high costs of re-treatment, 5) support the growth of immature teeth thereby reducing the number of tooth fractures. Therefore, successful utilization of such a construct can reduce the need for re-treatment procedures and increase the life span of the restored teeth.

### Design of Tissue Engineering of Pulp Constructs

The entire pulp space must be filled with the tissue engineered construct for optimal control of regeneration and to inhibit and/or prevent infection. To this end, the gel can have sufficient viscosity and volume to be injected into the entire space. Dimensional stability before degradation is desired. Factors affecting stability are setting contraction, contraction due to applied forces by cells, thermal cycling contraction, and premature degradation. The methods described in ADA Spec. No. 57 can be used as a standard to measure dimensional stability. An intact dentin construct interface is important to avoid microleakage. A pulp regeneration time of thirty days can be used as an initial guide for construct degradation. Varying composition, molecular weight, and microstructure can control these properties. The presence of cells can affect dimensional stability and degradation. Therefore these two properties must be re-measured in the presence of cells. It is unclear to what extent construct mechanical properties may play a role in pulp regeneration. Unlike some other tissue engineered constructs, the pulp construct is not required to provide mechanical support in order for the tooth to function. However, mechanical properties can play a role in the pulp tissue construct's success. For example, the presence of pulp cells can cause significant additional contraction of a three dimensional gel. Since dimensional stability is a is important, a stiffer gel than that used in that particular study can be used. In addition, the mechanical environment affects the behavior of many cell types. Therefore, the mechanical properties of the pulp tissue engineered construct can play a role in regeneration.

### Clinical Application

In any dental tissue engineering application, it is possible to have several levels of regeneration and still improve clinical outcomes. The optimal level is the complete regeneration of pulp structure and function, including odontoblasts and complete innervation and revascularization. The next level is the restoration of dentin repair and immune response but an incomplete regeneration of structure and function. A third possibility is the regeneration of pulp without the ability to repair dentin. The final possibility is the regeneration of a vascularized non-pulp tissue providing both a biological seal against microleakage, as well as retaining a host immune response. Thus, the methods of the disclosed subject matter can improve clinical success since they can provide a biological seal, thereby reducing the post-treatment infection rate.

Possible challenges to clinical application include: 1) reduced apical foramen size in mature teeth, 2) effects of traditional endodontic treatment on the dentin walls during anti-bacterial irrigation and cleaning and shaping with root canal instruments, and 3) source of cells. If the apical foramen width is narrow, revascularization does not occur. For pulpectomy treated teeth with narrow foramina the apex can have to be widened. Although such widening is possible since canals are shaped during endodontic treatment, widening the apical foramen without damaging the periradicular bone and ligament can present technological issues.

Treatment of the dentin walls with currently used antimicrobial agents can be evaluated for cytotoxicity since such agents can be cytotoxic at the commonly used concentrations. These agents can initially be adsorbed into the dentin and released with time. Antimicrobial agents that are less cytotoxic or that do not readily adsorb into the dentin can be utilized. Such agents can be identified using the culture methods described herein and testing an agent for efficacy and cytotoxicity using methods known in the art. Also the inclusion of an antibiotic local drug delivery can reduce the level of antimicrobial required. The treatment and shaping of dentin walls can actually be beneficial to the success of the tissue engineered construct. Finally, the treatment of dentinal walls can cause the release of growth factors that can aid in regeneration. Also, dentin treatment can allow the use of dentin bonding agents to increase the strength of the interfacial bond between construct and dentin. A stronger bond would aid in interfacial integrity during possible setting shrinkage, oral thermal cycling, construct contraction by cells, and scaffold degradation.

A large number of cells can be needed in the repair of critical size defects such as after pulpectomy. At the present time the use of pulp cells from autogenous or allogenic sources are highly impractical. Alternate sources to be considered include expanded cells from the injured tooth, donor pulp cells, stems cells, or host transduced cells. In addition, ectopic mineralization in the tissue engineered construct can occur and disrupt the path to pulp regeneration. Calcified structures are fairly common, as high as 50% of newly erupted teeth and 90% of older teeth can contain calcified nodules. Although these calcifications are normally non-problematic, their presence indicates uncontrolled calcification can occur during pulp regeneration. Nonfunctional calcification can be controlled by adjusting the construct material to one that does not favor calcification or by spatially varying the construct materials as well as cellular mediators.

In general, after a pulp construct is placed within the pulp space of a tooth, it is important to provide at least a temporary sealant. Such sealants are known in the art, e.g., a composite resin that can be bonded to surrounding tooth structure. The features of such a sealant include, when set, biocompatibility and bonding properties that are maintained for a sufficient amount of time for the pulp to recover and regenerate a seal.

The disclosed subject matter encompasses an injectable hydrogel matrix with appropriate viscosity, dimensional stability, microstructure, and mechanical properties for pulp regeneration. An evaluation the suitability of three types of hydrogel-based materials (chitosan, alginate, collagen type I, and a combined matrix of collagen type I/III) from a structural and functional perspective is provided.

Compositions as described herein can be provided in kits suitable for preparation of a construct and treatment of a damaged tooth by a practitioner. Kits can be for treating specific conditions, e.g., for treating a tooth with remaining pulp apically, a tooth without living pulp cells (where the kit has to provide e.g. stem cells). A kit can also contain components needed for a practitioner to collect a pulp cell sample from a patient (e.g., by providing a culture container and medium with suitable components such as antibiotics), which is sent to a laboratory for preparation of a construct.

### Construct Fabrication and Optimization

Alginate gels can be fabricated by manipulating two parameters, alginate concentration (for example, 1%, 3%, or 5% (w/v)) and CaCl₂ concentration (5%, 10%, 20% (w/v) CaCl₂). Alginate gels by mixing alginate solution (Sigma) with CaCl₂ solution. The alginate solution can be dropped into a stirred CaCl₂ solution. Spherical beads are formed during gelation. Beads are removed from solution after 60 minutes and washed in distilled water.

Several different collagen gels can be fabricated, for example, gels containing a single type of collagen, as well collagen mixtures with type I:type III ratios of 30:70, 55:45, and 70:30. The polymerization variables are the collagen solution concentrations (0.3 mg/ml, 1.5 mg/ml, 3 mg/ml final concentration in the gel) and the pH (6, 7.5, 9). Collagen is dissolved in 0.01M HCl. Gelation occurs by neutralizing the collagen solutions in 10×PBS and placing in a 37° C. incubator. Total phosphate ionic strength is held constant at 0.14 M.

Chitosan and PEG-fibrinogen gels can be formulated using methods known in the art and illustrated in the Examples (infra).

Gel microstructure and porosity is determined via image analysis. For alginate the beads are cryo-fractured and the surface and interior examined with SEM. For the collagen gels the fibril density, diameter, and length is determined via confocal microscopy (Rungvechvottivittaya et al, 1998), although SEM can also be used. Chitosan gels can be examined using confocal microscopy or SEM. Other suitable methods for examining gel morphology and known in the art can also be used for such examinations.

Dimensional stability is analyzed using ADA Spec. No. 57 as a guide. Measurements are made in both dry and wet conditions at 37° C. for 1 day, 2 days, 7 days, 14 days, 21 days, and 28 days. For wet conditions the gel is placed on a Millipore filter. This filter is placed on a cylindrical support and put into a petri dish. DMEM is added to the dish up to the height of the filter paper.

The Ubbelohde viscometer is used to measure kinematic viscosity for all gels. Kinematic viscosity is calculated by multiplying the efflux time by the viscometer constant. Viscosities are measured for various times, e.g., 0 minutes, 5 minutes, 2 hours, 10 hours, and 3 days.

Gels are tested in confined compression to determine their mechanical properties (Ranley et al, 2000). The modulus is acquired from the equilibrium force determined from stress-relaxation curves. Confined compression test utilizes a rigid-porous permeable sintered steel indenter. The unconfined compression test utilizes a rigid-impermeable glass loading platens. Before testing free swelling disk thickness is measured with a current sensing micrometer. The custom designed testing system consists of a computer controlled stepper micrometer displacement actuator, an LVDT to measure strain, and a load cell to measure stress. With a ramp speed of 1 μm/s deformations of, e.g., 10%, 20%, and 30% are applied. For each deformation stress-relaxation curves (force versus time) were recorded utilizing a 10 g load cell. The equilibrium modulus was determined for each applied deformation.

The disclosed subject matter provides methods to modulate the proliferation and differentiation of pulp fibroblasts in the biodegradable gel matrix. The structural and functional similarities to native pulp tissue, the ability of the experimental matrices to support the growth and differentiation of pulp fibroblasts are examined. The composite matrix of type I collagen and type III collagen is one useful matrix because it mimics the native composition of the human pulp and supports cell proliferation and differentiation.

The following examples illustrate the present disclosed subject matter, and are set forth to aid in the understanding of the disclosed subject matter, and should not be construed to limit in any way the scope of the disclosed subject matter as defined in the claims which follow thereafter.

### EXAMPLES

The disclosed subject matter is further illustrated by the following examples. The examples are provided for illustrative purposes only. They are not to be construed as limiting the scope or content of the disclosed subject matter in any way.

### Example 1

### Establishment of Pulp Fibroblast Cultures

A protocol for establishing human pulp cells from explant outgrowth has been developed and is described herein. For preliminary purposes, the outgrowth of porcine pulp tissue was investigated. A suitable porcine model for developing pulp cultures is useful for preliminary trials and for comparison to human pulp tissue.

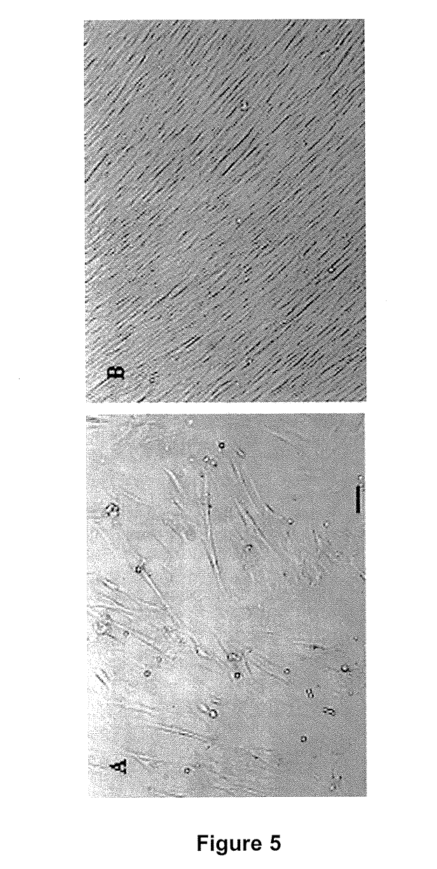

The methods were used for explanting human pulp tissue and culturing such tissue for outgrowth and subsequent culturing of cells. Porcine pulp fibroblasts were observed to grow faster than human pulp cells and their attachment morphology was slightly different from those of human origin. Initial and continued explant attachment to the culture flask surface was critical for outgrowth to occur. Contrary to other soft tissues, pulp explants that floated in culture medium did not produce cells. Explant pieces that became dislodged were removed from the flask and reattached to the bottom of another flask. Such reattached explants were able to produce cells. It was observed that outgrowth from porcine explants began as early as 3 days and as late as 12 days. Colonies that were visible by eye grew around the explants. Over time, colonies also formed at non-explant sites. The cell morphology varied with culture duration from round, to stellar, to elongated (FIG. 5A and FIG. 5B). Elongated cells eventually formed an oriented structure. After significant outgrowth occurred, i.e., a few hundred cells, the time for the culture to reach confluency in a T-75 flask varied from three to six weeks. Time from tooth pulp extraction to obtaining a flask of confluent cells ranged from about one to two months. Trypsinizing the explant outgrowth cells yielded approximately 1×10⁷ cells per flask (SD=9×10⁶).

### Example 2

### Collagen and Alginate Gel Fabrication and Cell Embedding

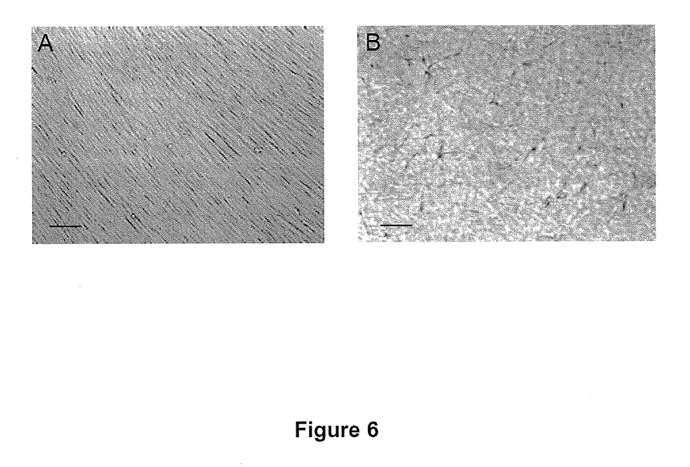

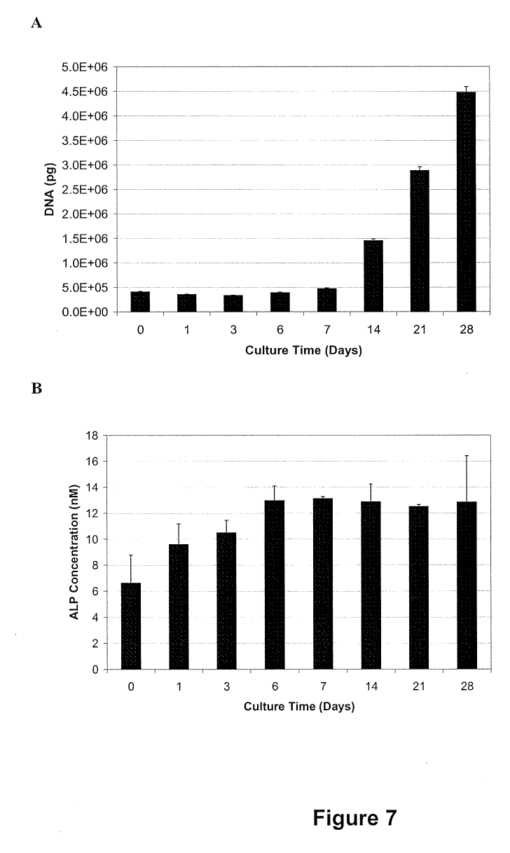

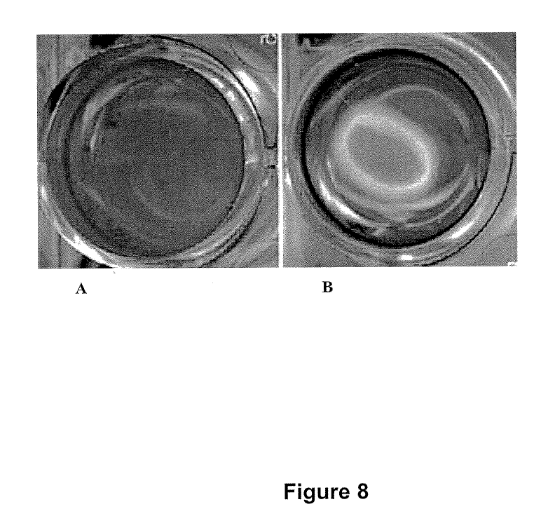

Human pulp cells were embedded and grown in type I collagen gel for 6 weeks as described herein. To prepare a matrix containing pulp cells and collagen, 1.5 ml of cell suspension (3.1×10⁶ cells/ml) in DMEM was mixed on ice with 5.0 ml of 3.1 mg/ml collagen solution (Vitrogen, Palo Alto, Calif.), 0.5 ml HEPES (25 mM) and 0.5 ml DMEM to a final concentration of 2.0 mg/ml of collagen in the collagen-cell solution. Collagen-cell solution was poured into the wells of a 6 well culture plate. The plate was incubated for 2 hours at 37° C. After polymerization, the culture media (Dulbecco's Modified Eagle's Medium (DMEM) supplemented with 10% fetal bovine serum (FBS), 100 UI/mL penicillin and 100 μg streptomycin and 5% non-essential amino acids) and the media was changed every two days. After 14 days these unrestrained gels containing cells exhibited noticeable contraction and became detached from the well surfaces. After 40 days, the free-floating collagen gel containing cells contracted 40% in diameter. Controls without cells exhibited no noticeable contraction. In addition to gel contraction; cell viability, growth and alkaline phosphatase (ALP) activity were determined. Live-dead staining revealed that the pulp cells were viable and homogenously distributed throughout the collagen gel (FIG. 6B). Cell morphology in collagen gel changed with time from an initial rounded shape to an elongated shape similar to cells grown on the culture flasks. These cells later became elongated, and they proliferated in collagen (FIG. 7A). Cell ALP activity increased for the first week and stabilized thereafter (FIG. 7B). Extensive gel contraction was observed and it coincided with significant increase in cell number, reaching 53.98±21.30% by day 28 (FIG. 8). Overall, these results demonstrate that type I collagen gel supported pulp cell growth and ALP activity, although the gel contracted significantly in vitro.

Human pulp fibroblasts were also embedded and grown in alginate gel. Cells (2.8×10⁶ cells/ml) were mixed with either a 1.0% wt/vol or a 3.0% wt/vol alginate solution (Sigma). The cell-alginate solution was dropped into a stirred 100 mM CaCl₂ solution through a 21 gauge needle. Beads were removed from solution after 15 minutes and washed in PBS. The cell embedded in alginate was placed in the culture media solution and stored at 37° C. incubator with 5% CO₂. Culture medium was changed every two days.

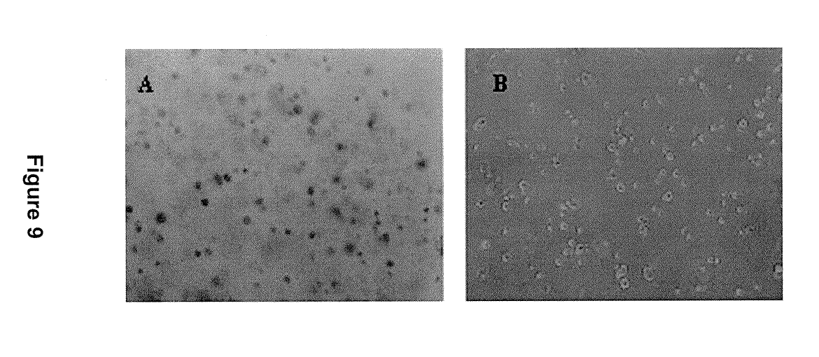

It was observed that the cells remained viable in the alginate gel and were homogenously distributed through out the gel (FIG. 9A). In contrast to collagen gels (FIG. 9B), there was no change in cellular morphology in alginate with time.

### Example 3

### Mechanical Properties of Porcine Pulp Tissue

An unconfined compression testing apparatus (of a type that can be used for cartilage explant testing) was used (Mauck et al, 2000) to test hydrogel mechanical properties. Porcine tissue was used as a testing tissue as it is relatively readily available compared to human pulp tissue. For the testing, the sample was cut into a 4×4×2 mm section, with the 2 mm axis in the buccal-lingual direction. A tare load of 2 g was applied. With a ramp speed of 1 μm/s, deformations of 10% and 20% were applied. For each deformation stress-relaxation curves (force vs time) were recorded utilizing a 50 g load cell. The equilibrium modulus was determined for each applied deformation. The measured modulus was calculated to be 1.3 kPa. Previous estimates of pulp properties in the dental finite element literature include 0 Mpa (megapascals) (Thresher, 1973), 0.003 (Toparli et al, 1999), and 3 Mpa (Williams et al, 1984). An increased sensitivity can be gained with a 10 g load cell instead of the 50 g load cell utilized as described herein. With a 10 g load cell, a lower smaller tare load and smaller specimen size can be used. The disclosed subject matter comprises established primary human pulp fibroblast cultures and methods for culturing; methods for embedding pulp cells in both type I collagen and alginate hydrogel matrices; and methods for determining the mechanical properties of human pulp.

### Example 4

### Establishment of Human Pulp Cell Cultures from Pulp Explants

To obtain cells suitable for testing matrices and constructs, and for transplantation using such matrices and constructs, pulp cell cultures are established. In one example of such culture preparation, non-carious premolars and third molars from healthy individuals were collected, e.g., from surgical waste. Tooth surfaces were washed with 70% ethanol. Teeth were cracked to extract the pulp tissue. Pulp was removed from the cracked tooth under sterile conditions.

The pulp is washed five times in DMEM supplemented with 2% penicillin (10,000 IU)-streptomycin (10 mg/ml) solution, and 5.0 mg/ml of amphitericin B (Sigma, St. Louis, Mo.). The pulp was cut into pieces with a sterile surgical blade. Pulp pieces generally have sides approximately 2 mm in length. Pulp pieces were removed from the petri dish and placed inside a T-75 flask with only a few drops of wash solution. To insure that the pulp pieces attach to the bottom of the flask, the flask was then placed in an incubator for 45 minutes. If pieces do not attach, some wash solution can be removed or the solution spread by rocking the flask and placing it back in the incubator for another 15 minutes. After attachment a minimal amount of “explant medium” (wash medium with 10% FBS) was added to the flasks. The flasks were then incubated for about 10 days.

After the incubation period, the flasks were gently removed from the incubator. If there was sufficient outgrowth (approximately 50 cells), explant medium was added to bring the medium volume to approximately 13 ml. During this time if any explant pieces were floating they can be removed, for example, using a sterile pipette, and placed into a T-25 flask for reattachment. Once colonies formed around the explants (a few hundred cells) the medium was changed from the above explant medium to modified explant medium containing wash medium containing FBS, 1% pen/strep and antifungal agent. As discussed above, this generally occurred by about day 14. The medium was then changed every other day with 10 ml of medium. Time to reach confluency depended on the number of attached pulp explants in the T-75 flask, but generally occurred about six weeks after tooth extraction.

This is a general protocol for preparation of pulp cells suitable for culture and seeding a matrix to generate constructs for use in restoration of a damaged tooth.

### Example 5

### Cell Seeding and Response to Three Dimensional Constructs

To identify a matrix that can support growth of pulp cells, pulp cells are seeded into a test matrix, cultured, and various properties of the cells are assayed. In one example, cells that have grown out of pulp tissue implants are harvested and are seeded in alginate: the cells are added to a syringe alone with the alginate solution (see fabrication section, supra). The cells become embedded in the alginate after the cell/alginate solution drops into a CaCl₂ solution.

To embed cells in collagen, the cell solution is added to the collagen solution after neutralization but before polymerization. The resulting cell/collagen mixture is then incubated at 37° C. for 2 hours.

Cellular attachment and growth morphology are examined using histological staining and scanning electron microscopy (SEM). At selected time points, the samples are washed three times with PBS to remove unadhered cells.

To examine morphology of the cells in a test matrix, histochemical staining is performed. For example, a cell/matrix sample is fixed in 4% paraformaldehyde, dehydrated in ethanol, and embedded in paraffin using methods known in the art. The sample is then sectioned and stained with hematoxyline and eosin using methods known in the art.

For SEM analysis, a sample is first dehydrated using an ethanol drying series, and dried in Freon overnight in a chemical hood. Prior to imaging, the sample is coated with carbon to eliminate charging effects.

Cell proliferation is determined using the Picogreen™ dsDNA Quantitation assay (Molecular Probes, Carlsbad, Calif.) where fluorescence intensity is correlated to DNA concentration.

To ascertain cell phenotype and the suitability of a test matrix for promoting and maintaining differentiation of pulp cells, marker proteins or RNAs for marker proteins and mineralization within the matrix are assayed. For example, alkaline phosphatase (ALP) synthesis, osteocalcin production, and the formation of a mineralized matrix by these cells can be determined.

ALP expression is quantified using a colorimetric assay. The samples are incubated at 37° C. for 30 minutes in 0.1 M Na₂CO₃ buffer containing 2 mM MgCl₂ with disodium p-nitrophenyl phosphate (pNP—PO4) as the substrate. Standard solutions are prepared by serial dilutions of 0.5 mM p-nitrophenol (pNP) in Na₂CO₃ buffer. Enzymatic activity is then expressed as total nmoles of pNP produced per min per total cell number. Absorbance is measured at 415 nm by a Spectrofluor reader (Tecan, Research Triangle Park, N.C.). Induction of ALP synthesis indicates that the test matrix can support differentiation and maintenance of pulp cells.

The synthesis of osteocalcin can be determined using an immunoassay (e.g., NovoCalcin™, Metra Biosystems, Inc., Mountain View, Calif.).

The formation of mineralized nodules is examined by SEM/EDXA, and the specific Ca/P ratio is calculated based on a hydroxyapatite standard. Mineralization can be further confirmed using Alizarin Red S (ALZ) staining specific for calcium. For ALZ staining, a sample is washed in double distilled H₂O, and incubated in 40 mM Alizarin red solution for 10 minutes. After additional washes, the matrix scaffolds are incubated in 10% cetyl pyridinium chloride for 15 minutes to solubilize reacted ALZ. In this assay, serial dilutions of 1 M CaCl₂ are used as standards. ALZ concentration per cell is calculated as molar equivalent CaCl₂ divided by the average cell number. Absorbance is measured at 570 nm using a Tecan Spectrofluor system (Tecan, Research Triangle Park, N.C.).

The expression of osteocalcin and dentin sialophosphoprotein (DSPP) is detected by reverse transcription followed by polymerase chain reaction (RT-PCR). For this purpose, cells are released from alginate by methods known in the art using sodium citrate, and from collagen gels using collagenase digestion. Cells can be released from chitosan using chitinase digestion. Total RNA is isolated using Rneasy® kit (Qiagen, Valencia, Calif.). First strand cDNA is synthesized using Superscript™ (Invitrogen, Carlsbad, Calif.). PCR is performed using the following primer sets: osteocalcin, sense 5′-CATGAGAGCCCTCACA-3′ (SEQ ID NO: 1) and antisense 5′-AGAGCGACACCCTAGAC-3′ (SEQ ID NO:2); DSPP, sense 5′-GGCAGTGACTCAAAAGGAGC-3′ (SEQ ID NO:3) and antisense 5′-TCATATTTGGCAGGTTTTTCT-3′ (SEQ ID NO:4). PCR is performed for 35 cycles at an annealing temperature of 56° C. PCR products are analyzed using 1.5% agarose gel electrophoresis and visualized by staining with ethidium bromide.

Expression of osteocalcin and DSSP indicate that the test matrix can promote expression proteins expressed in differentiated cells.

### Example 6

### Mechanical Properties of Pulp Tissue

The mechanical properties of pulp tissue are determined utilizing unconfined compression tests (Temenoff, 2002). Coronal pulp tissue is shaped into approximately 3 mm side cubes while frozen. The mechanical properties are measured along the long axis of the tooth as well as the two perpendicular axes. Spatial variations in mechanical properties are also investigated. When possible, multiple samples can be obtained from each tooth. The tooth number as well as the location within the pulp chamber is recorded. The equipment is the same as that used for the gels except that unconfined compression is applied. Due to expected tissue inhomogenity with anatomical location and possible anisotropy, a relatively large number of samples can be tested (e.g., at least about 20) per direction and/or anatomical location. These methods can be suitable for mammalian pulp cells, e.g., of porcine or human origin.

The mechanical properties of the pulp tissue provide a standard for desirable mechanical properties of a matrix or matrix-containing cells that are suitable for use in constructs for transplantation to a damaged tooth, i.e., to promote repair of a damaged tooth.

The protocol for obtaining pulp cells from explant outgrowth has been established. Initial and continued explant attachment to the culture flask surface is important for outgrowth to occur. Explants that floated in culture media did not produce cells. Explant pieces that became dislodged were removed from the flask and reattached to the bottom of another flask. Outgrowth began as early as 3 days and as late as 12 days. Colonies visible by eye grew around the explants. With time, colonies also formed at non-explant sites. The cell morphology varied with culture duration from round, to stellar, to elongated. Elongated cells eventually formed an oriented structure. After significant outgrowth occurred, i.e., a few hundred cells, the time for confluency in a T-75 flask varied from three to six weeks. Time from tooth extraction to obtaining a flask of confluent cells ranged from one to two months. Trypsinizing the explant outgrowth cells yielded 1×10⁷ cells per flask (SD=9×10⁶).

Outgrowth of porcine pulp tissue was initially investigated. This established parameters for protocols. After the protocol was established with porcine samples, human teeth were utilized. Porcine cells grew faster and cell morphology was slightly different than human cells. Cultured cells can be harvested and frozen using methods known in the art, generally after the first or second passage in culture.

### Example 7

### Gel Fabrication and Cell Embedding

Human pulp cells were embedded in alginate gel. Cells (2.8×10⁶ cells/ml) were mixed with either a 1% or 3% wt/vol % alginate solution (Sigma, St. Louis, Mo.). The cell alginate solution was dropped into a stirred 100 mM CaCl₂ solution through a 21 gauge needle. Beads were removed from solution after 15 minutes and washed in PBS. The cell embedded alginate was placed in the culture media solution and stored at 37° C. incubator with 5% CO₂. Medium was changed every two days.

Human pulp cells were embedded in collagen I gel using 1.5 ml of cell suspension (3.1×10⁶ cells/ml) in DMEM mixed on ice with 5.0 ml of 3.1 mg/ml collagen solution (Vitrogen, Cohesion Technologies, Palo Alto, Calif.), 0.5 ml HEPES (25 mM) and 0.5 ml DMEM to make a final concentration of 2.0 mg/ml collagen/cell solution. Collagen/cell solution was poured into a square mold inside a petri dish. The dish was incubated for 2 hours and 37° C. After polymerization, culture medium was added. Medium was changed every two days.

### Example 8

### Pulp Fibroblast Culture in Collagen Gels

To demonstrate the culture of human pulp cells, non-carious premolar and third molar teeth were obtained. Pulp was extracted from the teeth and the tissue explants were cultured in DMEM supplemented with 10% FBS, non-essential amino acids (NEAA), and antibiotics (pen/strep). After culturing as described herein, cells that had grown out of the explants were harvested, transferred to a fresh flask, and cultured.

Cultured cells were then seeded into DMEM-HEPES-MEM-Type 1 collagen gel. Samples were prepared containing cells at 2×10⁵ cells/ml, a final collagen concentration of 2 mg/ml, and a pH of 9.0. To prepare the seeded gel (a gel matrix containing cells), the collagen and cell mixture was placed on ice and incubated for 2 hours at 37° C. at 5% CO₂ to allow polymerization to occur. After the polymerization step, Culture medium (0.5 ml) was added to each well overnight. An additional 1.0 ml of medium was added then added and medium was exchanged every other day for 28 days.

Various matrix conditions were tested to identify those that are suitable for culturing pulp cells and for transplantation. One matrix condition was an unconfined gel matrix. For this preparation, culture wells were pre-coated with 2% bovine serum albumin (BSA) and incubated for 1 hour at 37° C. to create a surface that prevents gel attachment. Gel formulations were then placed in the prepared wells and gelation carried out as described herein. A second matrix condition that was tested was a confined gel matrix. In this case, Thermoplast cover slips (Fisher) were placed in the culture wells in which the matrix was prepared. A 22 gauge needle was used to scratch the remaining well surfaces to create multiple sites for gel attachment. In a third matrix condition, the matrix was prepared as a partially confined gel. In this case, a gel matrix was prepared under confined conditions (described supra) and a slit was made through the center of the confined gel to create a free edge.

Test matrices prepared under the three different conditions were tested under various conditions as follows: Group A=unconfined collagen gel without cells, Group B=unconfined collagen gel with cells, Group C=partially confined collagen gel without cells, Group D=partially confined collagen gel with cells, Group E=confined collagen gel without cells, and Group F=confined collagen gel with cells. The test matrices were then incubated in medium at 37° C., 5% CO₂.

For all test matrices, minimal gel contraction was observed during the first two weeks of culture. After 14 days, gels in all groups contracted and began to detach from the well surfaces.

Confined gels contracted the least (18.97%), with 23.37% for the partially confined and 53.98% for the unconfined gels. Unconfined gels exhibited the highest rate of contraction at 3.86% versus 1.36% per day for the unconfined group.

Cell morphology was also examined in those matrices containing cells. In general, cells exhibited a spherical cellular morphology upon embedding in the collagen gel. By day 2, a small percentage of pulp cells began to elongate and develop orientation patterns in the loaded gels. Pulp cells grown in the unconfined gel were found to be randomly located in the gel. By day 28, the density and number of cells increased dramatically in comparison to that of day 0, suggesting that collagen matrix I is an appropriate matrix for pulp cellular proliferation and differentiation. Cells became elongated and oriented along the free edge for the partially confined gels. Cell proliferation in confined and partially confined gels entered the plateau phase of cell growth by day 21, while that of the unconfined gels continued to increase, entering the exponential phase. These findings indicate that pulp cells in unconfined gels are actively proliferating, while those in the confined or partially confined gels can be induced to differentiate instead of proliferating.

Confined and partially confined gels visibly exhibited an increase in cells and orientation compared to the unconfined gels, indicating that conditions for pulp fibroblast growth and differentiation are favored under some degree of restriction and loading of the collagen gel.

Human pulp cells proliferated in the collagen type I matrix over time, and responded to the local mechanical environment of the gel.

These findings demonstrate that collagen gel is useful as a matrix for pulp tissue engineering. They also illustrate the use of a method for identifying a composition that is useful for culturing pulp cells, e.g., for transplantation.

### Example 9

### Pulp Cell Cultures in Chitosan Hydrogel

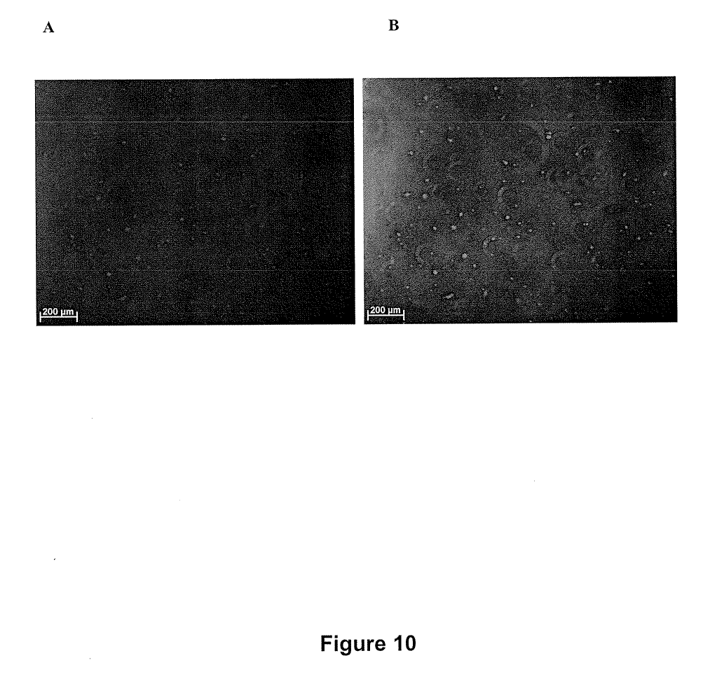

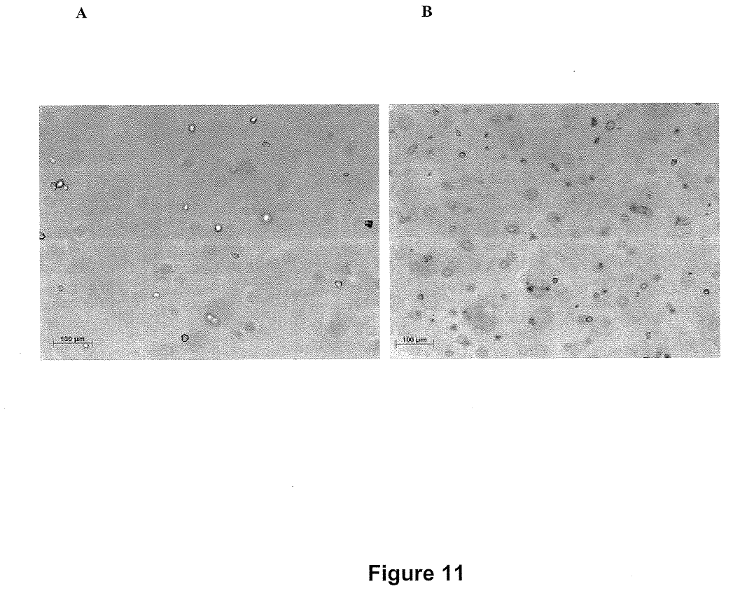

As described above, chitosan hydrogel is a material that can be used in compositions of the present disclosed subject matter. Experiments were conducted to demonstrate the suitability of chitosan for this application. Specifically, pulp cells prepared as described herein were embedded in a chitosan hydrogel film by mixing 1.36 mL of cell suspension (2.0×10⁵ cells/ml) with 8.0 ml of 2.5% chitosan solution (89.4% deacetylation, Spectrum Chemicals, Gardena, Calif.) resulting in a gel concentration of 3% and cell density of 2.0×105 cells/mL. Aliquots (1 mL) of the cell+chitosan solution was gelled in the culture well with glutaraldehyde (40 μL, Sigma). was pipetted into the cell culture well (12-well plate) to allow gelation. Cell viability and morphology within the chitosan hydrogel were examined over time. As shown in FIGS. 10A and 10B, pulp cells remained viable in the chitosan hydrogel. Cell number also increased over 6 days as indicated in FIGS. 11A and 11B. Pulp cells remained spherical in the gel and were evenly distributed through out the hydrogel. Cell culture can also result in the contraction of chitosan hydrogel, with gel thickness decreasing over 50% by day 3 and stabilizing afterwards. In vitro culture lead to minimal contraction of chitosan hydrogel, with 10% decrease in gel diameter by day 3 and stabilizing thereafter. These data demonstrate that chitosan hydrogel (chitosan) is useful for culturing pulp cells, e.g., culturing pulp cells to be used in, or as, constructs for pulp regeneration and tooth repair.

### Example 10

### Pulp Cell Culture in PEG-Fibrinogen Hydrogel

Pulp cell viability and differentiation was evaluated in polyethylene glycol (PEG)-fibrinogen hydrogel. Briefly, a precursor solution of the PEGylated-fibrinogen gel was synthesized following the methods of (Almany and Seliktar, 2005). To form the hydrogels, the precursor PEGylated fibrinogen solution was combined with a 0.1% (w/v) photoinitiator solution prepared by dissolving 10% w/v Irgacure®2959(CIBA) in 70% ethanol. To form the cell-laden hydrogel, the pulp cell suspension was first mixed with the precursor solution, resulting in a hydrogel concentration of 10% (w/v) with a final cell concentration of 1.6×106 cells/mL. Aliquots (100 μl) was then added to culture well and UV polymerized at 365 nm (4-5 mW/cm2, 5 min). Additional PEG-diacrylate (PEG-DA, Sigma) is added to the precursor solution in order to increase total PEG content to 2% and improve construct integrity. After crosslinking, the hydrogels were incubated in 1 mL of fully supplemented DMEM, and cell morphology was examined over time.

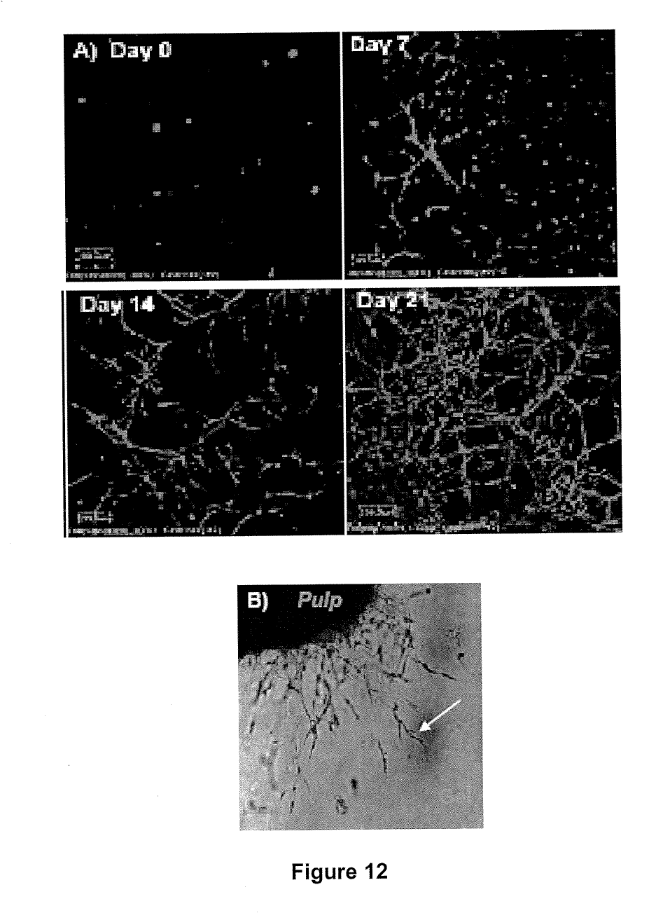

Immediately post seeding (FIG. 12A, day 0), similar to that of the chitosan gel, the pulp cells in PEG-fibrinogen were spherical. One day later, cellular extensions were visible and the cells assumed a stellate form by day 7. Three-dimensional cellular networks with extensive cellular contacts were apparent by day 21 (FIG. 12A) and the cells remained viable throughout the three-week culturing period. Gene expression analysis revealed that the PEG-fibrinogen hydrogel supported types I and III collagen expression at day 21, and type I collagen deposition was evident at day 14. These results demonstrate the feasibility of culturing pulp cells in PEG fibrinogen hydrogel is useful for culturing pulp cells, e.g., culturing pulp cells to be used in, or as, constructs for pulp regeneration and tooth repair.

### Example 11

### Pulp Cell Infiltration into Hydrogel Matrix

For pulp tissue engineering, the hydrogel can be used either as a scaffold for pulp repair after pulpotomy or for total pulp regeneration after pulpectomy. For pulp repair, the hydrogel can be applied after removal of the infected part of the pulp, and cell infiltration from the remaining pulp into the hydrogel can enable new pulp formation within the hydrogel. For pulp regeneration, infiltration of host stem cells and blood vessels from the apex of the tooth into the hydrogel can be important for graft stability and eventual integration with the host. Using PEGylated fibrinogen, outward cell migration from a pulp explant into the acellular hydrogel over two-weeks we examined. Specifically, dental pulp tissue obtained from an upper third molar was sectioned into 1×1 mm pieces using a sterile scalpel. The precursor PEG-fibrinogen solution was mixed with the photoinitiator solution (0.1 v/v %) as described above, and pipetted into the well containing the pulp explant. The mixture was photo-crosslinked at 365 nm with the explant fully immersed in the hydrogel. The explant+PEG-fibrinogen hydrogel were cultured in fully supplemented DMEM, and cell migration from the embedded pulp explant into the hydrogel matrix was monitored daily via phase contrast light microscopy. As seen in FIG. 12B, the first signs of cellular invasion into the PEG-fibrinogen hydrogels occurred on day 5, with the rate of cell outgrowth at approximately 100 μm per day. Initially, the migrated cells in the gel appeared polarized and oriented radially outward from the embedded pulp tissue. Over time, the cells began to orientate toward each other and form connections within the hydrogel (FIG. 12B). These results suggest that the hydrogel scaffold supports pulp cell infiltration and is useful for culturing pulp cells, e.g., culturing pulp cells to be used in, or as, constructs for pulp regeneration and tooth repair pulp capping and total pulp regeneration.

### Example 12

### Effects of Platelet-Rich Plasma Treatment on Pulp Cells

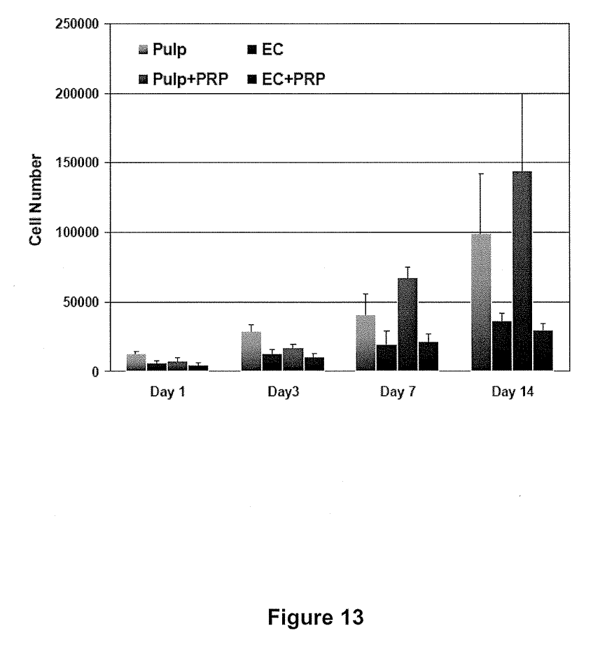

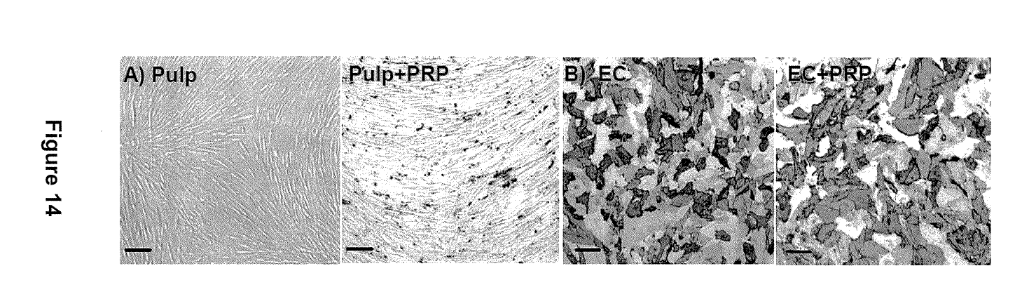

As PRP treatment of pulp cells have not been reported, pulp cell response to platelet-rich plasma (PRP) in 2-D culture, was evaluated. The PRP was prepared following the method of (Landesberg et al, 2000). Briefly, 60 ml of venous blood from healthy adult volunteers was first mixed with a solution of anticoagulant-citrate-dextrose. The mixture was then centrifuged at 200 g for 15 minutes (ACE Surgical Supply). The plasma and buffy coat layers were collected and spun at 200 g for another 10 minutes. After discarding the platelet-poor plasma, the lower half of the plasma and pellet was re-suspended and collected as the platelet-rich plasma or PRP. The average platelet count of this preparation method was determined to be 1.1×106 platelets/μl77. For the PRP treatment experiment, pulp cells isolated via enzymatic digestion (Nakashima et al, 1991; Gronthos et al, 2000) and human iliac artery endothelial cells (EC, purchased from ATCC) were cultured on wells pre-coated with gelatin (0.1%, Sigma). The pulp cells and EC were seeded at 1.7×106 cells/mL and 2.4×106 cells/mL, respectively. Both cultures were grown in endothelial cell maintenance medium, which was formulated based on Kaighn's modification of Ham's F-12K medium with 2.0 mM L-glutamine, supplemented with 1.5 g/L heparin and 0.03 mg/ml endothelial growth supplement (all purchased from ATCC). This media was further supplemented with 10% FBS. For the study, PRP was added to the endothelial cell maintenance medium at a concentration of 20 μl/mL, and the experimental cultures (pulp+PRP, EC+PRP) were treated every other day with 1 mL of this PRP-containing medium. Untreated monolayer cultures of pulp cells (Pulp) and endothelial cells (EC) served as controls. It was found that the pulp cells proliferated in the endothelial cell maintenance media and PRP treatment had minimal effect on cell growth over time (FIG. 13). However, positive staining for endothelial cell transmembrane protein CD31 or platelet-endothelium cell adhesion molecule (PECAM-1) was observed in pulp cells treated with PRP (FIG. 14A). No observable effects was seen in PRPtreated endothelial cells (FIG. 14A). Note that CD31 is strongly expressed in endothelial cells and weakly expressed in platelets, thus further investigation is needed regarding the angiogenic potential of PRP. Overall, these results suggest that PRP can induce angiogenesis in pulp cells is useful for culturing pulp cells, e.g., culturing pulp cells to be used in, or as, constructs for pulp regeneration and tooth repair.

### Example 10

### In Vitro Co-Culture of Pulp Cells

A co-culture model for culturing pulp cells was developed and tested. Pulp cells embedded in chitosan hydrogel beads (cell plus chitosan) as described herein were co-cultured with a preformed monolayer of pulp cells (0.1×10⁶ cells/cm²) in a 24-well plate. The cell plus chitosan beads were formed by mixing 200 ml of cell suspension with 2.0 ml of 2.5% chitosan solution and dispensing the solution drop-wise into 22.4% w/v sodium sulfate (LabChem. Inc.) solution using a 26½ gauge needle. The final cell concentration in the chitosan hydrogel was 2.0×10⁴ cells/ml. The cell plus chitosan beads were then cultured on top of a monolayer of pulp cells over time.

This co-culture model is designed to simulate the intended clinical application, where after the removal of infected pulp, a cell plus chitosan hydrogel is placed directly in the pulp chamber in direct contact with the underlying pulp tissue. The model is based on developing a context in which the interaction between the cells from the underlying pulp and the cells embedded in the hydrogel can modulate the overall cell response within the hydrogel. Such methods can be used to test the ability of different cell types, e.g., stem cells, and/or cells prepared under various conditions, for their ability to demonstrate features such as expression of differentiated proteins, cell proliferation, and cell morphology, that are suitable for use in a composition of the disclosed subject matter.

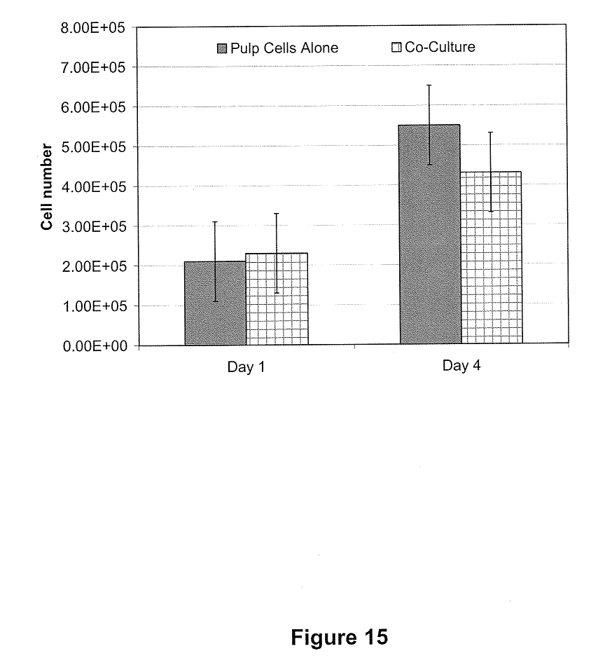

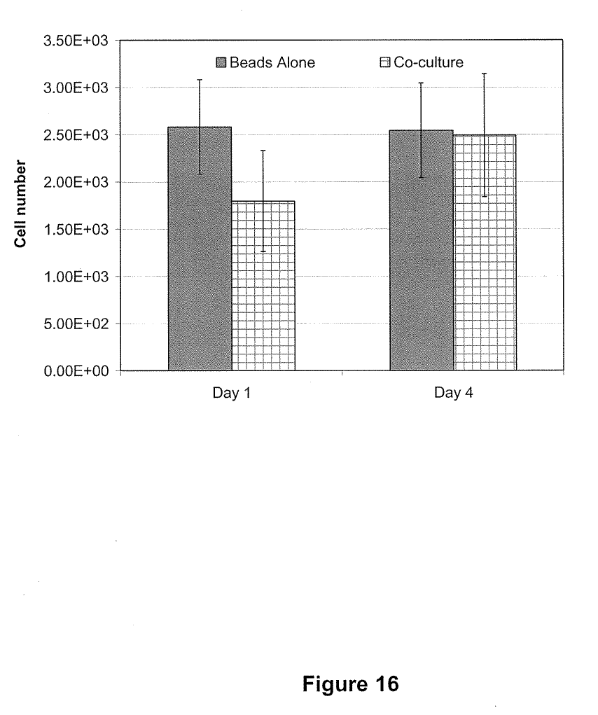

Cell growth for both the pulp monolayer and the pulp cells embedded in chitosan beads were compared as a function of co-culture (FIG. 15 and FIG. 16, respectively). In both cases, co-culture had little effect on cell growth over the short time period examined. Cell number continued to increase in monolayer culture, and cell number remained relatively constant in the chitosan hydrogel beads, suggesting differences between two-dimensional and three-dimensional cultures.

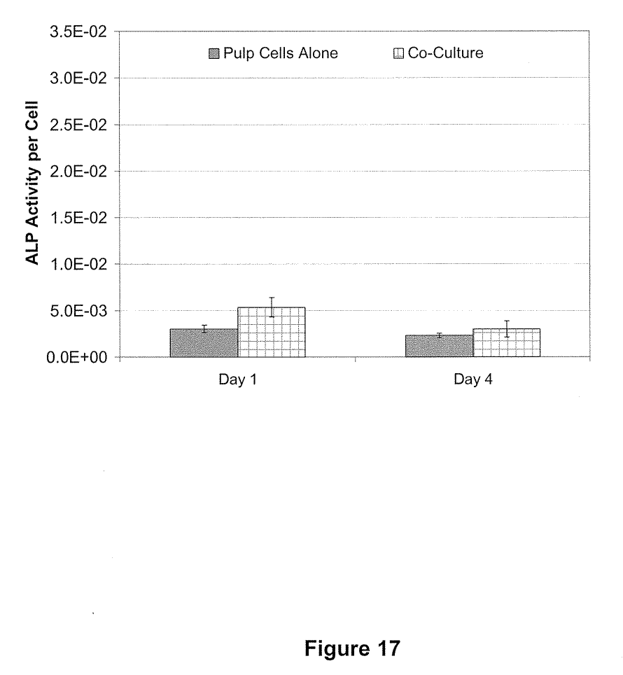

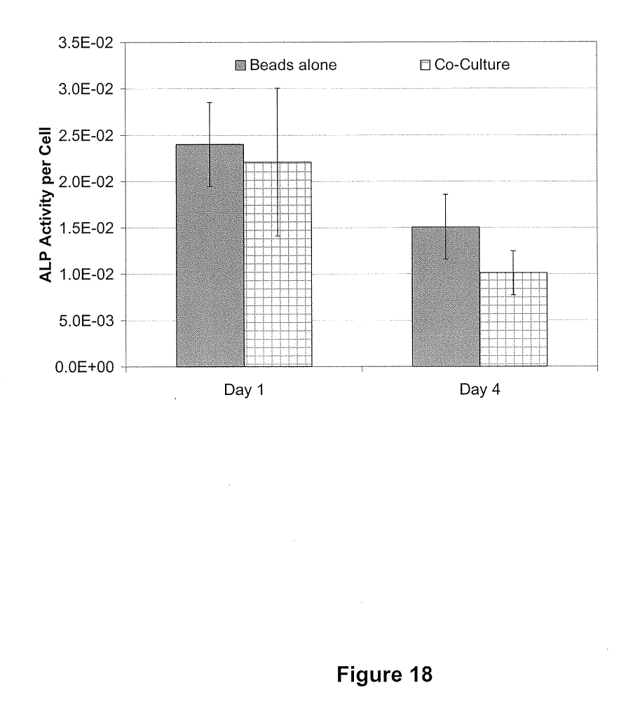

Co-culture increased the ALP activity of monolayer pulp cells (FIG. 17) but had no significant effect on pulp cells grown in three-dimensional chitosan beads (FIG. 18). ALP activity per cell was significantly higher in three-dimensional cultures compared to the monolayer, further confirming the importance of three-dimensional culture in determining cell response.

These data also demonstrate that co-culture is a useful method for culturing pulp cells in two-dimensional cultures or in three-dimensional cultures.

### Example 11

### Culture of Pulp Cells on Nanofiber Mesh

To demonstrate the ability of a nanofiber mesh (scaffold) to support growth of pulp cells, an electrospun mesh was prepared and used as a matrix for pulp cell culture. Briefly, an electrospun mesh was prepared using PLGA 85:15, IV=0.66-0.80 dL/g (Alkermes, Cambridge, Mass.); N,N-dimethylformamide (DMF) (surface tension (σ=mN/M; Fisher Scientific), and ethanol (σ=22.1 mN/m). Electrospinning parameters were 11 kV, needle tip/collecting plate distance of 90 mm, and a flow rate (qᵥ) of 1 ml/hour (syringe pump controlled). Meshes were cut into squares 1.5 cm×1.5 cm and sterilized using uv irradiation. Human pulp fibroblasts were harvested from culture and seeded directly onto the meshes (scaffolds) and were cultured in DMEM containing 10% FBS, 1% pen/strep, and 1% NEAA. The initial seeding densities were determined using fluorometric DNA detection methods (PicoGreen®). Cultures were examined at 1 day, 7 days, and 14 days after the initiation of cultures. Cultures were examined using SEM, assayed for ALP activity (fluorometric assay); DNA quantified using PicoGreen, and statistical analyses were performed using ANOVA and Tukey-Kramer.

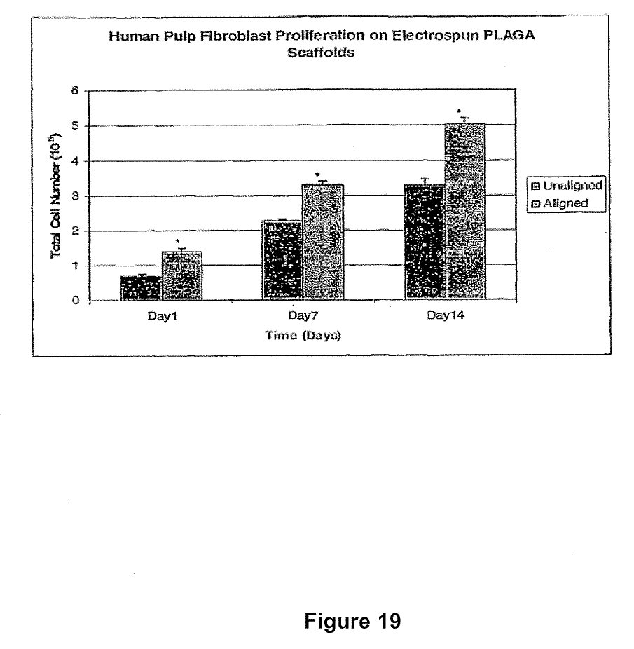

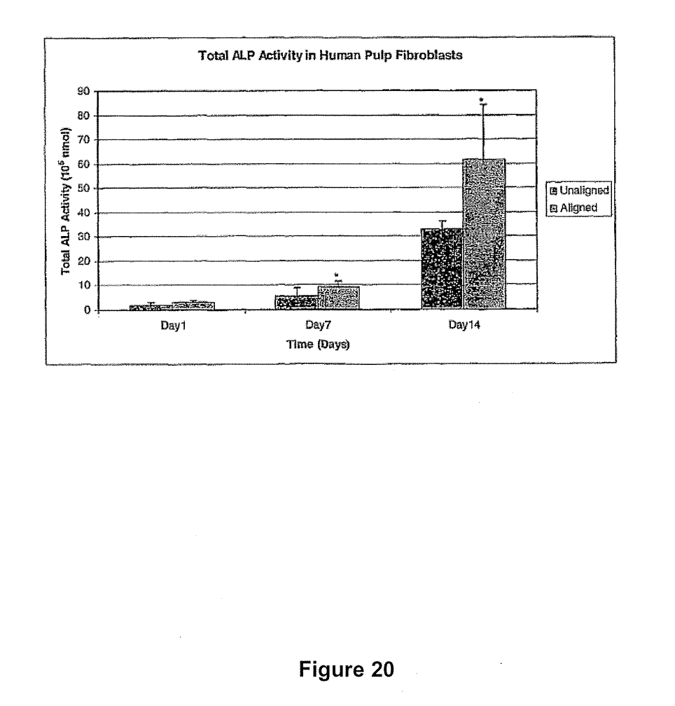

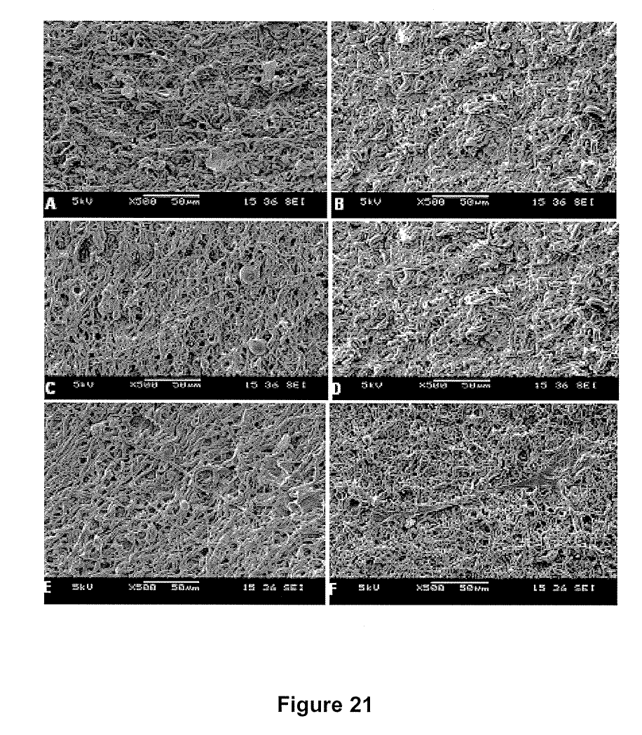

It was found that smooth (non-beaded) fibers were formed when a solution of 60% DMF and 10% ethanol was used to generate the fibers. Cells proliferated on the scaffolds (FIG. 19) and cell ALP activity increased over time in culture (FIG. 20). In addition, by day 14, cells had elaborated abundant cell matrix on the mesh (FIG. 21). Both cell proliferation and ALP activity were increased in those cultures grown on aligned mesh compared to those grown on unaligned mesh.

These data demonstrate that electrospun mesh can be used to culture pulp cells, and that cells cultured on such a matrix can proliferate and express proteins indicative of cell differentiation. These data also demonstrate a method of testing matrices for their ability to support pulp cell growth and differentiation.

### Example 12

### Mechanical Properties

Appropriate methods for determining pulp mechanical properties are useful for, e.g., identifying desirable parameters for constructs useful for transplantation. An unconfined compression testing apparatus was utilized (Ranly et al, 2000). One porcine pulp tissue sample was cut (4 mm×4 mm×2 mm) with the 2 mm axis in the buccal-lingual direction. A tare load of 2 g was applied. With a ramp speed of 1 μm/s, deformations of 10% and 20% were applied. For each deformation, stress-relaxation curves (force versus time) were recorded utilizing a 50 g load cell). The equilibrium modulus was determined for each applied deformation. The measured modulus was 1.3 kPa. The significance of this experiment is that the mechanical testing equipment used is appropriate, but a 10 g load cell can increase experimental sensitivity. With a 10 g load cell a lower smaller tare loads and specimen sizes can be used.

### Example 13

### Scaffold System

In order to produce an optimal scaffold design that supports cell growth and differentiation, pulp cell response to three candidate scaffolds for pulp tissue engineering can be compared. Angiogenesis within the optimized hydrogel scaffold can be stimulated by treatment with platelet-rich plasma (PRP).

A hydrogel-based scaffold is more suitable for pulp tissue engineering due to the inherently high water content found in soft tissues such as the pulp (e.g. 50-80%88). Pulp cell response in three different hydrogel scaffolds: chitosan, collagen type I, and polyethylene glycol (PEG)-fibrinogen can be compared. The three types of hydrogel have been successfully utilized in tissue engineering applications. Chitosan is a degradable biopolymer derived from the exoskeleton of crustaceans. In addition to its documented biocompatibility, it is considered for pulp tissue engineering due to its anti-bacterial potential. Bacterial infection compromises pulp vitality and is the primary reason for performing RCT. An anti-bacterial matrix such as chitosan can be especially useful during the early stages of pulp healing. Collagen (type I) is the primary component of pulp tissue, and while it does not offer protection against infection and is less cost-effective, it supports pulp cell growth and differentiation. The third hydrogel is the PEG-fibrinogen hydrogel comprised of a fibrinogen-based matrix crosslinked with PEG-diacrylate or PEG-DA. Additional PEG-DA can be added to the gel in order to customize the construct degradation and mechanical properties (Dikovsky et al, 2006). The PEGylated fibrinogen gel is useful for tissue engineering since unlike many polymers which degrade via hydrolysis, its degradation is cell-mediated which in turn allows greater control in matching cell biosynthesis to matrix degradation. Moreover, the in situ photopolymerization of the PEG-fibrinogen gel renders it relatively easy to implement during surgical procedures. As culturing of pulp cell in these three hydrogels has not yet been reported, cell growth and differentiation in these matrices, as well as the effect of in vitro culture on hydrogel structural and mechanical properties can be evaluated.

The experimental groups can be cells seeded on each type of hydrogel matrix, while the control groups include acellular gels and a monolayer of pulp cells. The three variables to be tested include hydrogel concentration, cell seeding density and culturing duration. Specifically, the gel concentrations to be tested for each type of hydrogel are: chitosan (1.0, 2.5, 5.0 w/v %), collagen I (1.0, 2.0, 4.0 mg/ml), and PEG-fibrinogen (1, 2, 4% additional PEG content). The gel concentrations are selected based on results from preliminary studies. Optimizing cell seeding density is also important for pulp tissue engineering, and the values (0.5×10⁶, 1.0×10⁶, 2.0×10⁶ cells/ml) are chosen based on reported and preliminary results in monolayer and hydrogel pulp cultures. Specifically, cell response and hydrogel matrix properties can be evaluated in vitro over a four-week culturing period (1, 3, 7, 14, 28 days). The cell-related outcome measures are cell morphology (n=3, by electron microscopy and histology) and proliferation (n=6, by total DNA assay (Lu et al, 2003), matrix synthesis exemplified in total collagen production (n=6, by hydroxyproline assay (Lu et al, 2005), total glycosaminoglycan (GAG, n=6, by Blyscan GAG assay (Lu et al, 2005), alkaline phosphatase (ALP) activity (n=6, by colorimetric assay (Lu et al, 2003), and mineralization (n=6, by quantitative Alizarin Red assay (Lu et al, 2003b). In addition, matrix distribution within the hydrogel can be assessed histologically via Immunohistochemistry (Lu et al, 2005; Lu et al, 2003b) (collagen types I and III, dentin sialoprotein [DSP] and osteopontin, n=3 each). Gene expression (housekeeping gene-GAPDH, DSP, and osteocalcin, n=6 each) can be determined by reverse transcriptase polymerase chain reaction (RTPCR) (Lu et al, 2003). The scaffold-related outcome measures can include gel geometry 13 (diameter/height, n=6), wet and dry weight (n=6), as well as mechanical properties (Teng et al, 2005; Salani et al, 2000) (n=6) as a function of gel concentration, cell seeding density and in vitro culture. Changes in gel geometry or gel contraction can be determined via quantitative image analysis methods (Teng et al, 2005). Since pulp mechanical properties have not been reported, gel mechanical properties (compressive modulus and yield strength) under applied compression can be determined. These values can be compared to that of the as-isolated native pulp tissue tested under similar conditions.

For cell response, the three hydrogels can support cell growth and viability over time due to their documented biocompatibility. Differences in cell morphology can exist between gel types, with the more physiologic morphology (elongated, spindle shaped) expected in the PEG-fibrinogen and collagen gels than in the chitosan gel (spherical). The exhibited cell morphology can also depend on the initial gel concentration. Extracellular matrix production by pulp cells in chitosan, PEG-fibrinogen and collagen matrices can be maintained, but the magnitude of response is likely to differ between gel types. With increasing cell seeding density, both cell number and biosynthesis can increase over time in these hydrogels. In addition, cell proliferation and differentiation can vary as a function of cell seeding density and culturing time. For scaffold properties, matrix contraction can be highest in the collagen gel when compared to PEG-fibrinogen or chitosan, and gel mechanical properties can increase over time with increasing gel concentration and cell seeding density. At each specific cell density or gel concentration, that gel matrix degradation can be balanced by cell matrix production, and the relationship between gel degradation and matrix production can be reflected in temporal changes in gel mechanical properties.

### Example 14

### Scaffold for Pulp Tissue Engineering

For each gel type, an optimal gel concentration and cell seeding density can be identified based on characteristic cell morphology, appropriate biosynthesis, expression of relevant phenotypic markers, as well as mechanical properties comparable to the native pulp. In addition, the scaffold exhibits minimal contraction or changes in geometry due to in vitro culture, and cell biosynthesis should be balanced with any gel degradation reflected in the loss of mechanical properties over time. While it is possible that no single hydrogel can meet all the requirements for the ideal pulp scaffold, the optimal hydrogel scaffold can be selected from these three candidate materials based on comparisons between gel types of at the same cell density or time point.

### Example 15

### Induction of Vascularization in Hydrogel Matrix Via PRP Stimulation

The hydrogel scaffold for pulp tissue engineering can be optimized to promote angiogenesis. The dental pulp is a highly vascularized tissue, with over 15% by volume of the peripheral pulp and as high as 40% within the center of the growing pulp comprised of blood vessels (Kishi et al, 1995). The formation of a vascularized matrix is a critical design parameter for pulp tissue engineering. In addition to being biomimetic, vessel formation and neovascularization can enable nutrient transport and waste removal from the pulp scaffold, and has the potential to expedite pulp healing and regeneration by encouraging host-mediated repair. The primary method for induction of vascularization in the hydrogel can be stimulation of pulp cell mediated angiogenesis with platelet-rich plasma (PRP). Platelets in the PRP provide an autologous source of angiogenic factors such as vascular endothelial growth factor (VEGF), basic fibroblast growth factor (bFGF) and platelet-derived growth factor (PDGF), which all have been shown to be upregulated by pulp cells upon injury. In addition, these factors present in PRP have been reported to promote angiogenesis and healing in the injured pulp (Derringer and Linden, 2004; Derringer and Linden, 2003; Grando et al, 2007) as well as other soft tissues (Tran-Hung et al, 2006; Anitua et al, 2005; Murray et al, 2007). PRP can be readily combined with a hydrogel matrix to promote vessel infiltration into the scaffold and encourage host-mediated repair. Preliminary results reveal that PRP treatment of monolayer cultures of pulp fibroblasts promotes the production of PECAM-1, suggesting that PRP treatment can induce angiogenesis. Tran-Hung et al. reported that paracrine interactions between pulp fibroblasts and endothelial cells promoted the endothelial cells to form tubular structures resembling capillaries in vivo (Tran-Hung et al, 2006). Our isolation method (i.e. collagenous digestion) can yield a mixed population of cells with angiogenic precursors are present in the pulp digest (d'Aquino et al, 2007), and they can contribute to the positive staining of PECAM-1 by PRP-stimulation. The concentration and method of PRP delivery optimal for angiogenesis within the pulp graft are not known. Therefore, two studies can be conducted to show that angiogenic factors in PRP can induce vascularization in 3-D hydrogel culture of pulp cells. The first study can focus on the effects of PRP delivery while the second study can determine whether increasing PRP concentration can enhance angiogenesis within the pulp cell-laden hydrogel.

### Example 16

### Effects of PRP Delivery on Pulp Cell Response

Pulp cell response to PRP delivered by direct addition to the culture medium or by premixing with the hydrogel matrix can be compared since PRP preparation methods significantly modulate the bioavailability of PRP-derived growth factors 10,77. The variable to be tested include the PRP delivery method and culture duration. The experimental group can be PRP (20 μl) added to the culture media (PRP-media), or PRP mixed with the cell laden hydrogel (PRP-gel). The volume of PRP can be adjusted such that its final concentration can be identical in both groups. The control groups can include untreated 2-D and 3-D cultures of pulp cells and endothelial cells, as well as acellular scaffold and endothelial cells stimulated with PRP. The volume of PRP used can be 20 μl. Pulp cell response can be evaluated in vitro over a four-week period (1, 3, 7, 14, 28 days). In addition to the cell-related outcome measures described above, angiogenesis can be characterized by the formation of tubular structures as well as positive staining for endothelial membrane protein CD-31 and von Willebrand's factor (vWF) in order to identify vessels lined with endothelial cells. Endothelial cells, upon stimulation by angiogenic factors, will organize into tubular structures. The formation of tubular structures in co-culture can be quantified using digital image analysis, by measuring the perimeter, area, or number of branches observed in the network (Teng et al, 2005; Salani et al, 2000). In addition, the production of VEGF, PDGF and bFGF by pulp cells can be determined via immunohistochemistry, and the concentration of these angiogenic factors in the cell culture media can also be measured by ELISA. The scaffold-related outcome measures described above can also be tested. PRP delivery via the hydrogel can promote angiogenesis by providing a local source of angiogenic factors. The optimal PRP delivery method identified can be applied to determine the effects of PRP concentration on pulp cell response.

### Example 17

### Effects of PRP Concentration on Pulp Cell Response

To determine the optimal concentration of PRP for angiogenesis within the hydrogel, PRP-treated cultures of pulp cells or endothelial cells grown in the hydrogel can be compared non-treated cultures of pulp cells or endothelial cells, in the hydrogel scaffold. Collagenous digestion can yield a mixed population of cells, thus the angiogenic cells can also be present in the single cultures of pulp cells. The volume of PRP (20, 40, 60 μl PRP/ml of media), as well as culture duration can be examined. PRP volume as the total amount of growth factors can be varied and can increase linearly with higher PRP volume. The samples can be evaluated over a four-week period (1, 3, 7, 14, 28 days). The cell-related and scaffold-related outcome measures described above, as well as the angiogenesis indicators can be determined. PRP treatment can promote angiogenesis by pulp cells, and the number of tubular structures can increase with increasing PRP volume. An optimal PRP volume for pulp graft vascularization can be identified.

### Example 18

### Isolation and Culture of Human Pulp Cells and Endothelial Cells

Non-carious premolars and third molars from healthy individuals can be collected from surgical waste. Tooth surfaces can be washed with 70% ethanol and the pulp can be extracted after cracking the teeth. The pulp can be washed five times in Dulbecco's Modified Eagles Medium (DMEM), supplemented with 2% penicillin (10,000 IU)-streptomycin (10 mg/ml) solution, and 5.0 μg/ml of Amphotericin B (Sigma). The pulp can be minced using sterile scalpels and digested using collagenase type II (0.05%, Worthington, Lakewood, N.J.). Cell suspension from the second digestion can be collected and cultured in Dulbecco's Modified Eagle's Medium (DMEM, Mediatech, Herndon, Va.), supplemented with 10% Fetal Bovine Serum (FBS, Atlanta Biologicals, Lawrenceville, Ga.), 100 UI/mL penicillin and 100 μg streptomycin (P/S, Mediatech, Herndon, Va.), and 5% non-essential amino acid (NEAA, Mediatech, Herndon, Va.). The cultures can be incubated at 37° C. in a 5% CO2 atmosphere, and the pulp cells can be expanded and only cells below passage four (4) can be used in the study. Human Iliac Artery Endothelial Cell Line (HIAEC) can be purchased from American Type Culture Collection (ATCC, Manassas, Va.). The cells can be expanded in culture in plates pre-coated with 0.1% gelatin. The endothelial cell maintenance media used can be based on Kaighn's modification of Ham's F-12K medium with 2.0 mM L-glutamine supplemented with 1.5 g/L heparin, 0.03 mg/ml endothelial growth supplement (all purchased from ATCC). The media is further supplemented with 10% FBS. Confluent cultures of HIAECs can be subcultured and maintained through maximum two or three passages at 37° C. in a 5% CO2 atmosphere.

### Example 19

### Cell Culture and Seeding in Hydrogel Constructs

Human pulp cells can be cultured in DMEM+10% FBS and supplemented with 2% penicillin (10,000 IU)-streptomycin (10 mg/ml) solution, and 5.0 μg/ml of Amphotericin B, at 37° C. and 5% CO2 until confluent. First to second passage cells can be embedded in the hydrogels and cell growth and differentiation on these substrates can be examined as a function of gel type, gel concentration, seeding density and culturing duration. The cells can be embedded in chitosan following the methods described above. To embed the cells in type I collagen, the cell suspension can be added to the bovine collagen solution after neutralization but before polymerization. Briefly, 1.5 ml of pulp cell suspension (3.1×106 cells/ml) in DMEM can be mixed with 5.0 ml of 3.1 mg/mL denatured collagen solution (Vitrogen), 0.5 ml HEPES (25 mM) and 0.5 mL DMEM to make a final concentration of 2.0 mg/mL of collagen/cell solution. The collagen/cell solution can then be poured into a mold, and incubated for 2 hours at 37° C. to allow polymerization to occur. For PEG-Diacrylate Synthesis, PEG-DA can be prepared from linear PEG-OH, (Fluka, Mw=10 kDa). In brief, acrylation of PEG-OH can be carried out under Argon by reacting a dichloromethane solution of PEG-OH (Aldrich) with acryloyl chloride (Merk) and triethylamine (Fluka) at a molar ratio of 1.5:1 relative to —OH groups. The final product can be precipitated in ice-cold diethyl ether and dried under vacuum for 48 hrs. Proton NMR (1HNMR) can be used to validate end-group conversion and to verify purity of the final product. The PEGylation of fibrinogen can be done according to Almany and Seliktar, 2005. Briefly, tris (2-carboxyethyl) phosphine hydrochloride (TCEP HCl) (Sigma) can be added to a 7 mg/ml solution of bovine fibrinogen (Sigma-Aldrich) in 150 mM PBS with 8M urea (molar ratio 1.5:1 TCEP to fibrinogen cysteines). Linear PEG-DA (10-kDa) can react for 3 hrs with the protein at a 4:1 molar ratio of PEG to fibrinogen cysteines. The PEGylated protein product is then precipitated in acetone and redissolved in PBS containing 8M urea at 7 mg/ml final fibrinogen concentration. The PEGylated protein products can be dialyzed against PBS at 4° C. for two days (Spectrum, 12-14-kDa MW cutoff), and characterized accordingly (Dikovsky et al, 2006). For hydrogel formation, the PEGylated fibrinogen precursor solution can be photopolymerized using 0.1% (w/v) Irgacure™2959 (Ciba Specialty Chemicals) and UV light (365 nm, 4-5 mW/cm2) exposure for 5 min. To form cell-laden hydrogels, pulp cell suspension can be mixed with the PEG-fibrinogen precursor solution, and all tissue culture experiments can be performed with 7-10 mg/ml PEGylated protein solution, and the solution can be supplemented with 10-kDa linear PEG-DA (1, 2, 4 mg/ml) in order to evaluate the effects of gel PEG content on cellular response. The cell-laden hydrogels can be incubated in 1 mL of fully supplemented DMEM, and cell morphology can be examined over time.

### Example 20

### Preparation and the Treatment of Pulp Cell Cultures with PRP

Platelet-Rich Plasma (PRP) can be prepared by a modification of the method of Landesberg et al. 2000. Briefly, 60 ml of venous blood from healthy adult volunteers can be mixed with Anticoagulant-Citrate-Dextrose (ACD). The ACD solution contained 13.2 g/L trisodium citrate, 4.8 g/L citric acid, and 14.7 g/L dextrose. The mixture can be centrifuged at 200 g for 15 minutes (ACE Surgical Supply Company, Inc; Brockton, Mass.). The plasma and buffy coat layers can be collected and spun at 200 g for 10 minutes. After discarding the platelet-poor plasma, the lower half of the plasma and pellet can be re-suspended and collected as the platelet-rich plasma. This preparation method usually yields an average platelet count of 0.5 to 1.0×106 platelets/μl (d'Aquino et al, 2007; Tsay et al, 2005).

### Example 21

### Characterization of Hydrogel Construct

### Morphology, Porosity, Contraction

For all gels, surface morphology, matrix organization, can be examined via environmental scanning electron microscopy (ESEM, FEI Company)92. Organization in the interior or cross section of the gels can be also be examined by ESEM. Matrix porosity can be determined via quantitative image analysis (n=5) of the SEM and light microscopy images using the Zeiss Axiovision modular image analysis package (Axiovision 3.1, Zeiss). Quantitative image analysis can be used to measure the contraction of the gels as a function of cell culture and time 13. Gel mechanical properties including shear and compressive moduli (n=6) can be determined under static compression and dynamic torsional shear using a shear-strain controlled rheometer (TA Instruments) following our published methods of Teng et al, 2005, Salani et al, 2000 and Zhu et al, 1993. Briefly, a 15% compression can be applied after a 0.025N tare load, and the normal equilibrium force can be recorded. The dynamic shear test can be performed at a shear strain of 0.01 radians on a logarithmic frequency sweep (0.01-20 Hz), and the complex shear modulus and phase shift angle can be calculated (Zhu et al, 1993). Since pulp mechanical properties have not been reported, gel mechanical properties can be compared to those of the as-isolated native pulp tissue tested under similar conditions.

### Example 22

### Characterization of Initial Cell Response in Hydrogel Scaffolds

The attachment and growth morphology (n=3) of pulp cells or MSCs as a function of culture time (1, 3, 7, 14, 21, 28 days) can be examined using histological staining and environmental scanning electron microscopy (ESEM). At the designated time points, the samples can be washed three times with PBS to remove non-adherent cells. For histochemical staining, the samples can be fixed in neutral formalin, dehydrated in ethanol, and embedded in paraffin. The samples can be sectioned and stained with hematoxylin and eosin. The samples can then be sectioned and stained with hematoxylin and eosin. For ESEM analysis, the samples can be fixed in 2% glutaraldehyde in 0.1M cacodylate buffer at 37° C. immediately after dissection. The samples can be imaged in both environmental and high vacuum modes of the scanning electron microscope (FEI Company). The advantage of the ESEM is the ability to image hydrated samples without artifacts which can arise from preparation methods required for high vacuum SEM analysis. Cell proliferation: Total DNA content can be quantified using the PicoGreen® dsDNA (Molecular Probe, Eugene, Oreg.) assay following the manufacturer's suggested protocol. The harvested samples can be washed with PBS and homogenized in DI water, and 25 μL of the sample can be added to 175 μL of the PicoGreen® reagent. Sample fluorescence can be measured with a microplate reader (Tecan, Research Triangle Park, N.C.) with excitation and emission wavelengths set at 485 nm and 535 nm respectively.

### Example 23

### Characterization of Pulp Cell Differentiation in Hydrogel Scaffolds

For cell differentiation, alkaline phosphatase (ALP) activity, glycosaminoglycan (GAG) content and total collagen production can be quantified using standard assays. ALP activity can be quantified using a calorimetric assay as described previously. The samples can be incubated at 37° C. for 30 minutes in 0.1 M Na2CO3 buffer containing 2 mM MgCl2 with disodium p-nitrophenyl phosphate (pNP—PO4) as the substrate. Standard solutions can be prepared by serial dilutions of 0.5 mM p-nitrophenol (pNP) in Na2CO3 buffer. Enzymatic activity can then be expressed as total nmoles of pNP produced per min per total cell number. Absorbance can be measured at 415 nm by a Spectrafluor reader (Tecan, Research Triangle Park, N.C.). Total collagen content can be quantified using calorimetric hydroxyproline quantification assay. Briefly, samples can be homogenized and digested in 2% Papain. 100 μL of the digests can be hydrolyzed for 30 minutes at 120° C. Subsequently 50 μL of the hydrolyzed solution can be added to 450 μL of 56 mM of chloramines T (Sigma, St Louis, Mo.) solution. After 25 minutes, 500 μL of Ehrlich's reagent is added and incubated at 65° C. for 20 minutes. Absorbance can be measured at 550 nm with a microplate reader (Tecan). Total GAG content can be quantified with Blyscan 1,9-dimethylmethylene blue (DMMB) assay (Accurate Chemical, Westbury, N.Y.) using the manufacturer's suggested protocol. Briefly, samples can be homogenized and digested in 2% Papain and 100 μL of the digest can be added to 1 ml of the Blyscan assay dye agent and mixed for an hour. The mixture can be centrifuged for 20 minutes at 10,000 g and the precipitate can be re-suspended in 1 mL of Blyscan dissociation solution. The absorbance can be measured at 620 nm using a microplate reader (Tecan). Matrix Organization and Distribution: the distribution of types I and III collagen, procollagen, ALP, DSP and GAG can be analyzed via immuno-histochemical techniques following the methods of Gronthos et all (Goldberg and Smith, 2004; Gronthos et al, 2000). Briefly, appropriate dilutions of the primary antibody (Chemicon, Temecula, Calif.) for the individual protein can be allowed to react with the substrate, after which a secondary antibody (Jackson Immuno Laboratories, West Grove, Pa.) conjugated with a fluorescent marker can be added to the system. 0.5% goat serum (Jackson Immuno Laboratories, West Grove, Pa.) can be used as a blocking solution to prevent non-specific binding of antibodies. The distribution of fluorescence can be viewed using a Zeiss Axiovert 200 fluorescence microscope (Carl Zeiss Inc., Thornwood, N.Y.). Fluorescence intensity, which is correlated to the amount of protein present, can be quantified using a fluorimetric assay, where protein concentration is calculated based on a standard curve (protein concentration vs. fluorescence intensity) generated using various concentrations of the protein to be detected. Sample fluorescence can be measured using a microplate reader (Tecan). Although mineralization is not expected of pulp cells in the hydrogel, the ectopic formation of mineralized nodules by these cultures in 2-D and 3-D cultures can be examined by using Alizarin Red S (ALZ) assay staining specific for calcium. The samples can be washed in ddH2O, and incubated in 40 mM Alizarin red solution for 10 min. After additional washes, the scaffolds can be incubated in 10% cetyl pyridinium chloride for 15 min to solubilize reacted ALZ. In this assay, serial dilutions of 1N CaCl2 can be used as standards. ALZ concentration per cell can be calculated as molar equivalent CaCl2 divided by the average cell number. Absorbance can be measured at 570 nm using a microplate reader (Tecan) The gene expression of osteocalcin and dentin sialophosphoprotein (DSP) can be detected by reverse transcription followed by polymerase chain reaction (RT-PCR). For this purpose, cells can be released from hydrogel by chitinase, and from collagen gels using collagenase digestion. RNA can then be isolated by phase separation and further purified using RNeasy kit (Qiagen, Valencia, Calif.) followed by DNase digestion. First strand cDNA can be synthesized using Superscript First-Strand Synthesis System (Invitrogen, Carlsbad, Calif.). RT-PCR can be performed using the icycler IQ™ Real-Time Detection system. Primers and probes for allgenes of interest can be designed using Beacon Designer Probe/Primer Design Software (Bio-Rad, Hercules, Calif.): osteocalcin, sense 5′-CATGAGAGCCCTCACA-3′ and antisense 5′ AGAGCGACACCCTAGAC-3′; dentin sialoprotein (DSP), sense 5′ GGCAGTGACTCAAAAGGAGC-3′ and antisense 5′-TCATATTTGGCAGGTTTTTCT 3′12,87, GAPDH, sense 5′ TCCACCCACGGCAAGTTCA-3′, antisense 5′-GAGATGATGACCCTCTTGGCG-3′, probe 5′-ACCCTTCAAGTGAGCCCCAGCCTTC 3′. Real-time PCR can be performed using the following conditions: after 3 min at 95° C., 45 PCR cycles can be performed with each cycle consisting of 15 sec at 95° C. followed by 30 seconds at 58° C. and 15 seconds at 70° C. The level of target mRNA expression can be calculated relative to reference GAPDH mRNA.

### Example 24

### Characterization of Angiogenic Response in Pulp Cell Cultures

In addition to the cell-related outcome measures described above, angiogenesis with the hydrogel can be determined in terms of the formation of tubular structures as well as positive staining for endothelial membrane protein CD-31 and von Willebrand's factor (vWF) in order to identify vessels lined with endothelial cells. The formation of tubular structures due to PRP stimulation can be quantified using digital image analysis, by measuring the perimeter, area, or number of branches observed in the network following published methods (Tran-Hung et al, 2006; Salani et al, 2000). In addition, the production of VEGF, PDGF and bFGF by pulp cells can be determined via immunohistochemistry, and the concentration of these angiogenic factors in the cell culturing media can also be measured by ELISA (d'Aquino et al, 2007; Tsay et al, 2005).

### Example 25

### Data and Statistical Analysis

For data analysis, primarily two-way analysis of variance (ANOVA) can be performed to determine any statistically significant relationships between factors examined in the proposed experiments. Significance can be tested at p<0.05. Aim 1. The specific cell-related outcome measures are 1) cell proliferation, 2) cell differentiation (ALP activity, GAG, total collagen and mineralization). The variables can be gel concentration, cell seeding density, gel type and culturing time. The scaffold-related outcomes include gel geometry, weight, and mechanical properties as a function of gel concentration, cell seeding density and time. Multi-way ANOVA can be used to correlate variable effects on cell growth and differentiation, as well as gel properties. Cell-related and scaffold-related outcomes 2-D vs. 3-D culture, gel type, mode of PRP delivery, PRP concentration and time can be analyzed as above. Multi-way ANOVA can be used to correlate variable-effects on cell growth and differentiation. Once a significant difference is predicted by ANOVA, the Tukey-Kramer significance test can be used to compare between the group means. Statistical significance can be tested at p<0.05. All statistical analysis can be performed using the JMP statistic software (SAS Institute).

### Example 26

### Antibacterial Potential of Chitosan

The antibacterial benefit of chitosan in pulp regeneration can be examined, and antibiotic delivery can be incorporated into the optimal hydrogel scaffold. The selection criteria for the optimal scaffold for pulp tissue engineering can be based on cell response and scaffold properties as determined by in vitro culture, and two different hydrogels can show similar results. Consequently, scaffold selection can be based on the priority of certain parameters, and the totality of cellular response (e.g., 3 or more characteristics). Moreover, depending on the responses observed, other hydrogel scaffolds can be assessed, as one scaffold can be more advantageous for angiogenesis while the other more effective for cell proliferation. The design of composite scaffolds for pulp tissue engineering can be examined to those ends.

### Example 27

### Increasing Angiogenic Responses

In addition to PRP, co-culture of pulp cells with endothelial cells and increasing the concentration of growth factor levels by adding exogenous VEGF, PDGF or FGF-2 can also be used to induce angiogenesis. Given a concentration of each of these factors in PRP, one can scale up the factor concentrations in these cultures.

### Example 28

### Mineralization Induced by PRP

Mineralization by pulp cells under PRP stimulation can be exploited for dentin formation. Mineralization can be monitored in pulp cultures in response to the addition of PRP. To prevent mineralization one skilled in the art can switch to exogenous factors for vascularization to prevent unwanted mineralization in the pulp. PRP contains a host of factors that can be ideal for simultaneous stimulation of multiple cell populations and for engineering complex tissues.

### Example 29

### Pulp Regeneration vs. Pulp-Dentin Regeneration

For clinical application of the tissue engineered pulp graft, it is possible to have several levels of regeneration and still improve clinical outcomes. The optimal level is the complete regeneration of pulpal structure and function, including revascularization, odontoblast function and complete innervation. Pulp-mediated dentin formation which can be a critical aspect in total tooth therapy. Since a healthy and functional pulp is a useful for dentin repair, enhanced pulp-mediated dentin can be used to demonstrate the functionality of the engineered pulp as well as provide potential solutions for the complete regeneration of tooth structure.

### OTHER EMBODIMENTS

It is to be understood that while the disclosed subject matter has been described in conjunction with the detailed description thereof, the foregoing description is intended to illustrate and not limit the scope of the disclosed subject matter, which is defined by the scope of the appended claims. Other aspects, advantages, and modifications are within the scope of the following claims.

### REFERENCES

About et al., Exp. Cell Res. 258:33-41, 2000
About et al., J. Biomed. Mater Res. (Appl. Biomater) 63:418-413, 2002
About, I., Bottero, M. J., de Denato, P., Camps, J., Franquin, J. C., and Mitsiadis, T. A.: Human dentin production in vitro. Exp. Cell Res. 258:33, 2000.
About, I., Mitsiadis, T. A., Bottero, M.-J., Butler, W., Franquin, J.-C., and Camps, J.: Influence of resinous monomers on the differentitation in vitro of human pulp cells into odontoblasts. J. Biomed. Mater. Res. (Appl. Biomater.) 63:418, 2002.
About, I.; Bottero, M. J.; de Denato, P.; Camps, J.; Franquin, J. C.; Mitsiadis, T. A. Human dentin production in vitro. Exp. Cell Res. 2000, 258, 33-41.
ADA. Survey of Dental Practice. 5S99. 1999. American Dental Association. Survey of Dental Services. (GENERIC)
Almany, L.; Seliktar, D. Biosynthetic hydrogel scaffolds made from fibrinogen and polyethylene glycol for 3D cell cultures. Biomaterials 2005, 26 (15), 2467-2477.
Almushayt, A., Saki, A., Ramachandran, A., Narayanay, K., Hao, J., and George, A. The in-vivo role of DMP2 in the cytodifferentiation of undifferentiated mesenchymal pulp cells into ododonblast-like cells. Presented at IADR 2002. (GENERIC)
Andreasen, J. O., Borum, M. K., and Andreasen, F. M.: Replantation of 400 avulsed permanent incisors. 3. Factors related to root growth. Endod. Dent. Traumatol. 11:69, 1995.
Andreasen, J. O., Borum, M. K., Jacobsen, H. L., and Andreasen, F. M.: Replantation of 400 avulsed permanent incisors. 1. Diagnosis of healing complications. Endod. Dent. Traumatol. 11:51, 1995.
Andreasen, J. O., Borum, M. K., Jacobsen, H. L., and Andreasen, F. M.: Replantation of 400 avulsed permanent incisors. 2. Factors related to pulp healing. Endod. Dent. Traumatol. 11:59, 1995.
Anitua, E.; Andia, I.; Sanchez, M.; Azofra, J.; del Mar, Z. M.; de la, F. M.; Nurden, P.; Nurden, A. T. Autologous preparations rich in growth factors promote proliferation and induce VEGF and HGF production by human tendon cells in culture. J. Orthop. Res. 2005, 23 (2), 281-286.
Banes, A. J., Tsuzaki, M., Yamamoto, J., Fischer, T., Brigman, B., Brown, T., and Miller, L.: Mechanoreception at the cellular level: the detection, interpretation, and diversity of responses to mechanical signals. Biochem. Cell Bio. 73:349, 1995.
Barkhordar R A, Ghani Q P, Russel T R, and Hussain M Z: Interleukin-1B Activity and Collagen Synthesis in Human Dental Pulp Fibroblasts. Journal of Endodontics 28:157, 2002.
Barthel et al., J. Endod. 26:525-528, 2000
Baumgartner, J. C. Pulpal infections including caries. In Seltzer and Bender's: Dental Pulp, Hargreaves, K. M., Goodis, H. E., Eds.; Quintessence Pulblishing Co, Inc: Chicago, 2002.
Baumgartner, J. C.: Pulp infections including caries. In Hargreaves, K. M. and Goodis, H. E., (eds.): Seltzer and Bender's: Dental Pulp. Chicago, Quintessence Publishing Co, Inc, 2002,
Bimstein, E. and Shoshan, S.: Enhanced healing of tooth-pulp wounds in the dog by enriched collagen solution as a capping agent. Arch. Oral Biol. 26:97, 1981.
Bohl et al., J. Biomater. Sci. Polym. Ed 9:749-764, 1998
Bohl et al., J. Biomaterial Sci. Polym. Ed. 9:749-764, 1998
Bohl, K. S., Shon, J., Rutherford, B., and Mooney, D. J.: Role of synthetic extracellular matrix in development of engineered dental pulp. J. Biomater. Sci. Polym. Ed 9:749, 1998.
Bouhadir et al., J. Drug Target 9:397-406, 2001
Bouvier et al., Arch. Oral Bio. 35:2031-309, 1990
Bouvier et al., Arch. Oral Biol. 35:301-309, 1990
Bouvier, M., Joffre, A., and Magloire, H.: In vitro mineralization of a three-dimensional collagen matrix by human dental pulp cells in the presence of chondroitin sulphate. Arch. Oral Biol. 35:301, 1990.
Briggs, P. F. A. and Scott, B. J.: Evidence-based dentistry: endodontic failure—how should it be managed? Br. Dent. J 184:159, 1997.
Brock et al, J. Dent. Res. 81:203-208, 2002
Brock, D. P., Marty-Roix, R., and Spector, M.: Alpha-smooth-muscle action in and contraction of porcine dental pulp cells. J. Dent. Res. 81:203, 2002.
Buurma, B., Gu, K., and Rutherford, R. B.: Transplantation of human pulp and gingival fibroblasts attached to synthetic scaffolds. Eur. J. Oral Sci. 107:282, 1999.
Cheung, G. S.: Endodontic failures-changing the approach. Inter. Dent. J. 46:131, 1996.
Cho, B. C.; Kim, J. Y.; Lee, J. H.; Chung, H. Y.; Park, J. W.; Roh, K. H.; Kim, G. U.; Kwon, I. C.; Jang, K. H.; Lee, D. S.; Park, N. W.; Kim, I. S. The bone regenerative effect of chitosan microsphereencapsulated growth hormone on bony consolidation in mandibular distraction osteogenesis in a dog model. Journal of Craniofacial. Surgery. 15. (2):299-311.; discussion. 312-3, 2004.
Choi, S. H.; Kim, C. K.; Cho, K. S.; Huh, J. S.; Sorensen, R. G.; Wozney, J. M.; Wikesjo, U. M. Effect of recombinant human bone morphogenetic protein-2/absorbable collagen sponge (rhBMP-2/ACS) on healing in 3-wall intrabony defects in dogs. J. Periodontol. 2002, 73 (1), 63-72.
Couble et al., Calcif. Tissue Int. 66:129-138, 2000
Couble, M. L.; Farges, J. C.; Bleicher, F.; Perrat-Mabillon, B.; Boudeulle, M.; Magloire, H. Odontoblast differentiation of human dental pulp cells in explant cultures. Calcif. Tissue Int. 2000, 66, 129-138.
Couble, M. L., Farges, J. C., Bleicher, F., Perrat-Mabillon, B., Boudeulle, M., and Magloire, H.: Odontoblast differentiation of human dental pulp cells in explant cultures. Calcif. Tissue Int. 66:129, 2000.
Cox, C. F.; Subay, R. K.; Ostro, E.; Suzuki, S.; Suzuki, S. H. Tunnel defects in dentin bridges: their formation following direct pulp capping. Oper. Dent. 1996, 21 (1), 4-11.
Cvek, M., Cleaton-Jones, P., Austin, J., Lownie, J., Kling, M., and Fatti, P.: Effect of topical application of doxycycline on pulp revascularization and periodontal healing in reimplanted monkey incisors. Endod. Dent. Traumatol. 6:170, 1990.
Cvek, M., Cleaton-Jones, P., Austin, J., Lownie, J., Kling, M., and Fatti, P.: Pulp revascularization in reimplanted immature monkey incisors-predictability and the effect of antibiotic systemic prophylaxis. Endod. Dent. Traumatol. 6:157, 1990.
Cvek, M.: A clinical report on partial pulpotomy and capping with calcium hydroxide in permanent incisors with complicated crown fracture. J. Endod. 4:232, 1978.
Dahlen, G. and Haapasalo, M.: Microbiology of apical periodontisis. In Orstavik, D. and Pitt Ford, T. R., (eds.): Essential Endodontology: Prevention and Treatment of Apical Periodontitis. Oxford, Blackwell Science, 1998,
Dahlen, G.; Haapasalo, M. Microbiology of apical periodontisis. In Essential Endodontology: Prevention and Treatment of Apical Periodontitis, Orstavik, D., Pitt Ford, T. R., Eds.; Blackwell Science: Oxford, 1998.
d'Aquino, R.; Graziano, A.; Sampaolesi, M.; Laino, G.; Pirozzi, G.; De, R. A.; Papaccio, G. Human postnatal dental pulp cells co-differentiate into osteoblasts and endotheliocytes: a pivotal synergy leading to adult bone tissue formation. Cell Death. Differ. 2007, 14 (6), 1162-1171.
Das, S., Das, A. K., and Murphy, R. A.: Experimental apexigenesis in baboons. Endod. Dent. Traumatol. 13:31, 1997.
Derringer, K. A.; Linden, R. W. Angiogenic growth factors released in human dental pulp following orthodontic force. Arch. Oral Biol. 2003, 48 (4), 285-291.
Derringer, K. A.; Linden, R. W. Enhanced angiogenesis induced by diffusible angiogenic growth factors released from human dental pulp explants of orthodontically moved teeth. Eur. J. Orthod. 1998, 20 (4), 357-367.
Derringer, K. A.; Linden, R. W. Vascular endothelial growth factor, fibroblast growth factor 2, platelet derived growth factor and transforming growth factor beta released in human dental pulp following orthodontic force. Arch. Oral Biol. 2004, 49 (8), 631-641.
Dikovsky, D.; Bianco-Peled, H.; Seliktar, D. The effect of structural alterations of PEG-fibrinogen hydrogel scaffolds on 3-D cellular morphology and cellular migration. Biomaterials 2006, 27 (8), 1496-1506.
Elbert, D. L.; Hubbell, J. A. Conjugate addition reactions combined with free-radical cross-linking for the design of materials for tissue engineering. Biomacromolecules. 2001, 2 (2), 430-441.
Elisseeff, J.; McIntosh, W.; Anseth, K.; Riley, S.; Ragan, P.; Langer, R. Photoencapsulation of chondrocytes in poly(ethylene oxide)-based semi-interpenetrating networks. J. Biomed. Mater. Res. 2000, 51 (2), 164-171.
Etienne, O.; Schneider, A.; Taddei, C.; Richert, L.; Schaaf, P.; Voegel, J. C.; Egles, C.; Picart, C. Degradability of polysaccharides multilayer films in the oral environment: an in vitro and in vivo study. Biomacromolecules. 6. (2):726.-33., 2005.
Friedman, S.: Treatment outcome and prognosis of endodontic therapy. In Orstavik, D. and Pitt Ford, T. R., (eds.): Essential Endodontology: Prevention and Treatment of Apical Periodontitis. Oxford, Blackwell Science, 1998,
Fuks, A. B., Jones, P. C., Michaeli, Y., and Bimstein, E.: Pulp response to collagen and glutaraldehyde in pulpotomized primary teeth of baboons. Pediatr. Dent. 13:142, 1991.
Fuks, A. B., Michaeli, Y., Sofer-Saks, B., and Shoshan, S.: Enriched collagen solution as a pulp dressing in pulpotomized teeth in monkeys. Pediatr. Dent. 6:243, 1984.
Goldberg, M.; Smith, A. J. Cells and extracellular matrices of dentin and pulp: A biological basis for repair and tissue engineering. Critical Reviews in Oral Biology & Medicine 2004, 15 (1), 13-27.
Grando, M. L.; Westphalen, B. L.; de Figueiredo, J. A.; Nor, J. E.; de Araujo, F. B.; Fossati, A. C. Vascular endothelial growth factor and its relationship with the dental pulp. J. Endod. 2007, 33 (5), 524-530.
Gronthos et al., J. Dent. Res., 2002, 81:531-535
Gronthos et al., Proc. Nat. Acad. Sci., 2000, 97:13625-13630
Gronthos, S.; Brahim, J.; Li, W.; Fisher, L. W.; Cherman, N.; Boyde, A.; DenBesten, P.; Robey, P. G.; Shi, S. Stem cell properties of human dental pulp stem cells. J. Dent. Res. 2002, 81, 531-535.
Gronthos, S.; Mankani, M.; Brahim, J.; Robey, P. G.; Shi, S. Postnatal human dental pulp stem cells (DPSCs) in vitro and in vivo. PNAS 2000, 97, 13625-13630.
Guven, G.; Altun, C.; Gunhan, O.; Gurbuz, T.; Basak, F.; Akbulut, E.; Cehreli, Z. C. Co-expression of cyclooxygenase-2 and vascular endothelial growth factor in inflamed human pulp: an immunohistochemical study. J. Endod. 2007, 33 (1), 18-20.
Hao, J., Shi, S., Niu, Z., Xun, Z., Yue, L., and Xiao, M.: Mineralized nodule formation by human dental papilla cells in culture. Eur. J. Oral Sci. 105:318, 1997.
Hargreaves, K. M. and Goodis, H. E.: Seltzer and Bender's: Dental Pulp. Chicago, Quintessence Publishing Co, Inc, 2002,
Hasselgren G, Larsson Å, Rundquist L (1977) Pulpal status following autogenous transplantation of fully developed maxillary canines. Oral Surg. 44:106-112
Herrick, S.; Blanc-Brude, O.; Gray, A.; Laurent, G. Fibrinogen. Int. J. Biochem. Cell Biol. 1999, 31 (7), 741-746.
Holmes et al, 2002). (Biomech. Model. Mechanobiol. 2002, 1:59-67
Jepsen, S., Albers, H. K., Fleiner, B., Tucker, M., and Rueger, D.: Recombinant human osteogenic protein-1 induces dentin formation: an experimental study in miniature swine. J. Endod. 23:378, 1997.
Kaigler, D. and Mooney, D.: Tissue engineering's impact on dentistry. J. Dent. Educ. 65:456, 2001.
Katti et al., J. Biomed. Mater. Res. B. Appl. Biomater., 2004, 70:286-296).
Kishi, Y.; Takahashi, K. Change of Vascular Architecture of Dental Pulp with Growth. In Dynamic Aspects of Dental Pulp, Inoki, R., Kudo, T., Olgart, L., Eds.; Chapman and Hall: London, 1995; pp 97-129.
Kling, M., Cvek, M., and Mejare, I.: Rate and predictability of pulp revascularization in therapeutically reimplanted permanent incisors. Endod. Dent. Traumatol. 2:83, 1986.
Klokkevold, P. R.; Fukayama, H.; Sung, E. C.; Bertolami, C. N. The effect of chitosan (poly-N-acetyl glucosamine) on lingual hemostasis in heparinized rabbits. J. Oral Maxillofac. Surg. 1999, 57 (1), 49-52.
Koran et al., J. Amer. Dent. Assoc. 95:75-79, 1977
Landesberg, R.; Roy, M.; Glickman, R. S. Quantification of growth factor levels using a simplified method of platelet-rich plasma gel preparation. J. Oral Maxillofac. Surg. 2000, 58 (3), 297-300.
Lu, H. H.; El Amin, S. F.; Scott, K. D.; Laurencin, C. T. Three-dimensional, bioactive, biodegradable, polymer-bioactive glass composite scaffolds with improved mechanical properties support collagen synthesis and mineralization of human osteoblast-like cells in vitro. J. Biomed. Mater. Res 2003, 64A (3), 465-474.
Lu, H. H.; Jiang, J.; Tang, A.; Hung, C. T.; Guo, X. E. Development of Controlled Heterogeneity on a Polymer-Ceramic Hydrogel Scaffold for Osteochondral Repair. Bioceramics 2005, 17 (Key Engineering Materials Vols. 284-286), 607-610.
Lu, H. H.; Kofron, M. D.; El Amin, S. F.; Attawia, M. A.; Laurencin, C. T. In vitro bone formation using muscle-derived cells: a new paradigm for bone tissue engineering using polymer-bone morphogenetic protein matrices. Biochemical. & Biophysical Research. Communications. 2003b, 305 (4), 882-889.
Lutolf, M. P.; Hubbell, J. A. Synthesis and physicochemical characterization of end-linked poly(ethylene glycol)-co-peptide hydrogels formed by Michael-type addition. Biomacromolecules. 2003, 4 (3), 713-722.
Ma, L.; Gao, C.; Mao, Z.; Zhou, J.; Shen, J.; Hu, X.; Han, C. Collagen/chitosan porous scaffolds with improved biostability for skin tissue engineering. Biomaterials 2003, 24 (26), 4833-4841.
Mathieu, S.; El-Battari, A.; Dejou, J.; About, I. Role of injured endothelial cells in the recruitment of human pulp cells. Arch. Oral Biol. 2005, 50 (2), 109-113.
Mauck, R L., Soltz, M. A., Wang, C. C., Wong, D. D., Chao, P. H., Valhmu, W. B., Hung, C. T., and Ateshian, G. A.: Functional tissue engineering of articular cartilage through dynamic loading of chondrocyte-seeded agarose gels. J. Biomech. Eng. 122:252, 2000.
Mejare, I. and Cvek, M.: Partial pulpotomy in young permanent teeth with deep carious lesions. Endod. Dent. Traumatol. 9:238, 1993.
Mesaros, S. V. and Trope, M.: Revascularization of traumatized teeth assessed by laser Doppler flowmetry: case report. Endod. Dent. Traumatol. 13:24, 1997.
Mjör, I. A.; Heyeraas, K. J. Pulp-Dentin and Periodontal Anatomy and Physiology. In Essential Endodontology, Orstavik, D., Pitt Ford, T. R., Eds.; Blackwell Science: New York, 1998; pp 9-41.
Mooney, D. J., Powell, C., Piana, J., and Rutherford, B.: Engineering dental pulp-like tissue in vitro. Biotechnol. Prog. 12:865, 1996.
Mori, T.; Okumura, M.; Matsuura, M.; Ueno, K.; Tokura, S.; Okamoto, Y.; Minami, S.; Fujinaga, T. Effects of chitin and its derivatives on the proliferation and cytokine production of fibroblasts in vitro. Biomaterials 1997, 18 (13), 947-951.
Murray, M. M.; Spindler, K. P.; Ballard, P.; Welch, T. P.; Zurakowski, D.; Nanney, L. B. Enhanced histologic repair in a central wound in the anterior cruciate ligament with a collagen-platelet-rich plasma scaffold. J Orthop Res. 2007 August; 25(8): 1007-17.
Murray, M. M.; Spindler, K. P.; Devin, C.; Snyder, B. S.; Muller, J.; Takahashi, M.; Ballard, P.; Nanney, L. B.; Zurakowski, D. Use of a collagen-platelet rich plasma scaffold to stimulate healing of a central defect in the canine ACL. J. Orthop. Res. 2006, 24 (4), 820-830.
Muzzarelli, R. A.; Biagini, G.; Bellardini, M.; Simonelli, L.; Castaldini, C.; Fratto, G. Osteoconduction exerted by methylpyrrolidinone chitosan used in dental surgery. Biomaterials 1993, 14 (1), 39-43.
Muzzarelli, R.; Biagini, G.; Pugnaloni, A.; Filippini, O.; Baldassarre, V.; Castaldini, C.; Rizzoli, C. Reconstruction of parodontal tissue with chitosan. Biomaterials 1989, 10 (9), 598-603.
Myers, W. C. and Fountain, S. B.: Dental pulp regeneration aided by blood and blood substitutes after experimentally induced periapical infection. Oral Surg. 37:441, 1974.
Nakashima, M. Establishment of primary cultures of pulp cells from bovine permanent incisors. Arch. Oral Biol. 1991, 36, 655-663.
Nakashima, M., Mizunuma, K., Murakami, T., and Akamine, A.: Induction of dental pulp stem cell differentiation into odontoblasts by electroporation-mediated gene delivery of growth/differentiation factor 11 (Gdfl 1). Gene Ther. 9:814, 2002.
Nakashima, M.: Induction of dentine in amputated pulp of dogs by recombinant human bone morphogenetic proteins-2 and -4 with collagen matrix. Arch. Oral Biol. 39:1085, 1994.
Nevins, A., Finkelstein, F., Laporta, R., and Borden, B. G.: Induction of hard tissue into pulpless open-apex teeth using collagen-calcium phosphate gel. J. Endod. 4:76, 1978.
Nevins, A., Finkelstein, F., Laporta, R., Borden, B. G., and Moodnik, R.: Formation of mineralized scar tissue induced by implants containing collagen-calcium phosphate gel. J. Endod. 1:303, 1975.
Nevins, A. J., Finkelstein, F., Borden, B. G., and Laporta, R.: Revitalization of pulpless open apex teeth in rhesus monkeys, using collagen-calcium phosphate gel. J. Endod. 2:159, 1976.
Nevins, A. J., LaPorta, R. F., Borden, B. G., and Spangberg, L. S.: Pulpotomy and partial pulpectomy procedures in monkey teeth using cross-linked collagen-calcium phosphate gel. Oral Surg. Oral Med. Oral Pathol. 49:360, 1980.
Nguyen, K. T.; West, J. L. Photopolymerizable hydrogels for tissue engineering applications. Biomaterials 2002, 23 (22), 4307-4314.
Nishimura, K.; Nishimura, S.; Seo, H.; Nishi, N.; Tokura, S.; Azuma, I. Effect of multiporous microspheres derived from chitin and partially deacetylated chitin on the activation of mouse peritoneal macrophages. Vaccine 1987, 5 (2), 136-140.
Nyborg, H. Healing processes in the pulp on capping; a morphologic study; experiments on surgical lesions of the pulp in dog and man. Acta Odontol. Scand. 1955, 13 (suppl. 16), 1-130.
Okiji, T.: Pulp as a connective tissue. In Hargreaves, K. M. and Goodis, H. E., (eds.): Seltzer and Bender's: Dental Pulp. Chicago, Quintessence Publishing Co, Inc, 2002,
Park, D. J.; Choi, B. H.; Zhu, S. J.; Huh, J. Y.; Kim, B. Y.; Lee, S. H. Injectable bone using chitosanalginate gel/mesenchymal stem cells/BMP-2 composites. Journal of Cranio. Maxillo. Facial. Surgery. 33. (1):50-4, 2005.
Park, J. S.; Choi, S. H.; Moon, I. S.; Cho, K. S.; Chai, J. K.; Kim, C. K. Eight-week histological analysis on the effect of chitosan on surgically created one-wall intrabony defects in beagle dogs. J. Clin. Periodontol. 2003, 30 (5), 443-453.
Peters et al., J. Biochem. Mater. Res. 60:668-678, 2002
Rabea, E. I.; Badawy, M. E.; Stevens, C. V.; Smagghe, G.; Steurbaut, W. Chitosan as antimicrobial agent: applications and mode of action. Biomacromolecules. 2003, 4 (6), 1457-1465.
Ranly, D. M. and Garcia-Godoy, F.: Current and potential pulp therapies for primary and young permanent teeth. J. Dent. 28:153, 2000.
Reisbick, J. Dent. Res. 52:407-411, 1973
Richardson et al. Nat. Biotechnol. 2001, 19:1029-1034
Roeder, B. A., Kokini, K., Sturgis, J. E., Robinson, J. P., and Voytik-Harbin, S. L.: Tensile mechanical properties of three-dimensional type I collagen extracellular matrices with varied microstructure. J. Biomed. Eng. 124:214, 2002.
Rungvechvuttivittaya, S., Okiji, T., and Suda, H.: Responses of macrophage-associated antigen-expressing cells in the dental pulp of rat molars to experimental tooth replantation. Arch. Oral Biol. 43:701, 1998.
Rutherford, B. and Fitzgerald, M.: A new biological approach to vital pulp therapy. Crit. Rev. Oral Biol. Med. 6:218, 1995.
Rutherford, R. B. and Gu, K.: Treatment of inflamed ferret dental pulps with recombinant bone morphogenetic protein-7. Eur. J. Oral Sci. 108:202, 2000.
Rutherford, R. B.: BMP-7 gene transfer to inflamed ferret dental pulps. Eur. J. Oral Sci. 109:422, 2001.
Salani, D.; Taraboletti, G.; Rosano, L.; Di, C., V; Borsotti, P.; Giavazzi, R.; Bagnato, A. Endothelin-1 induces an angiogenic phenotype in cultured endothelial cells and stimulates neovascularization in vivo. Am. J. Pathol. 2000, 157 (5), 1703-1711.
Sapelli, P.; Baldassare, V.; Muzzarelli, R.; Emanuelli, M. Chitosan in Dentistry. In Chitin in Nature and Technology, 1986; pp 507-512.
Sarasam, A.; Madihally, S. V. Characterization of chitosan-polycaprolactone blends for tissue engineering applications. Biomaterials 2005, 26 (27), 5500-5508.
Seliktar, D. Extracellular stimulation in tissue engineering. Ann. N.Y. Acad. Sci. 2005, 1047, 386-394.
Six, N., Decup, F., Lasfargues, J. J., Salih, E., and Goldberg, M.: Osteogenic proteins (bone sialoprotien and bone morphogenetic protein-7) and dental pulp mineralization. Journal of Materials Science Materials in Medicine 13:225, 2002.
Six, N., Lasfargues, J. J., and Goldberg, M.: Differential repair responses in the coronal and radicular areas of the exposed rat molar pulp induced by recombinant human bone morphogenetic protein 7 (osteogenic protein 1). Arch. Oral Biol. 47:177, 2002.
Sjögren, U., Hagglund, B., Sundqvist, G., and Wing, K.: Factors affecting the long-term results of endodontic treatment. J. Endod. 16:498, 1990.
Skoglund and Hasselgren, Oral Surg. Oral Med. Oral Pathol. (1992) 74:789-95
Skoglund, A. and Tronstad, L.: Pulp changes in replanted and autotransplanted immature teeth of dogs. J. Endod. 7:309, 1981.
Skoglund, A., Tronstad, L., and Wallenius, K.: A microangiographic study of vascular changes in replanted and autotransplanted teeth of young dogs. Inter. J. Oral Surg. 12:31, 1983.
Skoglund, A.: Pulp changes in replanted and autotransplanted apicoectomized mature teeth of dogs. Int. J. Oral Surg. 10:111, 1981.
Skoglund, A.: Vascular changes in replanted and autotransplanted apicoectomized mature teeth of dogs. Inter. J. Oral Surg. 10:100, 1981.
Smith, C. S., Setchell, D. J., and Harty, F. J.: Factors influencing the success of conventional root canal therapy—a five-year retrospective study. Int. Endod. J. 26:321, 1993.
Suda, H.; Ikeda, H. The Circulation of the Pulp. In Seltzer and Bender's Dental Pulp,
Suh, J. K.; Matthew, H. W. Application of chitosan-based polysaccharide biomaterials in cartilage tissue engineering: a review. Biomaterials 2000, 21 (24), 2589-2598.
Temenoff, J. S.; Athanasiou, K. A.; LeBaron, R. G.; Mikos, A. G. Effect of poly(ethylene glycol) molecular weight on tensile and swelling properties of oligo(poly(ethylene glycol) fumarate) hydrogels for cartilage tissue engineering. J. Biomed. Mater. Res. 2002, 59 (3), 429-437.
Teng J; Hasselgren G; Lu H. H. Collagen hydrogel scaffold for pulp tissue engineering. Proceedings of the Society for Biomaterials. 2005.
Thresher, J. Biomech. 6:443-449, 1973
Toparli, M., Gokay, N., and Aksoy, T.: Analysis of a restored maxillary second premolar tooth by using three-dimensional finite element method. J. Oral Rehab. 26:157, 1999.
Tran-Hung, L.; Mathieu, S.; About, I. Role of human pulp fibroblasts in angiogenesis. J. Dent. Res. 2006, 85 (9), 819-823.
Tsay, R. C.; Vo, J.; Burke, A.; Eisig, S. B.; Lu, H. H.; Landesberg, R. Differential growth factor retention by platelet-rich plasma composites. J. Oral Maxillofac. Surg. 2005, 63 (4), 521-528.
Tziafas, D., Smith, A. J., and Lesot, H.: Designing new treatment strategies in vital pulp therapy. J. Dent. 28:77, 2000.
van Amerongen, J. P., Lemmens, I. G., and Tonino, G. J.: The concentration, extractability and characterization of collagen in human dental pulp. Arch. Oral Biol. 28:339, 1983.
Vermilyea et al., J. Dent. Res. 56:762-767, 1977
Vongsavan, N.; Matthews, B. The vascularity of dental pulp in cats. J. Dent. Res. 1992, 71 (12), 1913-1915.
Weiger, R., Axmann-Kremar, D., and Lost, C.: Prognosis of conventional root canal treatment reconsidered. Endod. Dent. Traumatol. 14:1, 1998.
Yanpiset, K. and Trope, M.: Pulp revascularization of replanted immature dog teeth after different treatment methods. Endod. Dent. Traumatol. 16:211, 2000.
Yanpiset, K., Vongsavan, N., Sigurdsson, A., and Trope, M.: Efficacy of laser Doppler flowmetry for the diagnosis of revascularization of reimplanted immature dog teeth. Dent. Traumatology 17:63, 2001.
Young, C. S., Terada, S., Vacanti, J. P., Honda, M., Bartlett, J. D., and Yelick, P. C.: Tissue engineering of complex tooth structures on biodegradable polymer scaffolds. J. Dent. Res. 81:695, 2002.
Zhang, J. Q.; Nagata, K.; Iijima, T. Scanning electron microscopy and immunohistochemical observations of the vascular nerve plexuses in the dental pulp of rat incisor. Anat. Rec. 1998, 251 (2), 214-220.
Zhu, W.; Mow, V. C.; Koob, T. J.; Eyre, D. R. Viscoelastic shear properties of articular cartilage and the effects of glycosidase treatments. J. Orthop. Res. 1993, 11 (6), 771-781.
Zielinski, B. A.; Aebischer, P. Chitosan as a matrix for mammalian cell encapsulation. Biomaterials 1994, 15 (13), 1049-1056.
Zilberman, U., Mass, E., and Sarnat, H.: Partial pulpotomy in carious permanent molars. American Journal of Dentistry 2:147, 1989.

What is claimed is:

1. A composition comprising:
(a) a matrix comprising: (i) type I collagen, (ii) type III collagen, (iii) alginate, (iv) chitosan, (v) PEG-fibrinogen, or any combination thereof, and
(b) pulpal cells.

2. The composition of claim 1, wherein the matrix comprises type I collagen at a concentration of about 0.3% to about 3.0%, about 0.3% to about 0.5%, or about 0.5% to about 3.0%.

3. The composition of claim 1, wherein the matrix comprises type I collagen at a concentration of 1 mg/ml to about 4 mg/ml, about 2 mg/ml to about 3 mg/ml, about 1 mg/ml to about 2 mg/ml or about 3 mg/ml to about 4 mg/ml.

4. The composition of claim 1, wherein the matrix comprises type I collagen and type III collagen, wherein the ratio of type I collagen to type III collagen comprises about 30%:70%, about 55%:45%, about 45%:55%, or about 70%:30%.

5. The composition of claim 1, wherein the matrix comprises alginate at a concentration of about 1.0% to about 5.0%, about 1.0% to about 3.0%, or about 3.0% to about 5.0%.

6. The composition of claim 5, wherein the matrix comprises CaCl₂, at a concentration of about 50 mM to about 100 mM, about 100 to about 200 mM or about 50 mM to about 200 mM.

7. The composition of claim 1, wherein the matrix comprises chitosan at a concentration of about 1% w/v to about 5% w/v, about 1% w/v to about 2.5% v/w or about 2.5% w/v to about 5% w/v.

8. The composition of claim 1, wherein the matrix comprises PEG-fibrinogen at a concentration of about 10% w/v.

9. The composition of claim 8, wherein the matrix comprises PEG-diacrylate at a concentration of about 1% v/w to about 4% w/v, about 1% w/v to about 2.5% w/v or about 2.5% w/v to about 4% w/v.

10. The composition of claim 1, wherein the viscosity of the matrix is less than 100,000 centipoise at 37° C.

11. The composition of claim 1, wherein the gelation pH of the matrix is about 6.0, 7.5, or 9.0.

12. The composition of claim 1, wherein the composition further comprises at least two antibiotics and at least one antibiotic is selected from the group consisting of ciprofloxacin, minicyclin, and metronidazole.

13. The composition of claim 1, wherein the matrix further comprises platelet-rich plasma.

14. The composition of claim 1, wherein the pulpal cells are seeded in the matrix at a density of about 0.5×10⁶ cells/ml, 1×10⁶ cells/ml, about 2×10⁶ cells/ml, about 3×10⁶ cells/ml or about 0.5×10⁶ cells/ml to about 3×10⁶ cells/ml.

15. The composition of claim 1, wherein the pulpal cells comprise: pulp-derived stem cells, progenitor cells, mesenchymal stem cells, pulp cells, bone marrow cells, embryonic stem cells, umbilical cord-derived, hematopoetic stem cells, endothelial cells, cells obtained from a cell culture or a combination thereof.

16. The composition of claim 15, wherein the composition comprises a ratio of stem cells to pulp cells.

17. The composition of claim 15, wherein the composition comprises a ratio of pulp cells to endothelial cells.

18. The composition of claim 15, wherein the composition comprises a ratio of stem cells to endothelial cells.

19. The composition of claim 15, wherein the composition comprises a ratio of hematopoetic cells to mesenchymal cells.

20. The composition of any one of claim 16-19, wherein the ratio is about 0.5 to about 1 or about 1 to about 1 or about 2 to about 1 or about 3 to about 1 or about 4 or about 1 to about 0.5 or about 1 to about 2 or about 1 to about 3 or about 1 to about 4.

21. The composition of claim 1, wherein the composition comprises at least one agent that is an antibiotic, an antifungal agent, a growth factor or an angiogenic factor.

22. The composition of claim 1, wherein the composition comprises at least one time released agent that is an antibiotic, an antifungal agent, a growth factor or an angiogenic factor.

23. A composition comprising:
(a) a scaffold of (i) an electrospun collagen mesh, (ii) an electrospun chitosan mesh, (iii) an electrospun alginate mesh, (iv) an extruded collagen mesh, (v) and extruded chitosan mesh, (vi) an extruded alginate mesh, (vii) a poly(lactide-co-glycolide) mesh, (viii) gelatin, or (iv) any combination thereof, and
(b) at least one antibiotic, anti-fungal agent, growth factor or angiogenic factor.

24. A composition comprising:
(a) a scaffold of (i) a nanofiber, (ii) an extracellular matrix, (iii) a degradable polymer, (iv) a mesh of crosslinked fibers, (v) an artificial or synthetic scaffold material, (vi) a polycaprolactone polymer, (vii) a poly-α-hydroxyester polymer, (viii) a polyanhydride polymer, (ix) a polygalactin, (x) a polycyanoacrylate, (xi) a polyphosphazene, (xii) a mesh fabric, or (xiii) any combination thereof, and
(b) at least one antibiotic, antifungal agent, growth factor or angiogenic factor.

25. The composition of claim 23 or 24, wherein the mesh is a reservoir for growth factors or angiogenic factors.

26. The composition of claim 23 or 24, wherein the mesh can degrade to permit vascular ingrowth, cell proliferation, cell infiltration or deposition of extracellular matrix by cells.

27. The composition of claim 23 or 24, wherein the mesh has an alignment.

28. The composition of claim 27, wherein the alignment of the mesh is aligned, partially aligned or unaligned.

29. The composition of claim 27, wherein the alignment of the mesh can guide the formation of blood vessels.

30. A method for treating a subject having a pulp disorder or pulp damage within the pulp chamber of a tooth, the method comprising administering the composition of claim 1 to the subject.

31. A method for treating a subject in need of a pulpectomy, a pulpotomy, a partial pulpotomy, pulp capping or root canal therapy, the method comprising administering the composition of claim 1, 23 or 24 to the subject.

32. The method of claim 30 or 31, wherein the composition is administered to a pulp chamber in a tooth.

33. The method of claim 32, wherein the pulp chamber is substantially free of native pulp cells.

34. The method of claim 30 or 31, wherein the composition is administered by insertion apically or coronally to the native pulp cells.

35. A kit comprising the composition of claim 1, 23 or 24 and instructions for use, wherein the kit comprises: (a) a medium suitable for maintenance of harvested human pulp cells, (b) a sealant suitable for sealing a tooth, (c) a chamber for culturing pulp cells on a matrix or any combination thereof, wherein the matrix comprises: (i) type I collagen, (ii) type III collagen, (iii) alginate, (iv) chitosan, (v) PEG-fibrinogen, or any combination thereof, and pulpal cells.

36. The composition of claim 1, 23 or 24, wherein the composition comprises a hydrogel, a mesh, a microsphere, or any combination thereof.

37. The composition of claim 1, 23 or 24, wherein the composition comprises a biodegradable polymer microsphere, a hydrogel microsphere, or any combination thereof.

38. The composition of claim 1, 23 or 24, wherein the composition comprises a nanofiber, an artificial scaffold material, or a synthetic scaffold material or any combination thereof.

39. The composition of claim 23, 24 or 38, wherein cells can grow on the scaffold.

40. A method for determining whether a matrix can affect the growth and differentiation of pulpal cells, wherein the method comprises:
(a) growing pulpal cells in the biodegradable gel matrix,
(b) measuring a biological indicator, and
(c) determining whether there is an increase or decrease in the biological indicator compared to a same biological indicator measured in pulpal cells not grown in a biological gel matrix, wherein an increase or a decrease in the biological indicator indicates that the matrix affects the growth or differentiation of pulpal cells.

41. The method of claim 40, wherein the biological response comprises: proliferation, differentiation, three-dimensional cell structure formation, vascularization or angiogenesis.

42. The method of claim 40, wherein the biodegradable gel matrix comprises: type I collagen, type III collagen, chitosan, PEG-fibrinogen or alginate), as well as the effect of cell culturing on gel matrix properties (gel contraction and matrix organization.

43. A method for determining whether culturing of pulpal cells on a biodegradable gel matrix affects properties of the matrix, wherein the method comprises:
(a) growing pulpal cells in the biodegradable gel matrix,
(b) measuring a parameter of the biodegradable gel matrix, and
(c) determining whether there is an increase or decrease in the parameter compared to the same parameter measured in a biodegradable matrix in which pulpal cells are not grown, wherein an increase or a decrease in the parameter indicates that culturing of pulpal cells on a biodegradable gel matrix affects properties of the matrix.

44. The method of claim 43, wherein the parameter is comprises gel contraction or matrix organization.
